(19) United States
(12) Patent Application Publication
(10) Pub. No.: US 2024/0148864 A1
(43) Pub. Date: May 9, 2024

(54) POLYSACCHARIDE ADJUVANTS FOR VIRUS VACCINES

(71) Applicant: Children's Medical Center Corporation, Boston, MA (US)

(72) Inventors: Ivan Zanoni, Brookline, MA (US); Francesco Borriello, Arlington, MA (US); Ofer Levy, Cambridge, MA (US); David J. Dowling, Milton, MA (US)

(73) Assignee: Children's Medical Center Corporation, Boston, MA (US)

(21) Appl. No.: 18/281,540

(22) PCT Filed: Mar. 11, 2022
(86) PCT No.: PCT/US2022/019923

§ 371 Date: Sep. 11, 2023

Related U.S. Application Data
(60) Provisional application No. 63/257,076, filed on Oct. 18, 2021.
(60) Provisional application No. 63/160,667, filed on Mar. 12, 2021.

(51) Int. Cl. A61K 39/39; A61K 39/145; A61K 39/215; A61P 31/14; A61P 31/16; A61P 37/04

(52) U.S. Cl. CPC A61K 39/39; A61K 39/145; A61K 39/215; A61P 31/14; A61P 31/16; A61P 37/04; A61K 2039/55583

(57) ABSTRACT

Provided herein are adjuvantation systems comprising fungal polysaccharides for use in Beta coronavirus (e.g., MERS-CoV, SARS-CoV-1, or SARS-CoV-2) and influenza (influenza A virus and influenza B virus) vaccines and immunogenic compositions comprising the adjuvantation system and a Beta coronavirus or influenza virus antigen.

### RELATED APPLICATIONS

This application claims the benefit under 35 U.S.C. § 119(e) of U.S. Provisional Application No. 63/160,667 entitled “POLYSACCHARIDE ADJUVANTS FOR SEVERE ACUTE RESPIRATORY SYNDROME-RELATED CORONAVIRUS (SARS-COV) VACCINES,” filed on Mar. 12, 2021, and U.S. Provisional Application No. 63/257,076 entitled “POLYSACCHARIDE ADJUVANTS FOR VIRUS VACCINES,” filed on Oct. 18, 2021, the entire contents of each of which are incorporated herein by reference.

### FEDERALLY SPONSORED RESEARCH

This invention was made with government support under Grant Nos: R01AI121066 and 75N93019C00044 awarded by the National Institutes of Health (NIH). The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Mar. 11, 2022, is named C123370205WO00-SEQ-ZJG and is 76,363 bytes in size.

### BACKGROUND

Epidemic and pandemic infectious diseases are frequently caused by viruses that are readily transmissible to humans from birds and other mammals. Such viruses include those of several families, including Flaviviruses, Coronaviruses, Orthomyxoviruses, Paramyxoviruses, Rhabdoviruses, and Filoviruses. Among those of the greatest concern are Beta coronaviruses and influenza viruses, both of which are highly transmissible to and between humans and are often virulent.

The pandemic potential for these viruses is evident. Severe acute respiratory syndrome coronavirus (SARS-CoV or SARS-CoV-1) and severe acute respiratory syndrome coronavirus 2 (SARS-CoV-2) are the Beta coronaviruses responsible for causing the 2002-2004 outbreak of severe acute respiratory syndrome (SARS) and the ongoing 2019-2021 pandemic of coronavirus disease 2019 (COVID-19), respectively. Influenza A and influenza B viruses are responsible for causing both seasonal flu epidemics as well as multiple flu pandemics during the last century.

In addition to their high infectivity and virulence, efforts to prevent the spread of these viruses are complicated by their high rate of mutation. SARS-CoV-1 and SARS-CoV-2 have each mutated to produce hundreds of strains which are of further infectious concern. Influenza A viruses persistently undergo genetic reassortment, generating multiple viral subtypes (e.g., H1N1, H3N2) that concurrently circulate in human populations and those of other natural hosts.

### SUMMARY

The discovery, development and implementation of safe and effective vaccines is of ongoing importance for addressing the SARS-CoV-2 pandemic, as well as preparing for epidemics that may occur in the future, such as those caused by influenza viruses.

Immunization of distinct vulnerable populations, such as the elderly and immunocompromised, may result in sub-optimal responses, necessitating multiple booster doses and may be limited by waning immunity. Adjuvantation is a key approach to enhancing vaccine-induced immunity. Adjuvants can enhance, prolong, and modulate immune responses to vaccinal antigens to maximize protective immunity, and may enable more effective immunization of vulnerable populations (e.g., in the very young and the elderly or for diseases lacking effective vaccines). Further, the risk for SARS-CoV-2 vaccine-induced antibody disease enhancement (ADE) must also be addressed.

Some aspects of the present disclosure provide methods of inducing an immune response to a virus in a subject in need thereof, the method comprising administering to the subject a viral antigen and an adjuvantation system comprising a fungal polysaccharide.

In some embodiments, the fungal polysaccharide comprises a soluble polysaccharide. In some embodiments, the fungal polysaccharide comprises a mannan. In some embodiments, the fungal polysaccharide is isolated from Candida albicans. In some embodiments, the adjuvantation system further comprises alum. In some embodiments, the fungal polysaccharide is adsorbed into the alum. In some embodiments, the fungal polysaccharide is conjugated to the alum.

In some embodiments, the virus is a Beta coronavirus is selected from Middle East Respiratory Syndrome coronavirus (MERS-CoV), Severe Acute Respiratory Syndrome (SARS)-associated coronavirus (SARS-CoV)-1, and SARS-CoV-2.

In some embodiments, the viral antigen comprises a Beta coronavirus protein or polypeptide. In some embodiments, the viral antigen comprises a nucleic acid encoding a Beta coronavirus protein or a polypeptide. In some embodiments, the nucleic acid is DNA or RNA. In some embodiments, the RNA is a messenger RNA (mRNA). In some embodiments, the Beta coronavirus protein or polypeptide comprises a Beta coronavirus spike protein or spike protein receptor binding domain. In some embodiments, the Beta coronavirus spike protein is a MERS-CoV spike protein or spike protein receptor binding domain, SARS-CoV-1 spike protein or spike protein receptor binding domain, or SARS-CoV-2 spike protein or spike protein receptor binding domain. In some embodiments, the viral antigen comprises a viral particle of MERS-CoV, SARS-CoV-1, or SARS-CoV-2. In some embodiments, the viral antigen comprises killed or inactivated MERS-CoV, SARS-CoV-1, or SARS-CoV-2. In some embodiments, the viral antigen comprises killed or live attenuated MERS-CoV, SARS-CoV-1, or SARS-CoV-2.

In some embodiments, the virus is an influenza A or influenza B virus.

In some embodiments, the viral antigen comprises an influenza A virus or influenza B virus protein or polypeptide. In some embodiments, the viral antigen comprises a nucleic acid encoding an influenza A virus or influenza B virus protein or a polypeptide. In some embodiments, the nucleic acid is DNA or RNA. In some embodiments, the RNA is a messenger RNA (mRNA). In some embodiments, the influenza A virus or influenza B virus protein or polypeptide comprises a hemagglutinin (HA) protein, a neuraminidase (NA) protein, or polypeptide thereof. In some embodiments, the viral antigen comprises a viral particle of an influenza A virus or an influenza B virus. In some embodiments, the viral antigen comprises killed or inactivated influenza A virus or influenza B virus. In some embodiments, the viral antigen comprises killed or live attenuated influenza A virus or influenza B virus.

In some embodiments, the subject is human. In some embodiments, the subject is a human neonate, a human infant, an adult human, or an elderly human. In some embodiments, the subject is a companion animal or a research animal. In some embodiments, the subject is immune-compromised, has chronic lung disease, asthma, cardiovascular disease, cancer, obesity, diabetes, chronic kidney disease, and/or liver disease.

In some embodiments, the viral antigen and the adjuvantation system are administered simultaneously. In some embodiments, the viral antigen and the adjuvantation system are administered separately. In some embodiments, the administering is done intramuscularly, intradermally, orally, intravenously, topically, intranasally, or sublingually. In some embodiments, the administration is prophylactic.

In some embodiments, the adjuvantation system elicits a type 1 immune response in the subject. In some embodiments, the adjuvantation system promotes the activation of dendritic cell-associated C-type lectin 2 (Dectin-2) in the subject. In some embodiments, the adjuvantation system leads to an innate immune response of the subject. In some embodiments, the adjuvantation system enhances B cell immunity. In some embodiments, the adjuvantation system enhances the production of antigen-specific antibodies, compared to when the viral antigen is administered alone. In some embodiments, the adjuvantation system enhances the production of antigen-specific IgG2c antibodies. In some embodiments, the adjuvantation system enhances the cytokine production of antigen-specific T cells, compared to when the viral antigen is administered alone. In some embodiments, the adjuvantation system enhances the production of IFNγ. In some embodiments, the adjuvantation system polarizes the innate immune response toward T follicular helper (Tfh) cell immunity. In some embodiments, the adjuvantation system polarizes the innate immune response toward T helper 1 (Th1) cell immunity. In some embodiments, the adjuvantation system prolongs a protective effect in the subject against the viral antigen, compared to when the viral antigen is administered alone. In some embodiments, the adjuvantation system increases rate of an immune response, compared to when the viral antigen is administered alone. In some embodiments, the viral antigen produces a same level of immune response against the antigen at a lower dose in the presence of the adjuvantation system, compared to when the viral antigen is administered alone. In some embodiments, the likelihood of antibody disease enhancement (ADE) is reduced in the subject, compared to when the viral antigen is administered alone.

Other aspects of the present disclosure provide adjuvantation systems comprising a fungal polysaccharide for use in inducing an immune response against a virus (e.g., a Beta coronavirus, such as MERS-CoV, SARS-CoV-1, or SARS-CoV-2, or an influenza virus, such as an influenza A virus or an influenza B virus) in a subject in need thereof.

In some aspects, the present disclosure provides an adjuvantation system comprising a fungal polysaccharide and alum for use in inducing an immune response against a virus (e.g., a Beta coronavirus, such as MERS-CoV, SARS-CoV-1, or SARS-CoV-2, or an influenza virus, such as an influenza A virus or an influenza B virus) in a subject in need thereof.

Also provided herein are immunogenic compositions comprising a viral antigen and an adjuvantation system comprising a fungal polysaccharide. In some embodiments, the fungal polysaccharide comprises mannan. In some embodiments, the fungal polysaccharide comprises a fungal polysaccharide isolated from Candida albicans. In some embodiments, the adjuvantation system further comprises alum. In some embodiments, the fungal polysaccharide is adsorbed into the alum. In some embodiments, the fungal polysaccharide is conjugated to the alum.

In some embodiments, the virus is a Beta coronavirus selected from Middle East Respiratory Syndrome coronavirus (MERS-CoV), Severe Acute Respiratory Syndrome (SARS)-associated coronavirus (SARS-CoV)-1, and SARS-CoV-2. In some embodiments, the viral antigen comprises a Beta coronavirus protein or polypeptide. In some embodiments, the viral antigen comprises a nucleic acid encoding a Beta coronavirus protein or a polypeptide. In some embodiments, the nucleic acid is DNA or RNA. In some embodiments, the RNA is a messenger RNA (mRNA). In some embodiments, the Beta coronavirus protein or polypeptide comprises a Beta coronavirus spike protein or spike protein receptor binding domain. In some embodiments, the Beta coronavirus spike protein is a MERS-CoV spike protein or spike protein receptor binding domain, SARS-CoV-1 spike protein or spike protein receptor binding domain, or SARS-CoV-2 spike protein or spike protein receptor binding domain. In some embodiments, the viral antigen comprises a viral particle of MERS-CoV, SARS-CoV-1, or SARS-CoV-2. In some embodiments, the viral antigen comprises killed or inactivated MERS-CoV, SARS-CoV-1, or SARS-CoV-2. In some embodiments, the viral antigen comprises killed or live attenuated MERS-CoV, SARS-CoV-1, or SARS-CoV-2.

In some embodiments, the virus is an influenza virus selected from an influenza A virus and an influenza B virus. In some embodiments, the viral antigen comprises an influenza A virus or an influenza B virus protein or polypeptide. In some embodiments, the viral antigen comprises a nucleic acid encoding an influenza A virus or an influenza B virus protein or a polypeptide. In some embodiments, the nucleic acid is DNA or RNA. In some embodiments, the RNA is a messenger RNA (mRNA). In some embodiments, the influenza A virus or an influenza B virus protein or polypeptide comprises a hemagglutinin (HA) protein, a neuraminidase (NA) protein, or polypeptide thereof. In some embodiments, the viral antigen comprises a viral particle of an influenza A virus or an influenza B virus. In some embodiments, the viral antigen comprises killed or inactivated influenza A virus or influenza B virus. In some embodiments, the viral antigen comprises killed or live attenuated influenza A virus or influenza B virus.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1K. Mannans elicit lymph node-restricted IFN signatures that drive lymph node expansion. FIG. 1A: Mice were injected intradermally with saline (Sal.), mannans (Mann.) or β-glucans (β-gluc.). 24 hours later the injection site was assessed for the presence of an abscess with or without skin lesion. The graph depicts percentages of mice in each of the indicated categories. Representative pictures of skin appearance at injection sites of saline, mannans and β-glucans are also shown. N=5 mice per group. FIG. 1B: Transcriptomic analysis of skin samples collected 6 hours after injection of saline (Sal.), β-glucans (β-gluc.) or mannans (Mann.). Heatmap of abundance (z-scored log 2 normalized counts) of genes induced by β-glucans and/or mannans compared to saline control, ranked by abundance difference between β-glucans and mannans. The gap splits the genes into two clusters, one that is highly upregulated by β-glucans and one that is highly upregulated by mannans. N=3 mice per group. FIG. 1C: Mice were injected intradermally with saline, mannans (Mann.) or β-glucans (β-gluc.). 6 or 24 hours later dLNs were collected and analyzed for weight as well as absolute numbers of CD45⁺, B and T cells. Results are expressed as fold over contralateral, saline-injected LN. N=5-9 mice per group. FIG. 1D: Mice were injected intravenously with a blocking anti-CD62L antibody (αCD62L) or the same dose of an isotype control (Iso CTRL) one day before intradermal injections of saline or mannans. 24 hours later dLNs were collected and their weights were measured. Results are expressed as fold over contralateral, saline-injected LN. N=4 mice per group. FIG. 1E: WT and Ccr7^{-/-} mice were intradermally injected with saline (Sal.) or fluorescently labelled mannans (Mann.). 1, 6 and 24 hours later dLNs were collected and homogenized to measure total fluorescence. Results are expressed as arbitrary units (A.U.) of fluorescence and shown as mean+SEM. N=3 mice per timepoint. FIG. 1F: Ccr7^{-/-} mice were injected intradermally with saline or mannans. 24 hours later dLNs were collected and analyzed as indicated in FIG. 1C. N=6 mice. FIG. 1G: Transcriptomic analysis of dLNs collected 6 and 24 hours after intradermal injection of saline (Sal.), β-glucans (β-gluc.) or mannans (Mann.). Heatmap of abundance (z-scored log 2 normalized counts) of genes induced by β-glucans and/or mannans compared to saline control, ranked by abundance difference between β-glucans and mannans. The gap splits the genes into two clusters, one that is highly upregulated by β-glucans and one that is highly upregulated by mannans. N=4-5 mice per group. FIG. 1H: Pathway enrichment analysis of genes belonging to the cluster upregulated by mannans as depicted in FIG. 1G. FIG. 1I: Heatmap representation of the average expression levels of the top 50 genes upregulated in mannan-treated dLNs 24 hours after the injection compared to the saline control. N=4-5 mice per group. FIG. 1J: WT and Ifnar^{-/-} mice were intravenously injected with an anti-IFNγ blocking antibody (aIFNγ) or the same dose of an isotype control (Iso CTRL) on day −1 and 0. On day 0 mice were also intradermally injected with saline (Sal.) or mannans (Mann.). 24 hours later dLNs were collected, their weights were measured, and RNA was extracted for gene expression analysis. Results are expressed as fold over contralateral, saline-injected LN (weight) or as relative expression compared to Gapdh. N=4 mice per group. FIG. 1K: WT and Ifnar^{-/-} Ifngr^{-/-} mice were intradermally injected with saline (Sal.) or Lipo-CpG. Samples were collected and analyzed as in FIG. 1J. N=5 mice per group. #and ##respectively indicate p≤0.05 and 0.01 when comparing each group against the value 1 (which represent the contralateral control sample expressed as fold). * and ** respectively indicate p≤0.05 and 0.01 when comparing among different experimental groups.

FIGS. 2A-2M. The mannan-elicited lymph node innate response requires Dectin-2-expressing, CD169⁺ sinus macrophages. FIG. 2A: WT, Clec4n^{-/-} and Fcer1g^{-/-} mice were intradermally injected with saline or mannans. 24 hours later dLNs were collected, their weights were measured and RNA was extracted for gene expression analysis. Results are expressed as fold over contralateral, saline-injected LN. N=3-5 mice per genotype. FIG. 2B: WT mice were intradermally injected with fluorescently labelled mannans (Mann.-AF488). 6 hours later dLNs were collected and the absolute numbers of mannan-laden (Mann.⁺) CD3/CD19/NK1.1⁻ cells were quantified by flow cytometry. N=6 mice. FIG. 2C: Mice were treated as in FIG. 2B. Imaging cytometry analysis and quantification of mannan internalization was performed on CD3/CD19/NK1.1-depleted, CD45⁺ mannan-laden (Mann.⁺) cells. N=4 mice. FIG. 2D: WT mice were intradermally injected with fluorescently labelled mannans (Mann.). 1 hour later dLNs were collected for confocal microscopy analysis using antibodies against B220 and phospho-Syk (pSyk). DAPI was used for nuclear counterstaining. One representative image is shown. FIG. 2E: WT and Fcer1g^{-/-} mice were injected with saline or fluorescently labelled mannans. 6 hours later dLNs were collected and CD86 expression levels were assessed by flow cytometry on CD3/CD19/NK1.1⁻ CD45⁺ mannan-laden (Mann.⁺) cells, CD45⁺ cells that did not capture mannans (Mann.⁻) and CD45⁺ cells from saline-injected dLNs (Sal.). N=6 mice per genotype. FIG. 2F: WT mice were intradermally injected with fluorescently labelled mannans. 6 hours later dLNs were collected and the phenotype of CD3/CD19/NK1.1⁻ CD45⁺ mannan-laden (Mann.⁺) cells was assessed by flow cytometry. N=6 mice. FIGS. 2G-2I: DT-treated CD11c-DTR, Ccr2^{-/-} and isotype control (Iso CTRL)- or anti-Ly6G (αLy6G)-treated mice were treated and analyzed as in FIG. 2A. N=4 mice per group. FIGS. 2J, 2K: LNs were isolated from untreated WT mice and the expression of Dectin-2 (D2) was evaluated by flow cytometry as percentage of expression in the indicated CD3/CD19/NK1.1⁻ CD45⁺ cell subsets. N=6 for FIG. 2J or 3 for FIG. 2K. FIG. 2L: Confocal microscopy analysis of untreated LNs stained with antibodies against Dectin-2 (D2), B220 and CD169. DAPI was used for nuclear counterstaining. 1 representative image is shown. FIG. 2M: DT-treated CD169-DTR mice were treated and analyzed as in FIG. 2A. N=4 mice per group. #and ##respectively indicate p≤0.05 and 0.01 when comparing each group against the value 1 (which represent the contralateral control sample expressed as fold) or saline control. * and ** respectively indicate p≤0.05 and 0.01 when comparing among different experimental groups.

FIGS. 3A-3D. Activation of the non-canonical NF-kB subunit RelB governs the mannan-elicited lymph node innate response. FIG. 3A: WT and Card9^{-/-} mice were treated and analyzed as in FIG. 2A. N=9 (for LN weight) or 4 (for gene expression analysis) mice per genotype. FIG. 3B: CD3⁻ CD19⁻ NK1.1⁻ Ter119⁻ CD45⁺ AF488-mannan⁺ Ly6G⁻ (CD11b⁺ Ly6C⁺)⁻ CD11b⁺ CD11c⁺ cells were sorted from dLNs of WT, Fcer1g^{-/-} and Card9^{-/-} mice 6 hours after AF488-mannan injection and transcriptional profiles were assessed by targeted transcriptome sequencing. Results are shown as heatmap of genes with an F-test FDR less than 0.05 and a log 2 fold-change (FC) greater than 1 (or lower than −1) between a mutant and WT control. FIGS. 3C, 3D: Relb^{fl/fl} and Cd11c^{cre} Relb^{fl/fl} mice were treated with saline, mannans or Lipo-CpG, and analyzed as in FIG. 2A. N=4-13 mice per genotype. #and ##respectively indicate p≤0.05 and 0.01 when comparing each group against the value 1 (which represent the contralateral control sample expressed as fold) or saline control. * and ** respectively indicate p≤0.05 and 0.01 when comparing among different experimental groups.

FIGS. 4A-4H. Molecular pathways required for mannan-elicited lymph node innate response regulate the magnitude of mannan adjuvant activity. CFSE-labelled OT-II (FIGS. 4A-4D) or OT-I (FIGS. 4E-4H) CD4⁺ T cells were injected intravenously in WT mice on day −1. On day 0 the mice were intradermally injected with saline, ovalbumin (OVA), or OVA combined with mannans (Mann). 3 days later dLNs were isolated and the absolute numbers of CFSE^{lo} cells (i.e., cells that underwent at least one cycle of cell division) (FIGS. 4A, 4E) or the percentages of cells in each division peak (FIGS. 4B, 4F) were quantified by flow cytometry. N=4 mice per group. ##indicates p≤0.01 when comparing each group against saline control (FIGS. 4A, 4E). * and ** respectively indicate p≤0.05 and p≤0.01 when comparing OVA vs OVA+mann. (FIGS. 4A, 4B, 4E, 4F). FIGS. 4C, 4D, 4G, 4H: WT, Fcer1g^{-/-} and Card9^{-/-} mice were treated and analyzed as in FIGS. 4A, 4B, 4E, and 4F (with the exception that all mice received OVA combined with mannans). N=4 mice per genotype. #and ##respectively indicate p≤0.05 and p≤0.01 when comparing WT vs Fcerlg^{-/-} (black) or Card9^{-/-} vs Fcer1g^{-/-} (blue). * and ** respectively indicate p≤0.05 and 0.01 when comparing WT vs Card9^{-/-}. Results in FIGS. 4B and 4F and FIGS. 4D and 4H are shown as mean+SD.

FIGS. 5A-5L. Mannans formulated with aluminum hydroxide acquire novel physical properties that predict Immunological functions. FIG. 5A: GM-CSF differentiated, bone marrow-derived phagocytes were generated from WT and stimulated with LPS, curdlan, β-glucans (β-gluc.), mannans (Mann.), aluminum hydroxide/mannans (AlumOH/mann.). After 18-21 hours supernatants were collected, and TNF and IL-2 protein concentrations were measured by ELISA while cells were harvested and expression levels of CD86 and OX40L were measured by flow cytometry. N=5 independent experiments. FIG. 5B: Mice were intradermally injected with saline (sal.), aluminum hydroxide (AH), β-glucans (β-gluc), mannans (Mann) or aluminum hydroxide/mannans (AH/mann). 24 hours later skin samples were collected and RNA was extracted for gene expression analysis. Results are expressed as fold over contralateral, saline-injected skin sample. N=4-5 mice per group. FIG. 5C: Mice were intradermally injected with saline (Sal.), fluorescently labelled β-glucans (β-gluc), fluorescently labelled mannans (Mann) or their formulation with aluminum hydroxide (AH/mann). 24 hours later dLNs were collected and homogenized to measure total fluorescence. Results are expressed as arbitrary units (A.U.) of fluorescence and shown as individual data points (horizontal bars represent means). N=3 mice. (FIG. 5D) Mice were treated as in FIG. 5A. 1, 7 and 14 days later dLNs were collected, their weights were measured and expressed as fold over contralateral, saline-injected LN. Results are represented as mean+SEM (left panel) or area under the curve (AUC, right panel). (FIG. 5E) Mice were treated as in FIG. 5A. 24 hours later dLNs were collected, and RNA was extracted for gene expression analysis. Results are expressed as fold over contralateral, saline-injected LN. N=5 per group. (FIG. 5F) Ifnar−/− and WT mice were respectively treated with a blocking anti-IFNγ antibody (αIFNγ) or the same dose of an isotype control (Iso CTRL) on day −1 and 0, and on day 0 mice were intradermally injected with saline (sal.), β-glucans (β-gluc), or AH/mannans (AH/mann). 24 hours later dLNs were collected and their weights were measured. Results are expressed as fold over contralateral, saline-injected LN. N=5 mice per group. (FIG. 5G), WT mice treated with blocking anti-IFNAR plus anti-IFNγ (αIFNAR/IFNγ) antibodies or the same doses of isotype controls (Iso CTRL) on day −1 and 0, and on day 0 mice were intradermally injected with saline (sal.) or AH/mannans (AH/mann). 24 hours later dLNs were collected and their weights were measured. Results are expressed as fold over contralateral, saline-injected LN. N=5 mice per group. (FIGS. 5H, 5I) Mice were treated with the soluble (Sol) or particulate (Part) fractions of AH/mannans. 24 hours later skin samples (FIG. 5H) and dLNs (FIG. 5I) were collected, dLN weights were measured and RNA was extracted for gene expression analysis. Results are expressed as fold over contralateral, saline-injected skin sample or LN. N=5 mice per group. (FIG. 5J) Mice of the indicated backgrounds were injected with saline (sal.) or AH/mannans (AH/mann). 24 hours later dLNs were collected, their weights were measured, and RNA was extracted for gene expression analysis. Results are expressed as fold over contralateral, saline-injected sample or as relative expression compared to Gapdh. (FIGS. 5K, 5L) WT mice injected on day −1 and 0 with the same volumes of PBS or a depleting anti-Asialo GM1 antibody (αAsGM1) (FIG. 5K), or WT and Batf3−/− mice (FIG. 5L) were injected intradermally on day 0 with saline (Sal) or AH/mannans (AH/mann). 24 hours later dLNs were collected, and RNA was extracted for gene expression analysis. Results are reported as relative expression compared to Gapdh. N=5 mice per group. #and ##respectively indicate p≤0.05 and 0.01 when comparing each group against its untreated control (CTRL) or the value 1 (which represent the contralateral control sample expressed as fold). * and ** respectively indicate p≤0.05 and 0.01 when comparing among different experimental groups.

FIGS. 6A-6H. Immunization with SARS-CoV-2 Spike protein and aluminum hydroxide/mannans generates anti-Spike type 1 immunity and neutralizing antibodies. FIGS. 6A-6E: Mice were injected intradermally with saline (Sal), pre-fusion stabilized SARS-CoV-2 trimer alone (S) or combined with AH (S/AH), β-glucans (S/β-gluc.), mannans (S/mann) or AH/mannans (S/AH/mann) on day 0 (prime) and day 14 (boost). Serum samples were collected on day 28 to assess anti-Spike (FIG. 6A) and anti-RBD (FIG. 6B) antibody levels, SARS-CoV-2 surrogate virus neutralization test (FIG. 6D) and neutralization titer (FIG. 6E). In selected experiments (FIG. 6C), mice were sacrificed on day 35 to collect spleens and isolate splenocytes for in vitro restimulation with Spike peptides. After 96 hours supernatants were collected and IFNg protein levels were measured by ELISA. N=16-18 (FIGS. 6A, 6B), 10 (FIG. 6C), 8-10 (FIG. 6D) or 13-15 (FIG. 6E) mice per group. FIGS. 6F-6H: Mice were injected intradermally with saline (Sal), pre-fusion stabilized SARS-CoV-2 trimer alone (S), or combined with AH (S/AH). Mannans (Mann) were injected separately on the same side of the S/AH injection in a proximal site, either the same day (S/AH+Mann (D 0)) or the day before (S/AH+Mann (D−1)). As a control, SARS-CoV-2 trimer combined with AH and mannans (S/AH/Mann) was also injected. Formulations were injected on day 0 (prime) and day 14 (boost). Serum samples were collected on day 28 to assess anti-Spike antibody levels (FIG. 6F) and SARS-CoV-2 neutralization titer (FIG. 6G). In selected experiments (FIG. 6H), mice were sacrificed on day 35 to collect spleens and isolate splenocytes for in vitro restimulation as in C. N=6-8 mice per group. #, * and ##, ** respectively indicate p≤0.05 and 0.01 when comparing among different experimental groups. Comparisons are indicated by the shade. Comparisons are indicated by the shade.

FIG. 7. Immunization with SARS-CoV-2 Spike protein and aluminum hydroxide/mannans generates cross-reactive anti-Spike antibodies with broad epitope specificity. Mice were immunized as in FIGS. 6A-6E. VirScan analysis was performed on serum samples collected on day 28. Each column represents a single serum sample collected from an individual mouse and each row represents a peptide tile. Tiles are labeled by amino acid start and end position. Shade intensity represents the degree of enrichment (z-score) of each peptide. Shaded lines indicate the approximate aminoacidic positions (AA pos.) of RBD, Fusion peptides and Heptad repeat 2 of each virus. N=6 mice per group.

FIGS. 8A-8F. The AH/mannan adjuvant formulation confers protection against lung viral infections. FIGS. 8A, 8B: Mice were injected intradermally with saline (Sal), pre-fusion stabilized SARS-CoV-2 trimer alone (S) or combined with AH (S/AH), AH/mannans (S/AH/mann), AddaS03 (S/AddaS03), or AH/PHAD (S/AH/PHAD) on day 0 (prime) and day 14 (boost). Serum samples were collected on day 28 to assess anti-Spike and anti-RBD antibody levels (FIG. 8A). On day 35 mice were intranasally infected with SARS-CoV-2 MA10 on day 35 and 2 days later numbers of plaque forming units (PFU) were quantified in the lungs (FIG. 8B). N=4-5 mice per group. FIGS. 8C-8F: Mice were injected intradermally with saline (Sal), Flublok alone (rHA) or combined with AH (rHA/AH), AH/mannans (rHA/AH/mann), AddaVax (rHA/AddaVax), or AH/PHAD (rHA/AH/PHAD) on day 0 (prime) and day 14 (boost). Serum samples were collected on day 28 to assess antibodies against rHA (anti-rHA, C) or IAV A/PR/8/1934 recombinant hemagglutinin (anti-rPR8, FIG. 8E). On day 35 mice were intranasally infected with IAV A/PR/8/1934 and body weights were recorded for 7 days (FIG. 8D). N=5 (FIGS. 8C, 8E) or 8 (FIG. 8D) mice per group. On day 7 post-infection mice were sacrificed and lungs were collected for histological analysis (hematoxylin eosin staining, FIG. 8F). One representative image per group is shown. #, * and ##, ** respectively indicate p≤0.05 and 0.01. Comparisons are indicated by the shade.

FIG. 9. Mannans and β-glucans exhibit different diameters. Hydrodynamic diameters of mannan (Mann.) and β-glucan (β-gluc.) preparations were measured by dynamic light scattering. Results from 1 representative experiment are shown.

FIGS. 10A-10B. Soluble mannans are inactive in vitro. GM-CSF differentiated, bone marrow-derived phagocytes were generated from WT (FIGS. 10A, 10B), Clec7a^{-/-}, Clec4n^{-/-} and Fcer1g^{-/-} mice (FIG. 10B) and stimulated with LPS, curdlan, β-glucans (β-gluc), soluble mannans (Mann) (FIGS. 10A, 10B), uncoated microbeads (B only) and microbeads covalently linked to mannans (B:Mann) (FIG. 10B). After 18-21 hours supernatants were collected, and TNF and IL-2 concentrations were measured by ELISA while cells were harvested and expression levels of CD86 and OX40L were measured by flow cytometry. N=4-6 independent experiments. ##indicate p≤0.01 when comparing each group against its untreated control (CTRL). Results are shown as mean+SEM.

FIG. 11. Pathway enrichment analysis of genes belonging to the cluster upregulated by β-glucans as depicted in FIG. 1B.

FIGS. 12A-12N. Mannans and β-glucans induce unique patterns of immune cell recruitment and activation in the draining LNs. FIGS. 12A-12H: Mice were injected intradermally with saline (black plots), mannans (Mann, red plots) or β-glucans (β-gluc, red plots). 6 or 24 hours later dLNs were collected and analyzed by flow cytometry for absolute numbers (FIGS. 12A-12G) or CD86 expression (FIG. 12H) of the indicated populations. N=5-6 mice per group. FIGS. 12I-12K: Mice were injected intradermally with saline (Sal) or mannans (Mann). 24 hours later dLNs were collected and analyzed for absolute numbers of total IFNγ⁺ cells (FIG. 12I) or the indicated cell populations among CD45⁺ cells (FIG. 12J). Levels of IFNγ production in CD8⁺ T cells or NK cells were evaluated by measuring the MF of IFNγ among IFNγ-expressing cells (FIG. 12K). FIGS. 12L-12N: WT mice treated on day −1 and 0 with the same volumes of PBS or a depleting anti-Asialo GM1 antibody (aAsGM1) (FIGS. 12L, 12N), or WT and Batf3^{-/-} mice (FIG. 12M) were injected intradermally on day 0 with saline or mannans. 24 hours later dLNs were collected and RNA was extracted for gene expression analysis (FIGS. 12L, 12M), or dLNs were weighted and analyzed by flow cytometry for absolute numbers of the indicated populations (FIG. 12N). Results are expressed as fold over contralateral, saline-injected LN. N=4-5 mice per group. #and ##respectively indicate p≤0.05 and 0.01 when comparing between treatments (Mann vs β-gluc, FIGS. 12A-12H) or each group against the value 1 (which represent the contralateral control sample expressed as fold, FIGS. 12L, 12M). * and ** respectively indicate p≤0.05 and 0.01 when comparing between treatments (Mann, β-gluc) and their respective saline controls (FIGS. 12A-12H) or between the indicated experimental groups (FIGS. 12K, 12L).

FIGS. 13A-13B. Soluble whole glucan particles elicit LN expansion and ISG expression. Mice were injected intradermally with saline (Sal.), dispersible (WGP-D) or soluble (WGP-S) whole glucan particles. 24 hours later injection sites were assessed as indicated in FIG. 1A. FIG. 13A: skin samples and dLNs were collected, LN weights were measured and RNA extracted for gene expression analysis. FIG. 13B: Results are expressed as fold over the median value of saline-injected skin samples or LNs. N=4 mice per group. #and ##respectively indicate p≤0.05 and 0.01 when comparing each group against the value 1 (which represent the saline control samples expressed as fold). * and ** respectively indicate p≤0.05 and 0.01 when comparing among different experimental groups.

FIG. 14. Mannans elicit CARD-9-independent responses in the dLN. Pathway analysis of genes significantly induced in WT compared to Fcer1g^{-/-} mice as depicted in FIG. 3B.

FIGS. 15A-15I. Mannans formulated with aluminum hydroxide acquire novel physical properties that predict immunological functions. FIG. 15A: Soluble mannans (Mann), AH and the formulation of mannans and AH (AH/mann) were incubated at room temperature for 30 minutes, then spun down and the supernatants were collected for 1H-nuclear magnetic resonance quantification of unbound mannans. The reaction contained an excess of mannan. The results show that the mannan absorption capacity of AH is approximately two times its mass in this formulation strategy. Results are expressed as percentage of soluble mannans and shown as mean+SD. FIGS. 15B-15I: GM-CSF differentiated, bone marrow-derived phagocytes were generated from WT or the indicated knock out mice and stimulated with AH, mannans (Mann), AH/mannans (AH/mann). After 18-21 hours supernatants were collected, and TNF and IL-2 protein concentrations were measured by ELISA (FIGS. 15B, 15C). Cells were harvested and expression levels of CD86 and OX40L were measured by flow cytometry (FIGS. 15D, 15E). Alternatively, cells were stimulated for 6 hours, and RNA was extracted for gene expression analysis. Results are reported as relative expression compared to Rpl13a (FIGS. 15F-15I). N=3 (FIG. 15A) or 4 (FIGS. 15B, 15I) independent experiments. #and ##respectively indicate p≤0.05 and 0.01 when comparing the same genotype across treatments. * and ** respectively indicate p≤0.05 and 0.01 when comparing the same treatment across genotypes. Comparisons are indicated by the shade.

FIGS. 16A-16G. B and T cell responses activated by immunization with SARS-CoV-2 Spike formulated with AH/mannans have the same cellular and molecular requirements of the LIR elicited by AH/mannans. WT mice, transgenic mice or WT mice treated on day −1, 0, 13 and 14 with blocking anti-IFNAR plus anti-IFNγ (αIFNAR/IFNγ) antibodies or the same doses of isotype controls (Iso CTRL), were injected intradermally with pre-fusion stabilized SARS-CoV-2 trimer combined with AH (S/AH) or AH/mannans (S/AH/mann) on day 0 (prime) and day 14 (boost). Serum samples were collected on day 28 (FIGS. 16B-16G) or on day 98 (FIG. 16A, WT mice) to assess anti-Spike antibody levels. In selected experiments (FIGS. 16B, 16C), mice were sacrificed on day 35 to collect spleens and isolate splenocytes for in vitro restimulation with Spike peptides. After 96 hours supernatants were collected and IFNγ protein levels were measured by ELISA. N=4-7 mice per group. #and ##respectively indicate p≤0.05 and 0.01 when comparing S/AH and S/AH/mann vs Sal (FIG. 16A) or S/AH/mann vs S/AH (FIGS. 16B-16G). * and ** respectively indicate p≤0.05 and 0.01 when comparing S/AH/mann vs S/AH (FIG. 16A) or S/AH/mann across treatments or genotypes (FIGS. 16B-16G). Comparisons are indicated by the shade.

FIGS. 17A-17D. The AH/mann adjuvant formulation confers protection against lung viral infections. FIGS. 17A, 17B: Mice were injected intradermally with saline (Sal), pre-fusion stabilized SARS-CoV-2 trimer alone (S) or combined with AH (S/AH), AH/mannans (S/AH/mann), AddaS03 (S/AddaS03), or AH/PHAD (S/AH/PHAD) on day 0 (prime) and day 14 (boost). Serum samples were collected on day 28 to assess SARS-CoV-2 surrogate virus neutralization test (FIG. 17A) and neutralization titer (FIG. 17B). N=4-5 mice per group. FIGS. 17C, 17D: Mice were injected intradermally with saline (Sal), Flublok alone (rHA) or combined with AH (rHA/AH), AH/mannans (rHA/AH/mann), AddaVax (rHA/AddaVax), or AH/PHAD (rHA/AH/PHAD) on day 0 (prime) and day 14 (boost). On day 35 mice were intranasally infected with IAV A/PR/8/1934. On day 42 serum samples were collected to assess anti-IAV A/PR/8/1934 recombinant hemagglutinin (Anti-rPR8) levels (FIG. 17C). Histology score of lung images collected as in in FIG. 8F (FIG. 17D). N=5 mice per group. #and ##respectively indicate p≤0.05 and 0.01. Comparisons are indicated by the shade.

### DETAILED DESCRIPTION OF CERTAIN EMBODIMENTS

Human immunity is crucial to both health and illness, playing key roles in multiple major diseases including infectious diseases, allergies, and cancer. Infectious diseases are a leading cause of morbidity and mortality at the extremes of life. SARS-coronavirus-2 (SARS-CoV-2), the causal agent of COVID-19, first emerged in late 2019 in China. It has infected over 115 million individuals and caused>2.5 million deaths globally, particularly among elderly populations. And yet, even during the SARS-CoV-2 pandemic, numerous other viruses continue to be of major concern to human health, including influenza viruses Discovery, development, and implementation of safe and effective vaccines will be key to addressing the both the current SARS-CoV-2 pandemic, as well as other epidemics that may occur in the future.

Immunization of distinct vulnerable populations such as the elderly and immunocompromised may result in sub-optimal responses, often requiring multiple booster doses, and may be limited by waning immunity. Adjuvantation is a key approach to enhance vaccine-induced immunity. Adjuvants can enhance, prolong, and modulate immune responses to vaccinal antigens to maximize protective immunity, and may potentially enable effective immunization in vulnerable populations (e.g., in the very young and the elderly or for diseases lacking effective vaccines). Further, theoretical risk of SARS-CoV-2 vaccine-induced antibody disease enhancement (ADE) also needs to be addressed.

Some aspects of the present disclosure provide immunogenic compositions (e.g., vaccine compositions) comprising a viral antigen and an adjuvantation system comprising a polysaccharide derived from a fungal species. In some embodiments, the adjuvantation system further comprises alum (e.g., the fungal polysaccharide is formulated with alum). In some embodiments, the fungal polysaccharide (e.g., a mannan) is adsorbed in alum. In some embodiments, the fungal polysaccharide (e.g., a mannan) is covalently conjugated to alum. The immunogenic composition (e.g., vaccine composition) provide herein may be used in methods of inducing an immune response to an antigen in a subject in need thereof, the method comprising administering to the subject an effective amount of a viral antigen and an effective amount of the adjuvantation system (e.g., either comprising a fungal polysaccharide alone, or comprising a fungal polysaccharide and alum). In some embodiments, the immunogenic composition (e.g., vaccine composition) described herein may be used for inducing an immune response in a subject that is a newborn, an adult, or an elderly human (e.g., older than 65 years old).

“Beta coronavirus” is one of four genera (Alpha-, Beta-, Gamma-, and Delta-) of coronaviruses. Beta coronaviruses belong to the subfamily Orthocoronavirinae in the family Coronaviridae, of the order Nidovirales. They are enveloped, positive-sense, single-stranded RNA viruses of zoonotic origin. Beta coronaviruses of the greatest clinical significance to humans include SARS-CoV-1 (which causes SARS), SARS-CoV-2 (which causes the disease COVID-19), and MERS-CoV (which causes MERS).

“Influenza virus” refers to one of the four types of influenza viruses: influenza A virus (IAV), influenza B virus (IBV), influenza C virus, and influenza D virus. Influenza viruses belong to the family Orthomyxoviridae in the order Articulavirales. All except for influenza D are known to cause disease in humans, while influenza A and influenza B viruses are of the greatest risk of becoming epidemic and are therefore of the greatest clinical significance. Influenza viruses are frequently categorized according to their subtype. Influenza A viruses in particular are categorized according to which variants of hemagglutinin protein (HA) and neuraminidase protein (NA) they encode. There are 18 distinct hemagglutinin variants (i.e., H1 through H18) and 11 different neuraminidase variants (i.e., N1 through N11), for a theoretical total of 198 possible influenza A subtypes. Strains of influenza A and influenza may be further categorized according to clades and subclades. Influenza A subtypes of particular clinical significance include A/H1N1 (1918 “Spanish flu” and 2009 swine flu), A/H5N1 (2008 avian flu), and A/H7N9 (2013 avian flu). Influenza B subtypes of particular clinical significance include B/Victoria and B/Yamagata.

A “fungal polysaccharide” is a polymer of carbohydrates (e.g., sugars) that are synthesized by any cellular species belonging to the kingdom Fungi. A fungal polysaccharide may be produced and secreted by a fungal cell or occur as a component of a fungal cell (e.g., as a structural component of a fungal cell wall). A fungal polysaccharide may be covalently conjugated to another chemical moiety, such as but not limited to a protein (e.g., a glycoprotein expressed on the surface of a fungal cell). A fungal polysaccharide may be composed of one or more than one type of carbohydrate monomer covalently linked in such a way as to form a polysaccharide. A fungal polysaccharide may be soluble, partially soluble, or insoluble in solution, particularly in an aqueous solution. A fungal polysaccharide may be measured according its length (diameter) and may have a diameter of 1 nm or more, 5 nm or more, 10 nm or more, 15 nm or more, 20 nm or more, 25 nm or more, 50 nm or more, 75 nm or more, 100 nm or more, 200 nm or more, 300 nm or more, 400 nm or more, 500 nm or more, 600 nm or more, 700 nm or more, 800 nm or more, 900 nm or more, or 1000 nm or more. A fungal polysaccharide may be homogenous or heterogenous in length, such that at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% or polysaccharides have approximately the same diameter. A fungal polysaccharide may be a fungal oligosaccharide. A fungal polysaccharide may be a derivative fungal polysaccharide, such as, for instance, a shortened (i.e., lower molecular weight) or elongated (i.e., higher molecular weight) version of a polysaccharide originally isolated from a fungal cell. A fungal polysaccharide may have antigenic properties (i.e., activates an immune response in an animal or human subject). A fungal polysaccharide may be a ligand for an antigen-specific receptor of cells of an animal or human subject.

In some embodiments, the fungal polysaccharide for use in the immunogenic composition (e.g., vaccine composition) and methods described herein is a mannan (i.e., a mannose polymer). In some embodiments, the fungal polysaccharide for use in the immunogenic composition (e.g., vaccine composition) and methods described herein is a β-glucan. In some embodiments, the fungal polysaccharide for use in the immunogenic composition (e.g., vaccine composition) and methods described herein elicits an immune response in a subject. In some embodiments, the fungal polysaccharide for use in the immunogenic composition (e.g., vaccine composition) and methods described herein is isolated from a pathogenic fungus that elicits an immune response, such as Candida albicans. The effects of fungal polysaccharides (e.g., mannans, β-glucans, etc.) as vaccine adjuvants in immunization against Beta coronaviruses, especially SARS-CoV-1 and SARS-CoV-2, or influenza viruses have not previously been investigated or demonstrated.

An “adjuvantation system” refers to a composition comprising one or more adjuvants. An “adjuvant” refers to a pharmacological or immunological agent that modifies the effect of other agents, for example, of an antigen in a vaccine. Adjuvants are typically included in vaccines to enhance the recipient subject's immune response to an antigen. The use of adjuvants allows the induction of a greater immune response in a subject with the same dose of antigen, or the induction of a similar level of immune response with a lower dose of injected antigen. Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system see, e.g., H. S. Warren et al, Annu. Rev. Immunol., 4:369 (1986), incorporated herein by reference. The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (e.g., aluminum hydroxide, aluminum phosphate, aluminum potassium sulfate, etc., collectively referred to herein as “alum”), liposomes, lipopolysaccharide (LPS) or derivatives thereof such as monophosphoryl lipid A (MPLA) and glycopyranosyl lipid A (GLA), molecular cages for antigen, components of bacterial cell walls, endocytosed nucleic acids such as double-stranded RNA (dsRNA), single-stranded DNA (ssDNA), and unmethylated CpG dinucleotide-containing DNA. Typical adjuvants include water and oil emulsions, e.g., Freund's adjuvant and MF59, and chemical compounds such as alum. At present, currently licensed vaccines in the United States contain only a limited number of adjuvants, such as alum which enhances production of T helper type 2 (Th2) cells, and MPLA which activates innate immunity via Toll-like receptor 4 (TLR4). Many of the most effective adjuvants include bacteria or their products, e.g., microorganisms such as the attenuated strain of Mycobacterium bovis, Bacille Calmette-Guérin (BCG); microorganism components, e.g., alum-precipitated diphtheria toxoid, bacterial lipopolysaccharides (“endotoxins”) and their derivatives such as MPLA and GLA.

In some embodiments, the adjuvantation system of the present disclosure comprises a fungal polysaccharide (e.g., a mannan). In some embodiments, the adjuvantation system of the present disclosure comprises a fungal polysaccharide (e.g., a mannan) and aluminum salts (referred to herein as “alum”). In some embodiments, the alum is Alhydrogel® (InvivoGen, USA). In some embodiments, in a adjuvantation system comprising fungal polysaccharide (e.g., a mannan) and alum, the fungal polysaccharide (e.g., a mannan) is adsorbed into alum (e.g., as described in Jones et al., Journal of Biological Chemistry 280, 13406-13414, 2005, incorporated herein by reference). In some embodiments, in a adjuvantation system comprising fungal polysaccharide (e.g., a mannan), the fungal polysaccharide (e.g., a mannan) is covalently conjugated to an adjuvant, such as alum, a liposome, or lipopolysaccharide (LPS) or derivatives thereof such as monophosphoryl lipid A (MPLA) and glycopyranosyl lipid A (GLA).

Adjuvants or adjuvantation systems are used in immunogenic composition (e.g., the virus immunogenic composition (e.g., vaccine composition) described herein). The terms “vaccine composition” and “vaccine” are used interchangeably herein. An “immunogenic composition” is a composition that activates or enhances a subject's immune response to an antigen after the vaccine is administered to the subject. Vaccine compositions are a type of immunogenic compositions. In some embodiments, an immunogenic composition stimulates the subject's immune system to recognize the antigen (e.g., a Beta coronavirus antigen, an influenza virus antigen) as foreign, and enhances the subject's immune response if the subject is later exposed to a pathogen (e.g., Beta coronavirus, influenza virus), whether attenuated, inactivated, killed, or not. Vaccines may be prophylactic, for example, preventing or ameliorating a detrimental effect of a future exposure to a pathogen (e.g., Beta coronavirus, influenza virus), or therapeutic, for example, activating the subject's immune response to a pathogen after the subject has been exposed to the pathogen (e.g., Beta coronavirus, influenza virus). In some embodiments, an immunogenic composition (e.g., vaccine composition) is used to protect or treat an organism against a disease (e.g., MERS, SARS, COVID-19, and/or influenza).

In some embodiments, the vaccine is a subunit vaccine (e.g., a recombinant subunit Beta coronavirus (e.g., MERS-CoV, SARS-CoV-1, or SARS-CoV-2) or influenza virus (e.g., influenza A virus or influenza B virus) vaccine), an attenuated vaccine (e.g., containing an attenuated Beta coronavirus (e.g., MERS-CoV, SARS-CoV-1, or SARS-CoV-2) or attenuated influenza virus (e.g., influenza A virus or influenza B virus) viral genome), a live vaccine (e.g., containing a live attenuated Beta coronavirus (e.g., MERS-CoV, SARS-CoV-1, or SARS-CoV-2) or a live attenuated influenza virus (e.g., influenza A virus or influenza B virus)), or a conjugated vaccine (e.g., a vaccine containing an antigen (e.g., a Beta coronavirus (e.g., MERS-CoV, SARS-CoV-1, or SARS-CoV-2) antigen, an influenza virus (e.g., influenza A virus or influenza B virus) antigen) that is weakly immunogenic and covalently attached to an antigen that is relatively more immunogenic). One non-limiting example of a conjugated vaccine comprises a LPS attached to a strong protein antigen. In some embodiments, the vaccine comprises a killed/inactivated Beta coronavirus (e.g., MERS-CoV, SARS-CoV-1, or SARS-CoV-2) or a killed/inactivated influenza virus (e.g., influenza A virus or influenza B virus). In some embodiments, the vaccine comprises a Beta coronavirus (e.g., MERS-CoV, SARS-CoV-1, or SARS-CoV-2) or an influenza virus (e.g., influenza A virus or influenza B virus) viral particle.

An “antigen” refers to an entity that is bound by an antibody or receptor, or an entity that induces the production of the antibody. In some embodiments, an antigen increases the production of antibodies that specifically bind the antigen. In some embodiments, an antigen comprises a protein or polypeptide. Such a protein or peptide is referred to herein as an “immunogenic polypeptide.” In some embodiments, the term “antigen” encompasses nucleic acids (e.g., DNA or RNA molecules) that encode immunogenic polypeptides. In some embodiments, the antigen is from a microbial pathogen. For example, the antigen may comprise parts (coats, capsules, cell walls, flagella, fimbriae, and toxins) of bacteria, viruses, fungi, and other microorganisms. As used herein, the term “viral antigen” may refer to an antigen that originates from virus or, in the case, of protein/polypeptide and nucleic acid antigens, has a sequence that is the identical or substantially similar (homologous) to that an endogenous viral protein/polypeptide or nucleic acid. For the purpose of the present disclosure, the antigen may comprise parts of a Beta coronavirus (e.g., MERS-CoV, SARS-CoV-1, or SARS-CoV-2) or an influenza virus (e.g., influenza A virus or influenza B virus).

In some embodiments, a protein or polypeptide antigen is a wild type (i.e., “native”) protein or polypeptide. In some embodiments, a protein or polypeptide antigen is a polypeptide variant to a wild type protein or polypeptide. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, polypeptide variants possess at least 50% identity to a native or reference sequence. In some embodiments, variants share at least 60%, at least 70%, at least 80%, at least 90%, at least 95%, or at least 99% identity with a native or reference sequence.

In some embodiments, a polypeptide variant comprises substitutions, insertions, and/or deletions. In some embodiments, a polypeptide variant encompasses covalent variants and derivatives. The term “derivative” is used synonymously with the term “variant” but generally refers to a molecule that has been modified and/or changed in any way relative to a reference molecule or starting molecule.

In some embodiments, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

In some embodiments, the polypeptide variants comprises at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule. In some embodiments, the antigen is a polypeptide that includes 2, 3, 4, 5, 6, 7, 8, 9, 10, or more substitutions compared to a reference protein.

In some embodiments, the substitution is a conservative amino acids substitution. The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, protein fragments, functional protein domains, and homologous proteins are used as antigens in accordance with the present disclosure. For example, an antigen may comprise any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to a reference protein (e.g., a protein from a microbial pathogen) herein can be utilized in accordance with the disclosure.

In some embodiments, the antigen comprises more than one immunogenic proteins or polypeptides (e.g., 2, 3, 4, 5, 6, 7, 8, 9, 10, or more). In some embodiments, the more than one immunogenic proteins or polypeptides are derived from one protein (e.g., different fragments or one protein). In some embodiments, the more than one immunogenic proteins or polypeptides are derived from multiple proteins (e.g., from 2, 3, 4, 5, 6, 7, 8, 9, 10, or more proteins).

In some embodiments, the antigen comprises a nucleic acid encoding an immunogenic protein or polypeptide. In some embodiments, the antigen comprises an immunogenic protein or polypeptide and a nucleic acid encoding the immunogenic protein or polypeptide. The term “nucleic acid” or “polynucleotide,” in its broadest sense, includes any compound and/or substance that comprises a polymer of nucleotides. Nucleic acids encoding immunogenic proteins or polypeptides typically comprise an open reading frame (ORF), and one or more regulatory sequences. Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, the nucleic acid encoding the immunogenic polypeptide is a DNA (e.g., an expression vector for an immunogenic protein or polypeptide). In some embodiments, the nucleic acid encoding the immunogenic polypeptide is a RNA (e.g., a messenger RNA). A “messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ, or ex vivo. The basic components of an mRNA molecule typically include at least one coding region, a 5′ untranslated region (UTR), a 3′ UTR, a 5′ cap and a poly-A tail.

In some embodiments, the coding region of the nucleic acid (e.g., DNA or RNA) encoding an immunogenic polypeptide is codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding an immunogenic protein or polypeptide). In some embodiments, a codon optimized sequence shares less than 90% sequence identity to a naturally-occurring or wild-type sequence (e.g., a naturally-occurring or wild-type mRNA sequence encoding an immunogenic protein or polypeptide). In some embodiments, a codon optimized sequence shares less than 85% sequence identity to a naturally-occurring or wild-type sequence (e.g., a naturally-occurring or wild-type mRNA sequence encoding an immunogenic protein or polypeptide). In some embodiments, a codon optimized sequence shares less than 80% sequence identity to a naturally-occurring or wild-type sequence (e.g., a naturally-occurring or wild-type mRNA sequence encoding an immunogenic protein or polypeptide). In some embodiments, a codon optimized sequence shares less than 75% sequence identity to a naturally-occurring or wild-type sequence (e.g., a naturally-occurring or wild-type mRNA sequence encoding an immunogenic protein or polypeptide).

In some embodiments, the nucleic acid encoding an immunogenic protein or polypeptide comprises one or more chemical modifications. The terms “chemical modification” and “chemically modified” refer to modification with respect to adenosine (A), guanosine (G), uridine (U), thymidine (T) or cytidine (C) ribonucleosides or deoxyribonucleosides in at least one of their position, pattern, percent or population.

In some embodiments, the nucleic acids (e.g., DNA or RNA) comprise various (more than one) different modifications. In some embodiments, a particular region of a nucleic acid (e.g., DNA or RNA) contains one, two or more (optionally different) nucleoside or nucleotide modifications. In some embodiments, a modified nucleic acid (e.g., DNA or RNA), introduced to a cell or organism, exhibits reduced degradation in the cell or organism, respectively, relative to an unmodified nucleic acid. In some embodiments, a modified nucleic acid (e.g., DNA or RNA), introduced into a cell or organism, may exhibit reduced immunogenicity in the cell or organism, respectively (e.g., a reduced innate response).

Modified nucleic acid (e.g., DNA or RNA) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone). Modified nucleic acid (e.g., DNA or RNA), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

In some embodiments, a chemically modified nucleic acid comprises one or more modified nucleosides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

In some embodiments, a modified nucleobase is a modified uridine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, the antigen comprises a viral protein and/or a nucleic acid encoding a viral protein (e.g., a viral structural or non-structural protein). In some embodiments, the antigen comprises a nucleic acid encoding the viral genome. In some embodiments, the viral genome is modified to produce a modified virus that is attenuated.

Polypeptide or polynucleotide molecules of the present disclosure may share a certain degree of sequence similarity or identity with reference molecules (e.g., reference polypeptides or reference polynucleotides), for example, wild-type molecules. The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g., between nucleic acid molecules (e.g., DNA molecules and/or RNA molecules) and/or between polypeptide molecules. Polymeric molecules (e.g., nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g., DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In some embodiments, the immunogenic compositions (e.g., vaccine compositions) described herein induces an immune response to a Beta coronavirus antigen (e.g., an antigen from any Beta coronavirus such as an antigen from MERS-CoV, SARS-CoV-1, or SARS-CoV-2) or to a Beta coronavirus (any Beta coronavirus species such as MERS-CoV, SARS-CoV-1, or SARS-CoV-2). In some embodiments, Beta coronavirus antigen used in the immunogenic composition described herein comprises an antigen (e.g., a protein or a nucleic acid) from MERS-CoV. In some embodiments, Beta coronavirus antigen used in the immunogenic composition described herein comprises an antigen (e.g., a protein or a nucleic acid) from SARS-CoV-1. In some embodiments, Beta coronavirus antigen used in the immunogenic composition described herein comprises an antigen (e.g., a protein or a nucleic acid) from SARS-CoV-2. In some embodiments, the immunogenic composition (e.g., vaccine composition) induces an immune response against MERS-CoV, SARS-CoV-1 and/or SARS-CoV-2. Heterologous immunity is contemplated herein. Heterologous immunity refers to phenomenon by which antigen-specific response that were generated against one pathogen are reactivated in response to a second pathogen. For example, the immunogenic composition (e.g., vaccine composition) may comprises a SARS-CoV-1 antigen and induces immune response to both SARS-CoV-1 and SARS-CoV-2. Similarly, the immunogenic composition (e.g., vaccine composition) may comprises a SARS-CoV-2 antigen and induces immune response to both SARS-CoV-1 and SARS-CoV-2.

In some embodiments, the Beta coronavirus antigen used in the immunogenic composition (e.g., vaccine composition) described herein comprises a Beta coronavirus (e.g., MERS-CoV, SARS-CoV-1, or SARS-CoV-2) protein or polypeptide, or an immunogenic fragment or variant thereof. In some embodiments, the Beta coronavirus antigen used in the immunogenic composition (e.g., vaccine composition) described herein comprises a nucleic acid (e.g., DNA or RNA such as mRNA) encoding a Beta coronavirus (e.g., MERS-CoV, SARS-CoV-1, or SARS-CoV-2) protein or polypeptide, or an immunogenic fragment or variant thereof.

In some embodiments, the Beta coronavirus antigen in the immunogenic composition (e.g., vaccine composition) described herein comprises a MERS-CoV spike protein, MERS-CoV envelope protein, MERS-CoV membrane protein, MERS-CoV nucleocapsid protein, or an immunogenic fragment thereof (e.g., the receptor binding domain (RBD) of the spike protein). In some embodiments, the Beta coronavirus antigen in the immunogenic composition (e.g., vaccine composition) described herein comprises a nucleic acid (e.g., DNA or RNA such as mRNA) encoding MERS-CoV spike protein, MERS-CoV envelope protein, MERS-CoV membrane protein, MERS-CoV nucleocapsid protein, or an immunogenic fragment thereof (e.g., the receptor binding domain (RBD) of the spike protein).

In some embodiments, the Beta coronavirus antigen in the immunogenic composition (e.g., vaccine composition) described herein comprises a SARS-CoV-1 spike protein, SARS-CoV-1 envelope protein, SARS-CoV-1 membrane protein, SARS-CoV-1 nucleocapsid protein, or an immunogenic fragment thereof (e.g., the receptor binding domain (RBD) of the spike protein). In some embodiments, the Beta coronavirus antigen in the immunogenic composition (e.g., vaccine composition) described herein comprises a nucleic acid (e.g., DNA or RNA such as mRNA) encoding SARS-CoV-1 spike protein, SARS-CoV-1 envelope protein, SARS-CoV-1 membrane protein, SARS-CoV-1 nucleocapsid protein, or an immunogenic fragment thereof (e.g., the receptor binding domain (RBD) of the spike protein).

In some embodiments, the Beta coronavirus antigen in the immunogenic composition (e.g., vaccine composition) described herein comprises a SARS-CoV-2 spike protein, SARS-CoV-2 envelope protein, SARS-CoV-2 membrane protein, SARS-CoV-2 nucleocapsid protein, or an immunogenic fragment thereof (e.g., the receptor binding domain (RBD) of the spike protein). In some embodiments, the Beta coronavirus antigen in the immunogenic composition (e.g., vaccine composition) described herein comprises a nucleic acid (e.g., DNA or RNA such as mRNA) encoding SARS-CoV-2 spike protein, SARS-CoV-2 envelope protein, SARS-CoV-2 membrane protein, SARS-CoV-2 nucleocapsid protein, or an immunogenic fragment thereof (e.g., the receptor binding domain (RBD) of the spike protein).

Amino acid sequences of example Beta coronavirus antigens comprised by the immunogenic composition (e.g., vaccine composition) described herein are provided in Table 1.

TABLE 1
Beta coronavirus protein antigens

| Antigen | Amino Acid Sequence |
|---|---|
| SARS-CoV-1 | MFIFLLFLTLTSGSDLDRCTTFDDV |
| Spike Protein | QAPNYTQHTSSMRGVYYPDEIFRSD |
| (SEQ ID NO: 1) | TLYLTQDLFLPFYSNVTGFHTINHT |
| | FGNPVIPFKDGIYFAATEKSNVVRG |
| | WVFGSTMNNKSQSVIIINNSTNVVI |
| | RACNFELCDNPFFAVSKPMGTQTHT |
| | MIFDNAFNCTFEYISDAFSLDVSEK |
| | SGNFKHLREFVFKNKDGFLYVYKGY |
| | QPIDVVRDLPSGFNTLKPIFKLPLG |
| | INITNFRAILTAFSPAQDIWGTSAA |
| | AYFVGYLKPTTFMLKYDENGTITDA |
| | VDCSQNPLAELKCSVKSFEIDKGIY |
| | QTSNFRVVPSGDVVRFPNITNLCPF |
| | GEVFNATKFPSVYAWERKKISNCVA |
| | DYSVLYNSTFFSTFKCYGVSATKLN |
| | DLCFSNVYADSFVVKGDDVRQIAPG |
| | QTGVIADYNYKLPDDFMGCVLAWNT |
| | RNIDATSTGNYNYKYRYLRHGKLRP |
| | FERDISNVPFSPDGKPCTPPALNCY |
| | WPLNDYGFYTTTGIGYQPYRVVVLS |
| | FELLNAPATVCGPKLSTDLIKNQCV |
| | NFNFNGLTGTGVLTPSSKRFQPFQQ |
| | FGRDVSDFTDSVRDPKTSEILDISP |
| | CSFGGVSVITPGTNASSEVAVLYQD |
| | VNCTDVSTAIHADQLTPAWRIYSTG |
| | NNVFQTQAGCLIGAEHVDTSYECDI |
| | PIGAGICASYHTVSLLRSTSQKSIV |
| | AYTMSLGADSSIAYSNNTIAIPTNF |
| | SISITTEVMPVSMAKTSVDCNMYIC |
| | GDSTECANLLLQYGSFCTQLNRALS |
| | GIAAEQDRNTREVFAQVKQMYKTPT |
| | LKYFGGFNFSQILPDPLKPTKRSFI |
| | EDLLFNKVTLADAGFMKQYGECLGD |
| | INARDLICAQKFNGLTVLPPLLTDD |
| | MIAAYTAALVSGTATAGWTFGAGAA |
| | LQIPFAMQMAYRFNGIGVTQNVLYE |
| | NQKQIANQFNKAISQIQESLTTTST |
| | ALGKLQDVVNQNAQALNTLVKQLSS |
| | NFGAISSVLNDILSRLDKVEAEVQI |
| | DRLITGRLQSLQTYVTQQLIRAAEI |
| | RASANLAATKMSECVLGQSKRVDFC |
| | GKGYHLMSFPQAAPHGVVFLHVTYV |
| | PSQERNFTTAPAICHEGKAYFPREG |
| | VFVFNGTSWFITQRNFFSPQIITTD |
| | NTFVSGNCDVVIGIINNTVYDPLQP |
| | ELDSFKEELDKYFKNHTSPDVDLGD |
| | ISGINASVVNIQKEIDRLNEVAKNL |
| | NESLIDLQELGKYEQYIKWPWYVWL |
| | GFIAGLIAIVMVTILLCCMTSCCSC |
| | LKGACSCGSCCKFDEDDSEPVLKGV |
| | KLHYT |
| SARS-CoV-1 | RVVPSGDVVRFPNITNLCPFGEVFN |
| Spike Protein | ATKFPSVYAWERKKISNCVADYSVL |
| receptor binding | YNSTFFSTFKCYGVSATKLNDLCFS |
| domain (SEQ ID | NVYADSFVVKGDDVRQIAPGQTGVI |
| NO: 2) | ADYNYKLPDDFMGCVLAWNTRNIDA |
| | TSTGNYNYKYR |
| | YLRHGKLRPFERDISNVPFSPDGKP |
| | CTPPALNCYWPLNDYGFYTTTGIGY |
| | QPYRVVVLSFELLNAPATVCGPKLS |
| | TDLIKNQCVNF |
| SARS-CoV-1 | MYSFVSEETGTLIVNSVLLFLAFVV |
| Envelope | FLLVTLAILTALRLCAYCCNIVNVS |
| Protein (SEQ ID | LVKPTVYVYSRVKNLNSSEGVPDLL |
| NO: 3) | V |
| SARS-CoV-1 | MADNGTITVEELKQLLEQWNLVIGF |
| Membrane | LFLAWIMLLQFAYSNRNRFLYIIKL |
| Protein (SEQ ID | VFLWLLWPVTLACFVLAAVYRINWV |
| NO: 4) | TGGIAIAMACIVGLMWLSYFVASFR |
| | LFARTRSMWSFNPETNILLNVPLRG |
| | TIVTRPLMESELVIGAVIIRGHLRM |
| | AGHSLGRCDIKDLPKEITVATSRTL |
| | SYYKLGASQRVGTDSGFAAYNRYRI |
| | GNYKLNTDHAGSNDNIALLVQ |
| SARS-CoV-1 | MSDNGPQSNQRSAPRITFGGPTDST |
| Nucleocapsid | DNNQNGGRNGARPKQRRPQGLPNNT |
| Protein (SEQ ID | ASWFTALTQHGKEELRFPRGQGVPI |
| NO: 5) | NTNSGPDDQIGYYRRATRRVRGGDG |
| | KMKELSPRWYFYYLGTGPEASLPYG |
| | ANKEGIVWVATEGALNTPKDHIGTR |
| | NPNNNAATVLQLPQGTTLPKGFYAE |
| | GSRGGSQASSRSSSRSRGNSRNSTP |
| | GSSRGNSPARMASGGGETALALLLL |
| | DRLNQLESKVSGKGQQQQGQTVTKK |
| | SAAEASKKPRQKRTATKQYNVTQAF |
| | GRRGPEQTQGNFGDQDLIRQGTDYK |
| | HWPQIAQFAPSASAFFGMSRIGMEV |
| | TPSGTWLTYHGAIKLDDKDPQFKDN |
| | VILLNKHIDAYKTFPPTEPKKDKKK |
| | KTDEAQPLPQRQKKQPTVTLLPAAD |
| | MDDFSRQLQNSMSGASADSTQA |
| SARS-CoV-2 | MFVFLVLLPLVSSQCVNLTTRTQLP |
| Spike Protein | PAYTNSFTRGVYYPDKVFRSSVLHS |
| (SEQ ID NO: 6) | TQDLFLPFFSNVTWFHAIHVSGTNG |
| | TKRFDNPVLPFNDGVYFASTEKSNI |
| | IRGWIFGTTLDSKTQSLLIVNNATN |
| | VVIKVCEFQFCNDPFLGVYYHKNNK |
| | SWMESEFRVYSSANNCTFEYVSQPF |
| | LMDLEGKQGNFKNLREFVFKNIDGY |
| | FKIYSKHTPINLVRDLPQGFSALEP |
| | LVDLPIGINITRFQTLLALHRSYLT |
| | PGDSSSGWTAGAAAYYVGYLQPRTF |
| | LLKYNENGTITDAVDCALDPLSETK |
| | CTLKSFTVEKGIYQTSNFRVQPTES |
| | IVRFPNITNLCPFGEVFNATRFASV |
| | YAWNRKRISNCVADYSVLYNSASFS |
| | TFKCYGVSPTKLNDLCFTNVYADSF |
| | VIRGDEVRQIAPGQTGKIADYNYKL |
| | PDDFTGCVIAWNSNNLDSKVGGNYN |
| | YLYRLFRKSNLKPFERDISTEIYQA |
| | GSTPCNGVEGFNCYFPLQSYGFQPT |
| | NGVGYQPYRVVVLSFELLHAPATVC |
| | GPKKSTNLVKNKCVNFNFNGLTGTG |
| | VLTESNKKFLPFQQFGRDIADTTDA |
| | VRDPQTLEILDITPCSFGGVSVITP |
| | GTNTSNQVAVLYQDVNCTEVPVAIH |
| | ADQLTPTWRVYSTGSNVFQTRAGCL |
| | IGAEHVNNSYECDIPIGAGICASYQ |
| | TQTNSPRRARSVASQSIIAYTMSLG |
| | AENSVAYSNNSIAIPTNFTISVTTE |
| | ILPVSMTKTSVDCTMYICGDSTECS |
| | NLLLQYGSFCTQLNRALTGIAVEQD |
| | KNTQEVFAQVKQIYKTPPIKDFGGF |
| | NFSQILPDPSKPSKRSFIEDLLFNK |
| | VTLADAGFIKQYGDCLGDIAARDLI |
| | CAQKFNGLTVLPPLLTDEMIAQYTS |
| | ALLAGTITSGWTFGAGAALQIPFAM |
| | QMAYRFNGIGVTQNVLYENQKLIAN |
| | QFNSAIGKIQDSLSSTASALGKLQD |
| | VVNQNAQALNTLVKQLSSNFGAISS |
| | VLNDILSRLDKVEAEVQIDRLITGR |
| | LQSLQTYVTQQLIRAAEIRASANLA |
| | ATKMSECVLGQSKRVDFCGKGYHLM |
| | SFPQSAPHGVVFLHVTYVPAQEKNF |
| | TTAPAICHDGKAHFPREGVFVSNGT |
| | HWFVTQRNFYEPQIITTDNTFVSGN |
| | CDVVIGIVNNTVYDPLQPELDSFKE |
| | ELDKYFKNHTSPDVDLGDISGINAS |
| | VVNIQ KEIDRLNEVAKNLNESLID |
| | LQELGKYEQYIKWPWYIWLGFIAGL |
| | IAIVMVTIMLCCMTSCCSCLKGCCS |
| | CGSCCKFDEDDSEPVLKGVKLHYT |
| SARS-CoV-2 | RVQPTESIVRFPNITNLCPFGEVFN |
| Spike Protein | ATRFASVYAWNRKRISNCVADYSVL |
| receptor binding | YNSASFSTFKCYGVSPTKLNDLCFT |
| domain (SEQ ID | NVYADSFVIRGDEVRQIAPGQTGKI |
| NO: 7) | ADYNYKLPDDFTGCVIAWNSNNLDS |
| | KVGGNYNYLYRLFRKSNLKPFERDI |
| | STEIYQAGSTPCNGVEGFNCYFPLQ |
| | SYGFQPTNGVGYQPYRVVVLSFELL |
| | HAPATVCGPKKSTNLVKNKCVNF |
| SARS-CoV-2 | MYSFVSEETGTLIVNSVLLFLAFVV |
| Envelope | FLLVTLAILTALRLCAYCCNIVNVS |
| Protein (SEQ ID | LVKPSFYVYSRVKNLNSSRVPDLLV |
| NO: 8) | |
| SARS-CoV-2 | MADSNGTITVEELKKLLEQWNLVIG |
| Membrane | FLFLTWICLLQFAYANRNRFLYIIK |
| Protein (SEQ ID | LIFLWLLWPVTLACFVLAAVYRINW |
| NO: 9) | ITGGIAIAMACLVGLMWLSYFIASF |
| | RLFARTRSMWSFNPETNILLNVPLH |
| | GTILTRPLLESELVIGAVILRGHLR |
| | IAGHHLGRCDIKDLPKEITVATSRT |
| | LSYYKLGASQRVAGDSGFAAYSRYR |
| | IGNYKLNTDHSSSSDNIALLVQ |
| SARS-CoV-2 | MSDNGPQNQRNAPRITFGGPSDSTG |
| Nucleocapsid | SNQNGERSGARSKQRRPQGLPNNTA |
| Protein (SEQ ID | SWFTALTQHGKEDLKFPRGQGVPIN |
| NO: 10) | TNSSPDDQIGYYRRATRRIRGGDGK |
| | MKDLSPRWYFYYLGTGPEAGLPYGA |
| | NKDGIIWVATEGALNTPKDHIGTRN |
| | PANNAAIVLQLPQGTTLPKGFYAEG |
| | SRGGSQASSRSSSRSRNSSRNSTPG |
| | SSRGTSPARMAGNGGDAALALLLLD |
| | RLNQLESKMSGKGQQQQGQTVTKKS |
| | AAEASKKPRQKRTATKAYNVTQAFG |
| | RRGPEQTQGNFGDQELIRQGTDYKH |
| | WPQIAQFAPSASAFFGMSRIGMEVT |
| | PSGTWLTYTGAIKLDDKDPNFKDQV |
| | ILLNKHIDAYKTFPPTEPKKDKKKK |
| | ADETQALPQRQKKQQTVTLLPAADL |
| | DDFSKQLQQSMSSADSTQA |
| MERS-CoV Spike | MIHSVFLLMFLLTPTESYVDVGPDS |
| protein (SEQ ID | VKSACIEVDIQQTFFDKTWPRPIDV |
| NO: 11) | SKADGIIYPQGRTYSNITITYQGLF |
| | PYQGDHGDMYVYSAGHATGTTPQKL |
| | FVANYSQDVKQFANGFVVRIGAAAN |
| | STGTVIISPSTSATIRKIYPAFMLG |
| | SSVGNFSDGKMGRFFNHTLVLLPDG |
| | CGTLLRAFYCILEPRSGNHCPAGNS |
| | YTSFATYHTPATDCSDGNYNRNASL |
| | NSFKEYFNLRNCTFMYTYNITEDEI |
| | LEWFGITQTAQGVHLFSSRYVDLYG |
| | GNMFQFATLPVYDTIKYYSIIPHSI |
| | RSIQSDRKAWAAFYVYKLQPLTFLL |
| | DFSVDGYIRRAIDCGFNDLSQLHCS |
| | YESFDVESGVYSVSSFEAKPSGSVV |
| | EQAEGVECDFSPLLSGTPPQVYNFK |
| | RLVFTNCNYNLTKLLSLFSVNDFTC |
| | SQISPAAIASNCYSSLILDYFSYPL |
| | SMKSDLSVSSAGPISQFNYKQSFSN |
| | PTCLILATVPHNLTTITKPLKYSYI |
| | NKCSRLLSDDRTEVPQLVNANQYSP |
| | CVSTVPSTVWEDGDYYRKQLSPLEG |
| | GGWLVASGSTVAMTEQLQMGFGITV |
| | QYGTDTNSVCPKLEFANDTKIASQL |
| | GNCVEYSLYGVSGRGVFQNCTAVGV |
| | RQQRFVYDAYQNLVGYYSDDGNYYC |
| | LRACVSVPVSVIYDKETKTHATLFG |
| | SVACEHISSTMSQYSRSTRSMLKRR |
| | DSTYGPLQTPVGCVLGLVNSSLFVE |
| | DCKLPLGQSLCALPDTPSTLTPRSV |
| | RSVPGEMRLASIAFNHPIQVDQLNS |
| | SYFKLSIPTNFSFGVTQEYIQTTIQ |
| | KVTVDCKQYVCNGFQKCEQLLREYG |
| | QFCSKINQALHGANLRQDDSVRNLF |
| | ASVKSSQSSPIIPGFGGDFNLTLLE |
| | PVSISTGSRSARSAIEDLLFDKVTI |
| | ADPGYMQGYDDCMQQGPASARDLIC |
| | AQYVAGYKVLPPLMDVNMEAAYTSS |
| | LLGSIAGVGWTAGLSSFAAIPFAQS |
| | IFYRLNGVGITQQVLSENQKLIANK |
| | FNQALGAMQTGFTTTNEAFRKVQDA |
| | VNNNAQALSKLASELSNTFGAISAS |
| | IGDIIQRLDVLEQDAQIDRLINGRL |
| | TTLNAFVAQQLVRSESAALSAQLAK |
| | DKVNECVKAQSKRSGFCGQGTHIVS |
| | FVVNAPNGLYFMHVGYYPSNHIEVV |
| | SAYGLCDAANPTNCIAPVNGYFIKT |
| | NNTRIVDEWSYTGSSFYAPEPITSL |
| | NTKYVAPQVTYQNISTNLPPPLLGN |
| | STGIDFQDELDEFFKNVSTSIPNFG |
| | SLTQINTTLLDLTYEMLSLQQVVKA |
| | LNESYIDLKELGNYTYYNKWPWYIW |
| | LGFIAGLVALALCVFFILCCTGCGT |
| | NCMGKLKCNRCCDRYEEYDLEPHKV |
| | HVH |
| MERS-CoV Spike | ECDFSPLLSGTPPQVYNFKRLVFTN |
| protein receptor | CNYNLTKLLSLFSVNDFTCSQISPA |
| binding domain | AIASNCYSSLILDYFSYPLSMKSDL |
| (SEQ ID NO: 12) | SVSSAGPISQFNYKQSFSNPTCLIL |
| | ATVPHNLTTITKPLKYSYINKC |
| MERS-CoV | MLPFVQERIGLFIVNFFIFTVVCAI |
| Envelope | TLLVCMAFLTATRLCVQCMTGFNTL |
| protein (SEQ ID | LVQPALYLYNTGRSVYVKFQDSKPP |
| NO: 13) | LPPDEWV |
| MERS-CoV | MSNMTQLTEAQIIAIIKDWNFAWSL |
| Membrane | IFLLITIVLQYGYPSRSMTVYVFKM |
| protein (SEQ ID | FVLWLLWPSSMALSIFSAVYPIDLA |
| NO: 14) | SQIISGIVAAVSAMMWISYFVQSIR |
| | LFMRTGSWWSFNPETNCLLNVPFGG |
| | TTVVRPLVEDSTSVTAVVTNGHLKM |
| | AGMHFGACDYDRLPNEVTVAKPNVL |
| | IALKMVKRQSYGTNSGVAIYHRYKA |
| | GNYRSPPITADIELALLRA |
| MERS-CoV | MASPAAPRAVSFADNNDITNTNLSR |
| Nucleocapsid | GRGRNPKPRAAPNNTVSWYTGLTQH |
| protein (SEQ ID | GKVPLTFPPGQGVPLNANSTPAQNA |
| NO: 15) | GYWRRQDRKINTGNGIKQLAPRWYF |
| | YYTGTGPEAALPFRAVKDGIVWVHE |
| | DGATDAPSTFGTRNPNNDSAIVTQF |
| | APGTKLPKNFHIEGTGGNSQSSSRA |
| | SSLSRNSSRSSSQGSRSGNSTRGTS |
| | PGPSGIGAVGGDLLYLDLLNRLQAL |
| | ESGKVKQSQPKVITKKDAAAAKNKM |
| | RHKRTSTKSFNMVQAFGLRGPGDLQ |
| | GNFGDLQLNKLGTEDPRWPQIAELA |
| | PTASAFMGMSQFKLTHQNNDDHGNP |
| | VYFLRYSGAIKLDPKNPNYNKWLEL |
| | LEQNIDAYKTFPKKEKKQKAPKEES |
| | TDQMSEPPKEQRVQGSITQRTRTRP |
| | SVQPGPMIDVNTD |

In some embodiments, the Beta coronavirus antigen in the immunogenic composition (e.g., vaccine composition) described herein comprises a protein having an amino acid sequence that is at least 70% (e.g., at least 70%, at least 75%, at least 80%, at least 85%, at least 90%, at least 95%, or at least 99%) identical to any one of SEQ ID NOs: 1-15. In some embodiments, the Beta coronavirus antigen in the immunogenic composition (e.g., vaccine composition) described herein comprises a protein having an amino acid sequence that is 70%, 75%, 80%, 85%, 90%, 95%, or 99% identical to any one of SEQ ID NOs: 1-15. In some embodiments, the Beta coronavirus antigen in the immunogenic composition (e.g., vaccine composition) described herein comprises a protein comprising the amino acid sequence of any one of SEQ ID NO: 1-15.

In some embodiments, the Beta coronavirus antigen in the immunogenic composition (e.g., vaccine composition) described herein comprises a nucleic acid (e.g., DNA or RNA such as mRNA) encoding a protein having an amino acid sequence that is at least 70% (e.g., at least 70%, at least 75%, at least 80%, at least 85%, at least 90%, at least 95%, or at least 99%) identical to any one of SEQ ID NOs: 1-15. In some embodiments, the Beta coronavirus antigen in the immunogenic composition (e.g., vaccine composition) described herein comprises a nucleic acid (e.g., DNA or RNA such as mRNA) encoding a protein having an amino acid sequence that is 70%, 75%, 80%, 85%, 90%, 95%, or 99% identical to any one of SEQ ID NOs: 1-15. In some embodiments, the Beta coronavirus antigen in the immunogenic composition (e.g., vaccine composition) described herein comprises a nucleic acid (e.g., DNA or RNA such as mRNA) encoding a protein comprising the amino acid sequence of any one of SEQ ID NO: 1-15.

In some embodiments, the immunogenic compositions (e.g., vaccine compositions) described herein induces an immune response to an influenza virus antigen (e.g., an antigen from any influenza virus such as an antigen from an influenza A virus or an influenza B virus) or to an influenza A virus or an influenza B virus (i.e., any strain of influenza A virus or influenza B virus). In some embodiments, the influenza virus antigen used in the immunogenic composition described herein comprises an antigen (e.g., a protein or a nucleic acid) from an influenza A virus. In some embodiments, the influenza virus antigen used in the immunogenic composition described herein comprises an antigen (e.g., a protein or a nucleic acid) from an influenza B virus. In some embodiments, the immunogenic composition (e.g., vaccine composition) induces an immune response against an influenza A virus or an influenza B virus. Heterologous immunity is contemplated herein. Heterologous immunity refers to phenomenon by which antigen-specific response that were generated against one pathogen are reactivated in response to a second pathogen. For example, the immunogenic composition (e.g., vaccine composition) may comprises an influenza A virus antigen and induces immune response to both an influenza A virus and an influenza B virus. Similarly, the immunogenic composition (e.g., vaccine composition) may comprises an influenza B virus antigen and induces immune response to both an influenza B virus and an influenza A virus. Similarly, the immunogenic composition (e.g., vaccine composition) may induce a immune response to multiple subtypes (strains) of influenza A virus or to multiple subtypes (strains) of influenza B virus.

In some embodiments, the influenza virus antigen used in the immunogenic composition (e.g., vaccine composition) described herein comprises an influenza virus (e.g., influenza A virus, influenza B virus) protein or polypeptide, or an immunogenic fragment or variant thereof. In some embodiments, the influenza virus antigen used in the immunogenic composition (e.g., vaccine composition) described herein comprises a nucleic acid (e.g., DNA or RNA such as mRNA) encoding an influenza virus (e.g., influenza A virus, influenza B virus) protein or polypeptide, or an immunogenic fragment or variant thereof.

In some embodiments, the influenza virus antigen in the immunogenic composition (e.g., vaccine composition) described herein comprises an influenza A virus hemagglutinin (HA) protein, an influenza A virus neuraminidase (NA) protein, or an immunogenic fragment thereof. In some embodiments, the influenza A virus antigen in the immunogenic composition (e.g., vaccine composition) described herein comprises a nucleic acid (e.g., DNA or RNA such as mRNA) encoding an influenza A virus hemagglutinin (HA) protein, an influenza A virus neuraminidase (NA) protein, or an immunogenic fragment thereof.

In some embodiments, the influenza virus antigen in the immunogenic composition (e.g., vaccine composition) described herein comprises an influenza B virus hemagglutinin (HA) protein, an influenza B virus neuraminidase (NA) protein, or an immunogenic fragment thereof. In some embodiments, the influenza A virus antigen in the immunogenic composition (e.g., vaccine composition) described herein comprises a nucleic acid (e.g., DNA or RNA such as mRNA) encoding an influenza B virus hemagglutinin (HA) protein, an influenza B virus neuraminidase (NA) protein, or an immunogenic fragment thereof.

Amino acid sequences of example influenza virus antigens comprised by the immunogenic composition (e.g., vaccine composition) described herein are provided in Table 2.

TABLE 2
Influenza virus protein antigens

| Antigen | Amino Acid Sequence |
|---|---|
| Influenza A virus | MKAILVVLLYTFATANADTLCIGYH |
| hemagglutinin | ANNSTDTVDTVLEKNVTVTHSVNLL |
| (A/Jakarta/009/ | EDKHNGKLCKLRGVAPLHLGKCNIA |
| 2009 (H1N1)) | GWILGNPECESLSTASSWSYIVETS |
| (SEQ ID NO: 16) | SSDNGTCYPGDFIDYEELREQLSSV |
| | SSFERFEIFPKTSSWPNHDSNKGVT |
| | AACPHAGAKSFYKNLIWLVKKGNSY |
| | PKLSKSYINDKGKEVLVLWGIHHPS |
| | TSADQQSLYQTADAYVFVGTSRYSK |
| | KFKPEIAIRPKVRDQEGRMNYYWTL |
| | VEPGDKITFEATGNLVVPRYAFAME |
| | RNAGSGIIISDTPVHDCNTTCQTPK |
| | GAINTSLPFQNIHPITIGKCPKYVK |
| | STKLRLATGLRNVPSIQSRGLFGAI |
| | AGFIEGGWTGMVDGWYGYHHQNEQG |
| | SGYAADLKSTQNAIDEITNKVNSVI |
| | EKMNTQFTAVGKEFNHLEKRIENLN |
| | KKVDDGFLDIWTYNAELLVLLENER |
| | TLDYHDSNVKNLYEKVRSQLKNNAK |
| | EIGNGCFEFYHKCDNTCMESVKNGT |
| | YDYPKYSEEAKLNREEIDGVKLEST |
| | RIYQILAIYSTVASSLVLVVSLGAI |
| | SFWMCSNGSLQCRICI |
| Influenza A virus | MNPNQKIITIGSVCMTIGMANLILQ |
| neuraminidase | IGNIISIWISHSIQLGNQNQIETCN |
| (A/California/07 | QSVITYENNTWVNQTYVNISNTNFA |
| /2009 (H1N1)) | AGQSVVSVKLAGNSSLCPVSGWAIY |
| (SEQ ID NO: 17) | SKDNSVRIGSKGDVFVIREPFISCS |
| | PLECRTFFLTQGALLNDKHSNGTIK |
| | DRSPYRTLMSCPIGEVPSPYNSRFE |
| | SVAWSASACHDGINWLTIGISGPDN |
| | GAVAVLKYNGIITDTIKSWRNNILR |
| | TQESECACVNGSCFTVMTDGPSNGQ |
| | ASYKIFRIEKGKIVKSVEMNAPNYH |
| | YEECSCYPDSSEITCVCRDNWHGSN |
| | RPWVSFNQNLEYQIGYICSGIFGDN |
| | PRPNDKTGSCGPVSSNGANGVKGFS |
| | FKYGNGVWIGRTKSISSRNGFEMIW |
| | DPNGWTGTDNNFSIKQDIVGINEWS |
| | GYSGSFVQHPELTGLDCIRPCFWVE |
| | LIRGRPKENTIWTSGSSISFCGVNS |
| | DTVGWSWPDGAELPFTIDK |
| Influenza B virus | MKAIIVLLMVVTSNADRICTGITSS |
| hemagglutinin | NSPHVVKTATQGEVNVTGVIPLTTT |
| (B/Lee/1940) | PTKSHFANLKGTQTRGKLCPNCFNC |
| (SEQ ID NO: 18) | TDLDVALGRPKCMGNTPSAKVSILH |
| | EVKPATSGCFPIMHDRTKIRQLPNL |
| | LRGYENIRLSTSNVINTETAPGGPY |
| | KVGTSGSCPNVANGNGFFNTMAWVI |
| | PKDNNKTAINPVTVEVPYICSEGED |
| | QITVWGFHSDDKTQMERLYGDSNPQ |
| | KFTSSANGVTTHYVSQIGGFPNQTE |
| | DEGLKQSGRIVVDYMVQKPGKTGTI |
| | VYQRGILLPQKVWCASGRSKVIKGS |
| | LPLIGEADCLHEKYGGLNKSKPYYT |
| | GEHAKAIGNCPIWVKTPLKLANGTK |
| | YRPPAKLLKERGFFGAIAGFLEGGW |
| | EGMIAGWHGYTSHGAHGVAVAADLK |
| | STQEAINKI |
| | TKNLNYLSELEVKNLQRLSGAMNEL |
| | HDEILELDEKVDDLRADTISSQIEL |
| | AVLLSNEGIINSEDEHLLALERKLK |
| | KMLGPSAVEIGNGCFETKHKCNQTC |
| | LDRIAAGTFNAGDFSLPTFDSLNIT |
| | AASLNDDGLDNHTILLYYSTAASSL |
| | AVTLMIAIFIVYMVSRDNVSCSICL |
| Influenza B virus | MLPSTVQTLTLLLTSGGVLLSLYVS |
| neuraminidase | ASLSYLLYSDVLLKFSSTKTTAPTM |
| (B/Lee/1940) | SLECTNASNAQTVNHSATKEMTFPP |
| (SEQ ID NO: 19) | PEPEWTYPRLSCQGSTFQKALLISP |
| | HRFGEIKGNSAPLIIREPFVACGPK |
| | ECRHFALTHYAAQPGGYYNGTRKDR |
| | NKLRHLVSVKLGKIPTVENSIFHMA |
| | AWSGSACHDGREWTYIGVDGPDNDA |
| | LVKIKYGEAYTDTYHSYAHNILRTQ |
| | ESACNCIGGDCYLMITDGSASGISK |
| | CRFLKIREGRIIKEILPTGRVEHTE |
| | ECTCGFASNKTIECACRDNSYTAKR |
| | PFVKLNVETDTAEIRLMCTKTYLDT |
| | PRPDDGSIAGPCESNGDKWLGGIKG |
| | GFVHQRMASKIGRWYSRTMSKTNRM |
| | GMELYVKYDGDPWTDSDALTLSGVM |
| | VSIEEPGWYSFGFEIKDKKCDVPCI |
| | GIEMVHDGGKDTWHSAATAIYCLMG |
| | SGQLLWDTVTGVDMAL |

In some embodiments, the influenza virus antigen in the immunogenic composition (e.g., vaccine composition) described herein comprises a protein having an amino acid sequence that is at least 70% (e.g., at least 70%, at least 75%, at least 80%, at least 85%, at least 90%, at least 95%, or at least 99%) identical to any one of SEQ ID NOs: 16-19, or to the amino acid sequence of another influenza virus antigen known in the art (e.g., an influenza virus antigen of another subtype). In some embodiments, the influenza virus antigen in the immunogenic composition (e.g., vaccine composition) described herein comprises a protein having an amino acid sequence that is 70%, 75%, 80%, 85%, 90%, 95%, or 99% identical to any one of SEQ ID NOs: 16-19, or to the amino acid sequence of another influenza virus antigen known in the art (e.g., an influenza virus antigen of another subtype). In some embodiments, the influenza virus antigen in the immunogenic composition (e.g., vaccine composition) described herein comprises a protein comprising the amino acid sequence of any one of SEQ ID NO: 16-19, or the amino acid sequence of another influenza virus antigen known in the art (e.g., an influenza virus antigen of another subtype).

In some embodiments, the influenza virus antigen in the immunogenic composition (e.g., vaccine composition) described herein comprises a nucleic acid (e.g., DNA or RNA such as mRNA) encoding a protein having an amino acid sequence that is at least 70% (e.g., at least 70%, at least 75%, at least 80%, at least 85%, at least 90%, at least 95%, or at least 99%) identical to any one of SEQ ID NOs: 16-19, or to the amino acid sequence of another influenza virus antigen known in the art (e.g., an influenza virus antigen of another subtype). In some embodiments, the influenza virus antigen in the immunogenic composition (e.g., vaccine composition) described herein comprises a nucleic acid (e.g., DNA or RNA such as mRNA) encoding a protein having an amino acid sequence that is 70%, 75%, 80%, 85%, 90%, 95%, or 99% identical to any one of SEQ ID NOs: 16-19, or to the amino acid sequence of another influenza virus antigen known in the art (e.g., an influenza virus antigen of another subtype). In some embodiments, the influenza virus antigen in the immunogenic composition (e.g., vaccine composition) described herein comprises a nucleic acid (e.g., DNA or RNA such as mRNA) encoding a protein comprising the amino acid sequence of any one of SEQ ID NO: 16-19, or the amino acid sequence of another influenza virus antigen known in the art (e.g., an influenza virus antigen of another subtype).

In some embodiments, the immunogenic composition (e.g., vaccine composition) described herein are formulated for administration to a subject. In some embodiments, the immunogenic composition (e.g., vaccine composition) is formulated or administered in combination with one or more pharmaceutically-acceptable excipients. In some embodiments, immunogenic compositions (e.g., vaccine composition) comprise at least one additional active substances, such as, for example, a therapeutically-active substance, a prophylactically-active substance, or a combination of both. Immunogenic compositions (e.g., vaccine composition) may be sterile, pyrogen-free or both sterile and pyrogen-free. General considerations in the formulation and/or manufacture of pharmaceutical agents, such as immunogenic compositions (e.g., vaccine composition), may be found, for example, in Remington: The Science and Practice of Pharmacy 21st ed., Lippincott Williams & Wilkins, 2005 (incorporated herein by reference in its entirety).

Formulations of the immunogenic composition (e.g., vaccine composition) described herein may be prepared by any method known or hereafter developed in the art of pharmacology. In general, such preparatory methods include the step of bringing the antigen and/or the adjuvant (e.g., fungal polysaccharide or fungal polysaccharide and alum) into association with an excipient and/or one or more other accessory ingredients, and then, if necessary and/or desirable, dividing, shaping and/or packaging the product into a desired single- or multi-dose unit.

Relative amounts of the antigen, the adjuvant, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the immunogenic composition (e.g., vaccine composition) described herein are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA vaccines (e.g., for transplantation into a subject), hyaluronidase, nanoparticle mimics and combinations thereof.

In some embodiments, the immunogenic composition (e.g., vaccine composition) is formulated in an aqueous solution. In some embodiments, the immunogenic composition (e.g., vaccine composition) is formulated in a nanoparticle. In some embodiments, the immunogenic composition (e.g., vaccine composition) is formulated in a lipid nanoparticle. In some embodiments, the immunogenic composition (e.g., vaccine composition) is formulated in a lipid-polycation complex, referred to as a lipid nanoparticle. The formation of the lipid nanoparticle may be accomplished by methods known in the art and/or as described in U.S. Pub. No. 20120178702, incorporated herein by reference. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is incorporated herein by reference. In some embodiments, the immunogenic composition (e.g., vaccine composition) is formulated in a lipid nanoparticle that includes a non-cationic lipid such as, but not limited to, cholesterol or dioleoyl phosphatidylethanolamine (DOPE).

In some embodiments, a vaccine formulation described herein is a nanoparticle that comprises at least one lipid (termed a “lipid nanoparticle” or “LNP”). The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, incorporated herein by reference. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, the immunogenic composition (e.g., vaccine composition) described herein may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm. In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the immunogenic composition (e.g., vaccine composition) is formulated in a liposome. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are incorporated herein by reference.

In some embodiments, the immunogenic composition (e.g., vaccine composition) described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; incorporated herein by reference) and liposomes which may deliver small molecule drugs such as, but not limited to, DOXIL® from Janssen Biotech, Inc. (Horsham, PA).

In some embodiments, the antigen and/or the adjuvantation system may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are incorporated herein by reference.

The antigen, the adjuvantation system, and/or optionally the second adjuvant may be formulated using any of the methods described herein or known in the art separately or together. For example, the antigen and the adjuvantation system may be formulated in one lipid nanoparticle or two separately lipid nanoparticles. In some embodiments, the antigen, the adjuvantation system are formulated in the same aqueous solution or two separate aqueous solutions.

Other aspects of the present disclosure provide methods of inducing an immune response to a virus or a viral antigen in a subject in need thereof, the method comprising administering to the subject an effective amount of a virus or viral antigen and an effective amount of an adjuvantation system comprising a fungal polysaccharide (e.g., a mannan). In some embodiments, the present disclosure provides methods of inducing an immune response to Beta coronavirus (e.g., MERS-CoV, SARS-CoV-1, or SARS-CoV-2) or influenza virus (e.g., influenza A virus or influenza B virus), or a Beta coronavirus (e.g., MERS-CoV, SARS-CoV-1, or SARS-CoV-2) or influenza virus (e.g., influenza A virus or influenza B virus) antigen in a subject in need thereof, the method comprising administering to the subject an effective amount of a Beta coronavirus (e.g., MERS-CoV, SARS-CoV-1, or SARS-CoV-2) or influenza virus (e.g., influenza A virus or influenza B virus) antigen and an effective amount of an adjuvantation system comprising a fungal polysaccharide (e.g., a mannan). In some embodiments, the adjuvantation system further comprises alum. In some embodiments, the fungal polysaccharide (e.g., a mannan) is adsorbed into the alum.

In some embodiments, the adjuvantation system (e.g., comprising a fungal polysaccharide such as mannan alone or a fungal polysaccharide and alum) is administered separately from the viral antigen. In some embodiments, the adjuvantation system (e.g., comprising a fungal polysaccharide such as mannan alone or a fungal polysaccharide and alum) is administered prior to administering the viral antigen. In some embodiments, the adjuvantation system (e.g., comprising a fungal polysaccharide such as mannan alone or a fungal polysaccharide and alum) is administered after administering the viral antigen. In some embodiments, the adjuvantation system (e.g., comprising a fungal polysaccharide such as mannan alone or a fungal polysaccharide and alum) and the viral antigen are administered simultaneously. In some embodiments, the adjuvantation system (e.g., comprising a fungal polysaccharide such as mannan alone or a fungal polysaccharide and alum) and the viral antigen are administered as an admixture.

A “subject” to which administration is contemplated refers to a human (i.e., male or female of any age group, e.g., pediatric subject (e.g., infant, child, or adolescent) or adult subject (e.g., young adult, middle-aged adult, or senior adult)), or a non-human animal. In some embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In some embodiments, the non-human animal is a fish, reptile, or amphibian. The non-human animal may be a male or female at any stage of development. The non-human animal may be a transgenic animal or a genetically engineered animal. A “subject in need thereof” refers to a subject (e.g., a human subject or a non-human mammal) in need of treatment of infection by a virus, such as a Beta coronavirus (e.g., a subject having MERS, SARS or COVID-19) or an influenza virus (e.g., a subject having influenza), or in need of reducing the risk of developing an infection by a virus, such as Beta coronavirus (e.g., MERS-CoV, SARS-CoV-1, or SARS-CoV-2) or an influenza virus (e.g., an influenza A virus or an influenza B virus). In some embodiments, administering the viral antigen, such as a Beta coronavirus (e.g., MERS-CoV, SARS-CoV-1, or SARS-CoV-2) or influenza virus (e.g., an influenza A virus or an influenza B virus) antigen, and the adjuvantation system described herein (e.g., comprising a fungal polysaccharide such as mannan alone or a fungal polysaccharide and alum) to a subject having an infection caused by the virus, such as a Beta coronavirus (e.g., MERS-CoV, SARS-CoV-1, or SARS-CoV-2) or influenza virus (e.g., an influenza A virus or an influenza B virus) infection, treats (i.e., has a therapeutic use for) the disease (MERS, SARS, COVID-19, or influenza). In some embodiments, administering the antigen and the adjuvantation system described herein (e.g., comprising a fungal polysaccharide such as mannan alone or a fungal polysaccharide and alum) to a subject at risk of developing an infection by a virus, such as a Beta coronavirus (e.g., MERS-CoV, SARS-CoV-1, or SARS-CoV-2) or an influenza virus (e.g., an influenza A virus or an influenza B virus), reduces the likelihood (e.g., by 20%, 30%, 40%, 50%, 60%, 70%, 80%, 90%, 95%, 99% or more) of the subject developing the infection (prophylactic use).

In some embodiments, the subject is a human subject, e.g., a human neonate, infant, child, adult, or elderly. In some embodiments, the fungal polysaccharide used in the adjuvantation system for enhancing an immune response to a virus, such as a Beta coronavirus (e.g., MERS-CoV, SARS-CoV-1, or SARS-CoV-2) or influenza virus (e.g., an influenza A virus or an influenza B virus), in a human subject is mannan (e.g., mannan alone or mannan formulated with alum). In some embodiments, the fungal polysaccharide used in the adjuvantation system for enhancing an immune response to a virus, such as a Beta coronavirus (e.g., MERS-CoV, SARS-CoV-1, or SARS-CoV-2) or influenza virus (e.g., an influenza A virus or an influenza B virus), in a human subject is β-glucan. (e.g., β-glucan alone or β-glucan formulated with alum). In some embodiments, the fungal polysaccharide used in the adjuvantation system for enhancing an immune response to a virus, such as a Beta coronavirus (e.g., MERS-CoV, SARS-CoV-1, or SARS-CoV-2) or influenza virus (e.g., an influenza A virus or an influenza B virus), in a human subject is fungal polysaccharide (e.g., alone or formulated with alum) isolated from Candida albicans.

In some embodiments, a human subject to which the contemplated adjuvantation system for enhancing an immune response to the virus (e.g., Beta coronavirus or influenza virus) is administered is a newborn or more than 1 month, 2 months, 3 months, 4 months, 5 months, 6 months, 7 months, 8 months, 9 months, 10 months, 11 months, 1 year, 2 years, 3 years, 4 years, 5 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years old. In some embodiments, the human subject is an adult human (e.g., more than 18 years old). In some embodiments, the human subject is an elderly human (e.g., more than 60 years old). In some embodiments, the human subject is more than 65 years of age. In some embodiments, the human subject receives (i.e., is administered) one or more doses of the vaccine described herein.

In some embodiments, the human subject has an undeveloped (e.g., an infant or a neonate), weak (an elderly), or compromised immune system. Immunocompromised subjects include, without limitation, subjects with primary immunodeficiency or acquired immunodeficiency such as those suffering from sepsis, HIV infection, and cancers, including those undergoing chemotherapy and/or radiotherapy. In some embodiments, the human subject has an underlying condition that renders them more susceptible to an infection caused by a virus, such as a Beta coronavirus (e.g., MERS-CoV, SARS-CoV-1, or SARS-CoV-2) and/or influenza virus (e.g., an influenza A virus or an influenza B virus) infection. In some embodiments, the human subject is immunocompromised, has chronic lung disease, asthma, cardiovascular disease, cancer, obesity, diabetes, chronic kidney disease, and/or liver disease.

In some embodiments, the subject is a companion animal (i.e., a pet or service animal). The use of the immunogenic composition (e.g., vaccine composition) described herein in a veterinary vaccine is also within the scope of the present disclosure. “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., ferrets, pigs, rats, mice, guinea pigs, and hamsters), rabbits, or non-human primates.

Once administered, the immunogenic composition (e.g., vaccine composition) described herein elicits an immune response in the subject. In some embodiments, the immune response is type 1 immune response, characterized by production and secretion of IgE antibodies from B cells, vasodilation, and leukocyte extravasation. In some embodiments, the immune response is an innate immune response. In some embodiments, the immune response is an adaptive immune response specific to the antigen in the composition or vaccine. In some embodiments, the immunogenic composition (e.g., vaccine composition) described herein activates B cell immunity. In some embodiments, the immunogenic composition (e.g., vaccine composition) elicits production of antibodies (immunoglobulins, e.g., IgE, IgG, IgA, IgM, or sub-types thereof, e.g., IgG1, IgG2, IgG3, and IgG4) against the antigen. In some embodiments, the immunogenic composition (e.g., vaccine composition) activates cytotoxic T cells specific to the antigen. In some embodiments, the immunogenic composition (e.g., vaccine composition) elicits production of cytokines (e.g., chemokines, interferons, interleukins, etc.) by antigen-specific T cells.

In some embodiments, the fungal polysaccharide used in the adjuvantation system for enhancing an immune response to a virus, such as a Beta coronavirus (e.g., MERS-CoV, SARS-CoV-1, or SARS-CoV-2) or influenza virus (e.g., an influenza A virus or an influenza B virus), in a human subject elicits an innate immune response by activating a pattern recognition receptor (PRR) on immune cells of the subject. PRRs are critical to innate immunity and act by detecting the presence of particular microbial antigens and in response signaling for production and secretion of inflammatory cytokines. In some embodiments, the PRR is a lectin (i.e., a receptor that binds to specific carbohydrates), such as a C-type lectin receptor (CLR). In some embodiments, the PRR is dendritic cell-associated C-type lectin 1 (Dectin-1). In some embodiments, the PRR is dendritic cell-associated C-type lectin 2 (Dectin-2).

In some embodiments, the adjuvantation system described herein (e.g., fungal polysaccharide alone, or fungal polysaccharide formulated with alum), whether administered alone or in an admixture with a viral antigen, leads to the production of antibodies (immunoglobulins, e.g., IgE, IgG, IgA, IgM, or sub-types thereof, e.g., IgG1, IgG2, IgG3, and IgG4) targeting a wider (i.e. broader) range of epitopes of the antigen, compared to without the adjuvantation system or when the viral antigen is administered alone. An “epitope” is defined as an amino acid sequence of an antigen that is specifically targeted by an antibody, compared to other amino acid sequences of the antigen. In some embodiments, for example, the range of epitopes for which antigen-specific antibodies are produced may be increased by 1, increased by 2, increased by 3, increased by 4, increased by 5, increased by 6, increased by 7, increased by 8, increased by 9, increased by 10, or increased by more than 10 in the presence of the adjuvantation system, compared to without the adjuvantation system or when the viral antigen is administered alone

In some embodiments, the adjuvantation system described herein (e.g., fungal polysaccharide alone, or fungal polysaccharide formulated with alum) enhances the production of a proinflammatory cytokine (e.g., IFNγ+) in the subject. In some embodiments, the level of proinflammatory cytokines (e.g., IFNγ+) is increased by at least 20% in the presence of the adjuvantation system, compared to without the adjuvantation system or when the viral antigen is administered alone. For example, the level of proinflammatory cytokines (e.g., IFNγ+) may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the adjuvantation system, compared to without the adjuvantation system or when the viral antigen is administered alone. In some embodiments, the level of proinflammatory cytokines (e.g., IFNγ+) is increased by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the viral antigen is administered alone.

In some embodiments, the adjuvantation system described herein (e.g., fungal polysaccharide alone, or fungal polysaccharide formulated with alum), whether administered alone or in an admixture with a viral antigen, enhance the innate immune response, compared to without the adjuvantation system or when the viral antigen is administered alone. In some embodiments, the adjuvantation system described herein (e.g., fungal polysaccharide alone, or fungal polysaccharide formulated with alum) activates newborn or elderly peripheral blood mononuclear cells (PBMCs). In some embodiments, the number of PBMCs that are activated is increased by at least 20% in the presence of the adjuvantation system described herein (e.g., e.g., fungal polysaccharide alone, or fungal polysaccharide formulated with alum), compared to without the adjuvantation system or when the viral antigen is administered alone. For example, the number of PBMCs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the adjuvantation system, compared to without the adjuvantation system or when the viral antigen is administered alone. In some embodiments, the number of PBMCs that are activated is increased by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the viral antigen is administered alone.

In some embodiments, the adjuvantation system enhances innate immune memory (also referred to as trained immunity). “Innate immune memory” confers heterologous immunity that provides broad protection against a range of pathogens. In some embodiments, the innate immune memory is increased by at least 20% in the presence of the adjuvantation system, compared to without the adjuvantation system or when the viral antigen is administered alone. For example, the innate immune memory may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the adjuvantation system, compared to without the adjuvantation system or when the viral antigen is administered alone. In some embodiments, the innate immune memory is increased by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the viral antigen is administered alone.

In some embodiments, the adjuvantation system, when administered as an admixture with a viral antigen, enhances the anti-specific immune response against the viral antigen, such as a Beta coronavirus (e.g., MERS-CoV, SARS-CoV-1, or SARS-CoV-2) or influenza virus (e.g., an influenza A virus or an influenza B virus) antigen, or against the virus, such as a Beta coronavirus (e.g., MERS-CoV, SARS-CoV-1, or SARS-CoV-2) or influenza virus (e.g., an influenza A virus or an influenza B virus), compared to without the adjuvantation system or when the viral antigen is administered alone. In some embodiments, the adjuvantation system enhances the production of antigen-specific antibody titer (e.g., by at least 20%) in the subject, compared to without the adjuvantation system or when the viral antigen is administered alone. For example, the adjuvantation system may enhance the production of antigen-specific antibody titer by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. in the subject, compared to without the adjuvantation system or when the viral antigen is administered alone. In some embodiments, the adjuvantation system enhances the production of antigen-specific antibody titer by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the viral antigen is administered alone. One skilled in the art is familiar with how to evaluate the level of an antibody titer, e.g., by ELISA.

In some embodiments, the adjuvantation system polarizes the innate and adaptive immune response by shaping the pattern of cytokine and/or chemokine responses toward T helper 1 (Th1) immunity, important for host defense against intracellular pathogens. In some embodiments, the adjuvantation system polarizes the innate immune response toward T follicular helper (Tfh) cell immunity.

In some embodiments, the adjuvantation system prolongs the effect of a vaccine (e.g., by at least 20%) in the subject, compared to without the adjuvantation system or when the viral antigen is administered alone. For example, the adjuvantation system may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more. in the subject, compared to without the adjuvantation system or when the viral antigen is administered alone. In some embodiments, the adjuvantation system prolongs the effect of a vaccine by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the viral antigen is administered alone.

In some embodiments, the adjuvantation system increases rate of (accelerates) an immune response, compared to without the adjuvantation system or when the viral antigen is administered alone. For example, the adjuvantation system may increase the rate of an immune response by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject, compared to without the adjuvantation system or when the viral antigen is administered alone. In some embodiments, the adjuvantation system increases the rate of an immune response by 20%, 30%, 40%, 50%, 60%, 70%, 80%, 90%, 100%, 2-fold, 5-fold, 10-fold, 100-fold, 1000-fold or more, in the presence of the adjuvantation system, compared to without the adjuvantation system or when the viral antigen is administered alone. To “increase the rate of immune response” means less time is required for the immune system of a subject to react to an invading virus, such as a Beta coronavirus (e.g., MERS-CoV, SARS-CoV-1, or SARS-CoV-2) or an influenza virus (e.g., an influenza A virus or an influenza B virus), or viral antigen, such as a Beta coronavirus antigen or an influenza virus antigen.

In some embodiments, the antigen produces the same level of immune response against the viral antigen, such as a Beta coronavirus (e.g., MERS-CoV, SARS-CoV-1, or SARS-CoV-2) or influenza virus (e.g., an influenza A virus or an influenza B virus) antigen, at a lower dose in the presence of the adjuvantation system, compared to without the adjuvantation system or when the viral antigen is administered alone. In some embodiments, the amount of viral antigen needed to produce the same level of immune response is reduced by at least 20% in the presence of the adjuvantation system, compared to without the adjuvantation system or when the viral antigen is administered alone. For example, the amount of antigen needed to produce the same level of immune response may be reduced by at least 20%, at least 30%, at least 40%, at least 50%, at least 60%, at least 70%, at least 80%, at least 90%, at least 95%, at least 99% or more, in the presence of the adjuvantation system, compared to without the adjuvantation system or when the viral antigen is administered alone. In some embodiments, the amount of antigen needed to produce the same level of immune response is reduced by at 20%, 30%, 40%, 50%, 60%, 70%, 80%, 90%, 95%, 99% or more, in the presence of the adjuvantation system, compared to without the adjuvantation system or when the viral antigen is administered alone.

The prophylactic or therapeutic use of the adjuvantation system, or the immunogenic composition (e.g., vaccine composition) described herein is also within the scope of the present disclosure. In some embodiments, the composition or immunogenic composition (e.g., vaccine composition) described herein are used in methods of vaccinating a subject by prophylactically administering to the subject an effective amount of the composition or immunogenic composition (e.g., vaccine composition) described herein. “Vaccinating a subject” refers to a process of administering an immunogen, typically an antigen formulated into a vaccine, to the subject in an amount effective to increase or activate an immune response against the viral antigen, such as a Beta coronavirus antigen (e.g., MERS-COV, SARS-COV-1, SARS-COV-2) or influenza virus (e.g., an influenza A virus or an influenza B virus) antigen, and thus against the virus (e.g., Beta coronavirus (e.g., MERS-COV, SARS-COV-1, SARS-COV-2) or influenza virus (e.g., an influenza A virus or an influenza B virus)). In some embodiments, the term “vaccinating a subject” does not require the creation of complete immunity against the virus. In some embodiments, the term “vaccinating a subject” encompasses a clinically favorable enhancement of an immune response toward the viral antigen or pathogen. Methods for immunization, including formulation of an immunogenic composition (e.g., vaccine composition) and selection of doses, routes of administration and the schedule of administration (e.g., primary dose and one or more booster doses), are well known in the art. In some embodiments, vaccinating a subject reduces the risk of developing a viral infection, such as a Beta coronavirus (e.g., MERS-CoV, SARS-CoV-1, or SARS-CoV-2) or influenza virus (e.g., an influenza A virus or an influenza B virus) infection, and diseases that occur as a result of viral infection, such as those caused by Beta coronavirus infection (e.g., MERS, SARS and/or COVID-19) or influenza virus infection (e.g., influenza).

In some embodiments, the immunogenic compositions (e.g., vaccine composition) described herein are formulated for administration to a subject. In some embodiments, the composition or immunogenic composition (e.g., vaccine composition) further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the composition or immunogenic composition (e.g., vaccine composition) described herein also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The immunogenic composition (e.g., vaccine composition) described herein may conveniently be presented in unit dosage form and may be prepared by any of the methods well-known in the art of pharmacy. The term “unit dose” when used in reference to a composition or immunogenic composition (e.g., vaccine composition) described herein of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the composition or immunogenic compositions (e.g., vaccine composition) described herein may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the composition or immunogenic composition (e.g., vaccine composition) described herein can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the immunogenic composition (e.g., vaccine composition) described herein used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the composition or immunogenic composition (e.g., vaccine composition) described herein ordinarily will be stored in lyophilized form or as an aqueous solution if it is highly stable to thermal and oxidative denaturation. The pH of the preparations typically will be about from 6 to 8, although higher or lower pH values can also be appropriate in certain instances. The chimeric constructs of the present disclosure can be used as vaccines by conjugating to soluble immunogenic carrier molecules. Suitable carrier molecules include protein, including keyhole limpet hemocyanin, which is a preferred carrier protein. The chimeric construct can be conjugated to the carrier molecule using standard methods. (Hancock et al., “Synthesis of Peptides for Use as Immunogens,” in Methods in Molecular Biology: Immunochemical Protocols, Manson (ed.), pages 23-32 (Humana Press 1992)).

In some embodiments, the present disclosure contemplates an immunogenic composition (e.g., vaccine composition) comprising a pharmaceutically acceptable injectable vehicle. The vaccines of the present disclosure may be administered in conventional vehicles with or without other standard carriers, in the form of injectable solutions or suspensions. The added carriers might be selected from agents that elevate total immune response in the course of the immunization procedure.

Liposomes have been suggested as suitable carriers. The insoluble salts of aluminum, that is aluminum phosphate or aluminum hydroxide, have been utilized as carriers in routine clinical applications in humans. Polynucleotides and polyelectrolytes and water-soluble carriers such as muramyl dipeptides have been used.

Preparation of injectable vaccines of the present disclosure, includes mixing the immunogenic composition (e.g., vaccine composition) with muramyl dipeptides or other carriers. The resultant mixture may be emulsified in a mannide monooleate/squalene or squalane vehicle. Four parts by volume of squalene and/or squalane are used per part by volume of mannide monooleate. Methods of formulating immunogenic compositions (e.g., vaccine compositions) are well-known to those of ordinary skill in the art. (Rola, Immunizing Agents and Diagnostic Skin Antigens. In: Remington's Pharmaceutical Sciences, 18th Edition, Gennaro (ed.), (Mack Publishing Company 1990) pages 1389-1404).

Additional pharmaceutical carriers may be employed to control the duration of action of a vaccine in a therapeutic application. Control release preparations can be prepared through the use of polymers to complex or adsorb chimeric construct. For example, biocompatible polymers include matrices of poly(ethylene-co-vinyl acetate) and matrices of a polyanhydride copolymer of a stearic acid dimer and sebacic acid. (Sherwood et al. (1992) Bio/Technology 10: 1446). The rate of release of the chimeric construct from such a matrix depends upon the molecular weight of the construct, the amount of the construct within the matrix, and the size of dispersed particles. (Saltzman et al. (1989) Biophys. J. 55: 163; Sherwood et al, supra.; Ansel et al. Pharmaceutical Dosage Forms and Drug Delivery Systems, 5th Edition (Lea & Febiger 1990); and Gennaro (ed.), Remington's Pharmaceutical Sciences, 18th Edition (Mack Publishing Company 1990)). The chimeric construct can also be conjugated to polyethylene glycol (PEG) to improve stability and extend bioavailability times (e.g., Katre et al.; U.S. Pat. No. 4,766,106).

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of a composition described herein may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of a compound described herein in a single dose. In some embodiments, an effective amount is the combined amounts of a compound described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof, in or on a subject. The composition of the immunogenic composition (e.g., vaccine composition) described herein may be administered systemically (e.g., via intravenous injection) or locally (e.g., via local injection). In some embodiments, the composition of the immunogenic composition (e.g., vaccine composition) described herein is administered orally, intravenously, topically, intranasally, or sublingually. Parenteral administration is also contemplated. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the composition is administered prophylactically.

In some embodiments, the composition or immunogenic composition (e.g., vaccine composition) is administered once or multiple times (e.g., 2, 3, 4, 5, or more times). For multiple administrations, the administrations may be done over a period of time (e.g., 1 week, 2 weeks, 3 weeks, 1 month, 2 months, 3 months, 4 months, 5 months, 6 months, 1 year, 2 years, 5 years, 10 years, or more than 10 years). In some embodiments, the composition or immunogenic composition (e.g., vaccine composition) is administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later).

### EXAMPLES

### Introduction

The dialogue between innate and adaptive branches of the immune system is a central paradigm of modern immunology and is critical for protection against infections as well as the pathogenesis of autoimmune, allergic and inflammatory diseases (Banchereau and Steinman, 1998; Iwasaki and Medzhitov, 2004; Janeway and Medzhitov, 2002; Matzinger, 1994). According to the current model, peripheral tissue infection and/or damage leads to activation and migration of innate immune phagocytes to the draining lymph node (dLN) where they initiate an antigen-dependent adaptive immune response. Alternatively, innate stimuli or microbes with specific physical properties (e.g., diameter in the nanometer range) can directly drain to the dLN and activate LN-resident innate and adaptive immune cells (Bachmann and Jennings, 2010; Irvine et al., 2020). The dLN has been thoroughly scrutinized for its capacity to host adaptive immune responses, but recent reports highlight that the antigen-dependent adaptive immune response is preceded and supported by an antigen-independent lymph node innate response (LIR) (Acton et al., 2014; Coccia et al., 2017; De Giovanni et al., 2020; Didierlaurent et al., 2014; Kastenmuller et al., 2012; Lian et al., 2020; Lynn et al., 2015; Martin-Fontecha et al., 2004; Soderberg et al., 2005; Wong et al., 2019; Wong et al., 2018; Xu et al., 2015b). Albeit incompletely characterized, LIR consists of at least two components that are both critical for the development of an effective adaptive immune response: antigen-independent LN expansion and establishment of a pro-inflammatory milieu (Acton and Reis e Sousa, 2016; Grant et al., 2020). It remains a mystery if, and how, the LIR differs when it is driven by phagocyte migration from the periphery, or when it is governed by the direct targeting of LN-resident innate immune cells.

Innate immune cells recognize pathogen-associated molecular patterns (PAMPs) through pattern recognition receptors (PRRs) (Janeway and Medzhitov, 2002). Several classes of PRRs have been identified so far, namely Toll-like receptors (TLRs), RIG-I-like receptors, cytosolic DNA sensors, NOD-like receptors and C-type lectin receptors (CLRs) (Brubaker et al., 2015). PRR activation within innate immune cells is critical for triggering inflammation and the ensuing development of adaptive immune responses. For this reason, PRR targeting has been harnessed for vaccine development (O'Hagan et al., 2020). Adjuvant formulations including TLR4 and TLR9 ligands (respectively monophosphoryl lipid A (MPL) and CpG) promote robust and long-lasting adaptive immune responses and became adjuvant components of FDA-approved vaccines. Several additional PRR ligands have been tested in pre-clinical models of immunization and are currently being evaluated in clinical trials (O'Hagan et al., 2020). Among PRRs, the biology of CLRs and their potential as vaccine adjuvant targets have been less investigated.

The CLR superfamily comprises hundreds of proteins that share carbohydrate-binding domains and play a key role in the development of innate and adaptive immune responses to fungal infection through recognition of cell wall polysaccharides (Borriello et al., 2020; Brown et al., 2018). Pre-clinical and clinical data support the concept of targeting the CLR Mincle to elicit robust cellular and humoral immunity upon vaccination (Pedersen et al., 2018). In addition, there is evidence that activation of the CLRs Dectin-1 (Clec7a) and Dectin-2 (Clec4n) by their respective polysaccharide ligands β-glucans and mannans promote antigen-specific adaptive immunity in experimental models of immunization (Petrovsky and Cooper, 2011). However, the mechanism of action of Dectins remains largely overlooked. Further complicating the mechanistic analyses, fungal polysaccharides like β-glucans and mannans vary by chemical structure and physical form (e.g., dimension and solubility). Physical properties of PRR ligands not only impact their localization (peripheral retention vs LN drainage) but also dictate recognition by and activation of innate immune cells. Dectin-1 and Dectin-2 bind fungal polysaccharides in soluble as well as insoluble forms but only the latter induces efficient receptor clustering and activation (Goodridge et al., 2011; Zhu et al., 2013). Consequently, it is widely believed that only particulate polysaccharides are capable of immuno-stimulation.

In this study this paradigm was re-examined and it was found that soluble mannans, while largely inactive in vitro and in vivo at the injection site, traffic to the lymph node due to their diameter and elicit a potent LIR. Remarkably, the responses observed bypass the need for dendritic cell migration from the periphery to the dLN. It was also demonstrated that further modulation of the physical properties of mannans results in novel immunological properties, such as full activation of innate immune cells in vitro and simultaneous targeting of the periphery and the LN. When tested as an adjuvant system formulated with viral glycoprotein antigens (influenza A virus (IAV) hemagglutinin or SARS-CoV-2 Spike), mannans enabled induction of neutralizing antibodies with broad epitope specificity and protected against infection with IAV or SARS-CoV-2, respectively. Overall, the data demonstrate that the quality of LN innate and adaptive immune responses can be tuned by modulating the physical properties of mannans, supporting important implications for adjuvant design and vaccine development.

### Results

Mannans Elicit LN-Restricted IFN Signatures that Drive LN Expansion

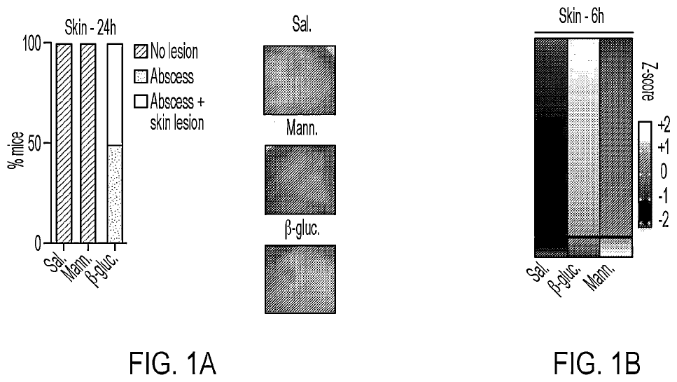

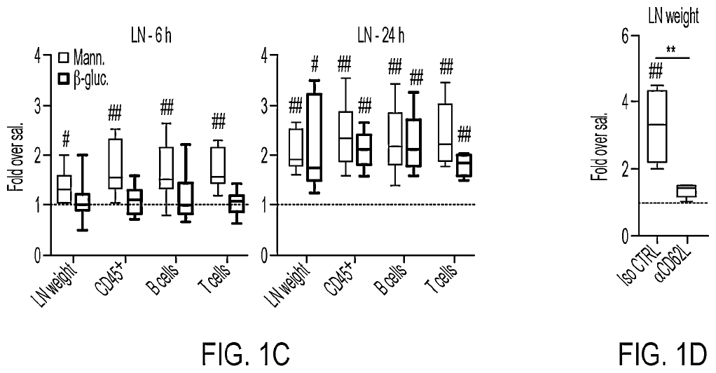

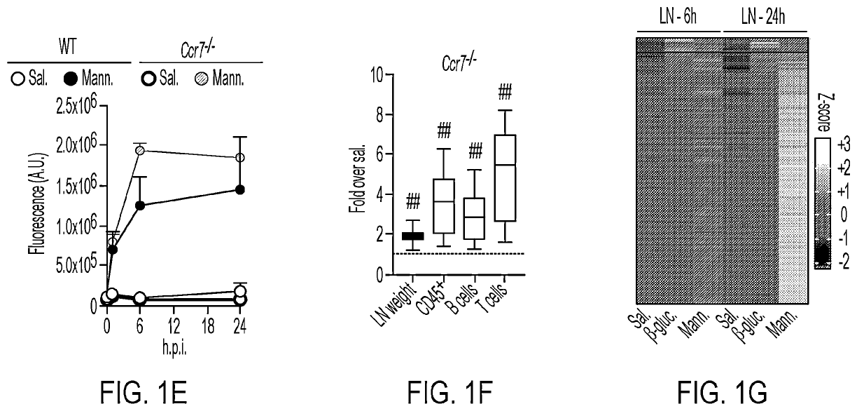

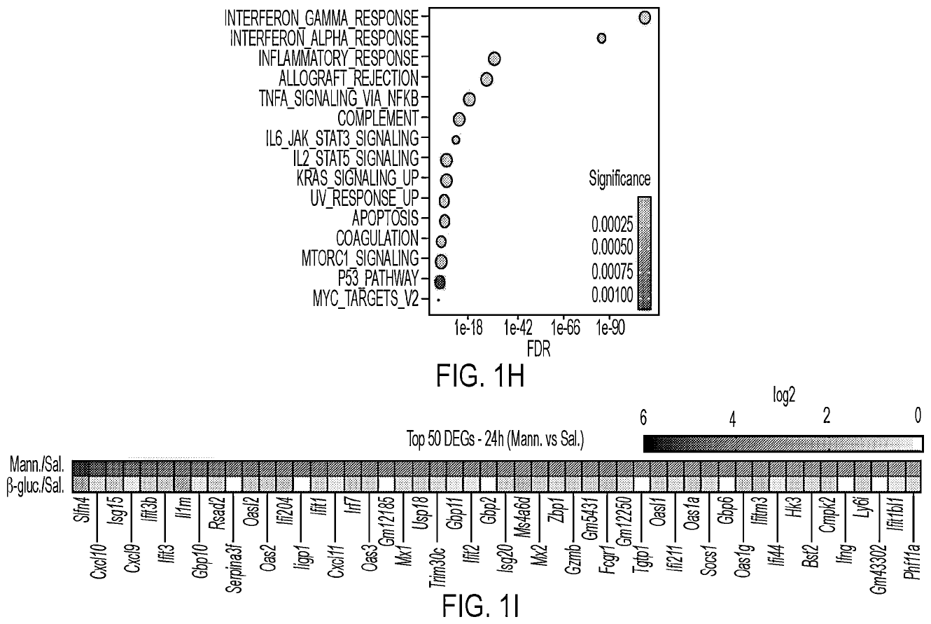

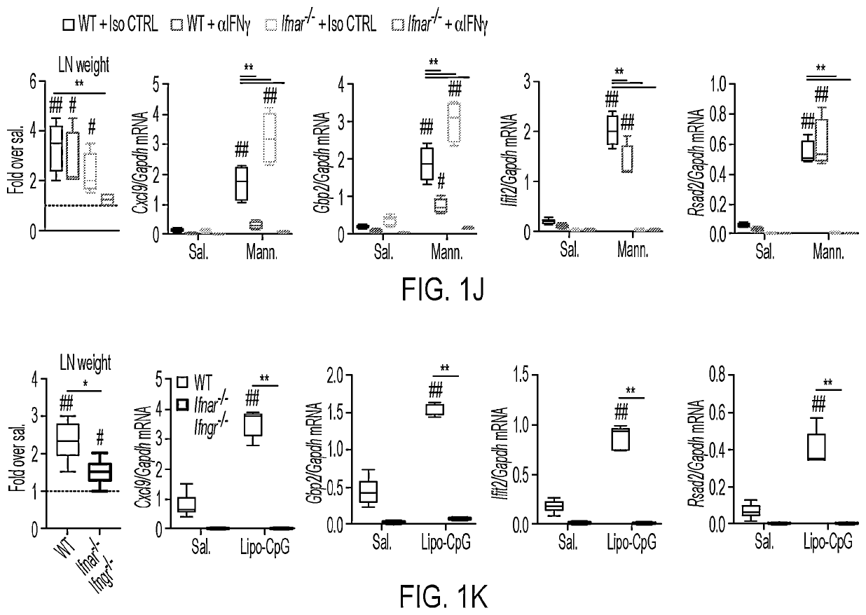

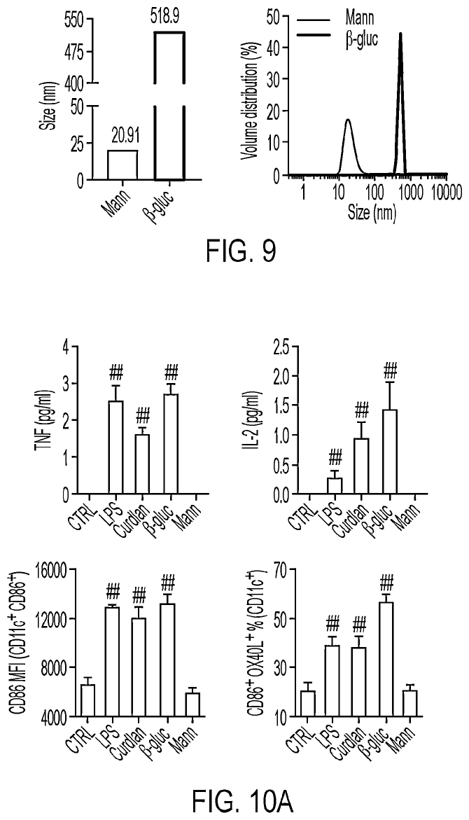

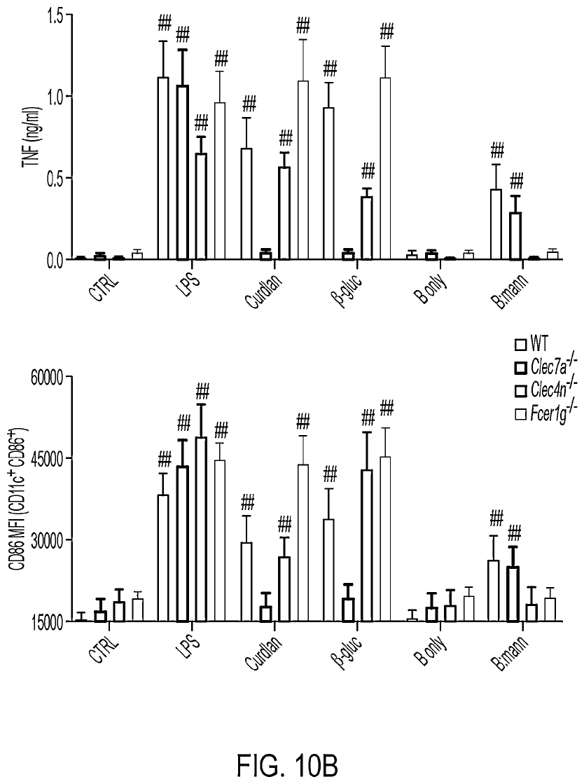

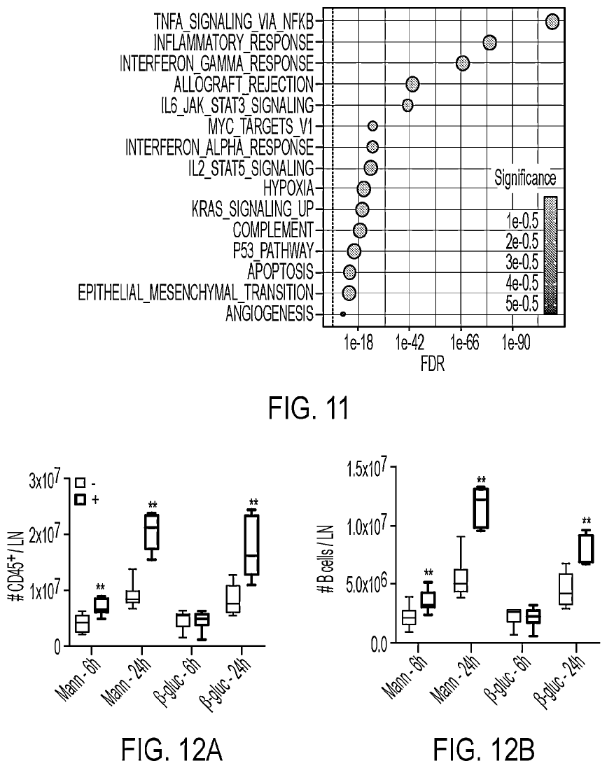

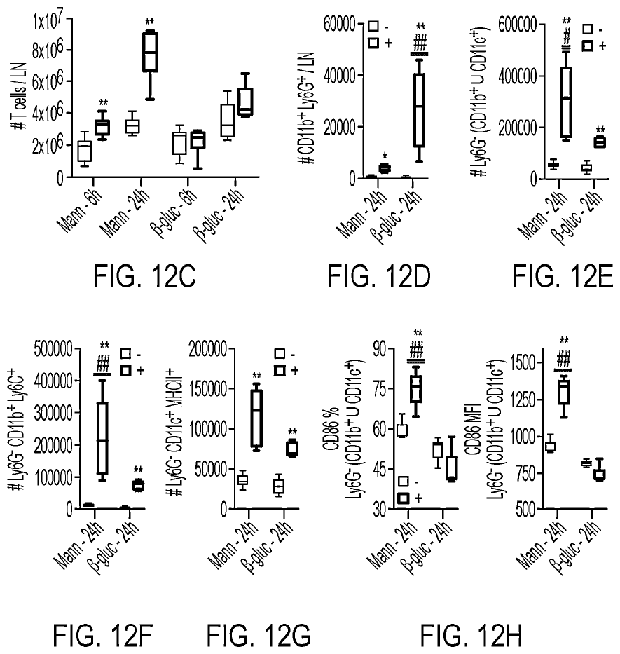

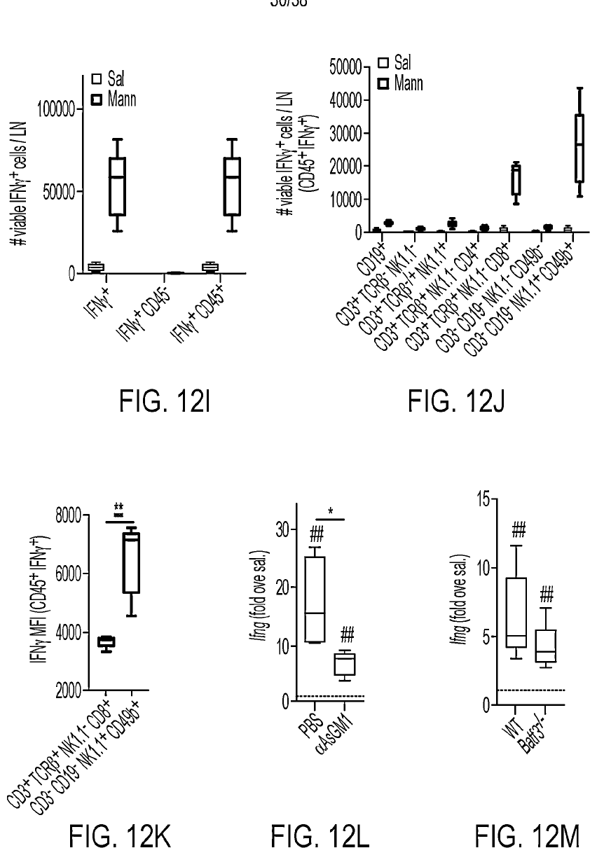

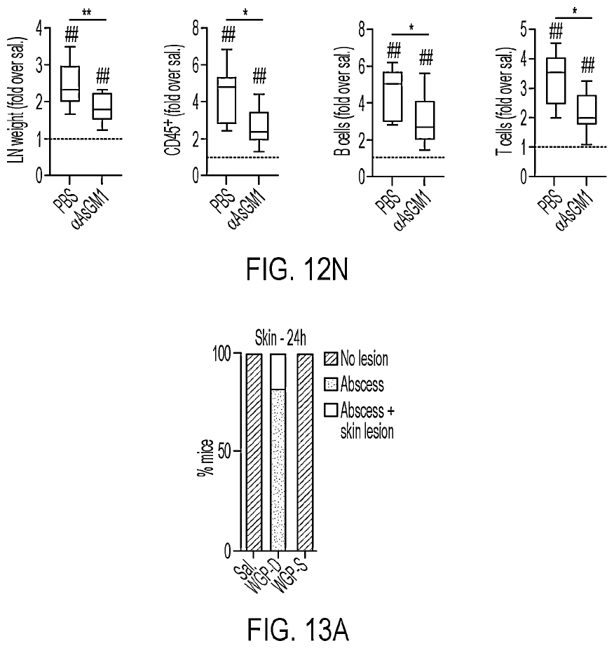

Dectin-1 and -2 bind the fungal cell wall polysaccharides β-glucans and mannans and both activate the kinase Syk either directly (Dectin-1) or through the FcRγ chain (Dectin-2) (Borriello et al., 2020; Lionakis et al., 2017; Netea et al., 2008). Preparations of β-glucans and mannans isolated from Candida albicans were employed, that exhibit distinct physical forms being respectively insoluble with a diameter of ˜500 nm and soluble with a diameter of ˜20 nm (FIG. 9). In keeping with the current model of dectin activation (Goodridge et al., 2011; Zhu et al., 2013), particulate β-glucans, but not soluble mannans, were able to elicit cytokine production and co-stimulatory molecule expression by GM-CSF-differentiated bone marrow-derived phagocytes (FIG. 10A). As expected, β-glucans signaling required Dectin-1 (FIG. 10B). In contrast to soluble mannans, immobilization of mannans onto microbeads resulted in Dectin-2 and FcRγ-dependent phagocyte activation (FIG. 10B, lipopolysaccharide (LPS) and curdlan were used as controls). Accordingly, β-glucans elicited formation of skin abscesses and lesions upon in vivo intradermal injection whereas mannans did not induce signs of skin inflammation (FIG. 1A). These results were confirmed by transcriptomic analysis of skin samples injected with saline, β-glucans and mannans (FIG. 1B). Pathway analysis of the cluster of differentially expressed genes (DEGs) upregulated by β-glucans revealed enrichment for pro-inflammatory and type II interferon (IFN) pathways, consistent with the innate immune response elicited by C. albicans skin infection (FIG. 11) (Santus et al., 2017). While soluble mannans did not induce skin inflammation, these fungal ligands induced dLN expansion and lymphocyte accrual as early as 6 hours post-injection (h.p.i.) which was sustained at 24 h.p.i. (FIGS. 1C and 12A-12C) and dependent on circulating leukocyte recruitment (FIG. 1D). β-glucans also elicited LN expansion but only at 24 h.p.i. (FIGS. 1C, 12A-12C). Both mannans and β-glucans increased myeloid cell numbers in the dLN (FIGS. 12D-12G), with β-glucans preferentially increasing neutrophil numbers possibly due to neutrophil drainage from the skin injection site (FIG. 12D). However, only mannans were able to induce myeloid cell activation as measured by increased CD86 expression (FIG. 12H). Thus, β-glucans and mannans differ in their ability to induce local versus dLN inflammatory responses, with the latter inducing LIR at a faster rate than the former. Considering the kinetics of response to mannans and their diameter (compatible with lymphatic drainage), it was reasoned that mannans might activate LN-intrinsic circuits that eventually lead to dLN expansion. In keeping with this hypothesis, mannans rapidly accumulated to the dLN (within 6 h.p.i.) (FIG. 1E) and were able to induce a dLN expansion even in Ccr7^{-/-} mice (FIG. 1F) in which migration of immune cells from the periphery (i.e. skin) to the dLNs is abolished (Ohl et al., 2004). Collectively, these data demonstrate that mannans activate a potent LIR in the absence of peripheral (skin) inflammation and/or phagocyte migration to the dLN. Transcriptomic analysis of dLNs isolated from saline-, β-glucan- and mannan-injected mice was conducted to further characterize the molecular events associated with LN expansion. A completely opposite profile was found compared to the skin, with mannans eliciting an earlier and more pronounced transcriptional response compared to β-glucans (FIG. 1G). Pathway analysis revealed that the cluster of DEGs upregulated by mannans is enriched for type I and II IFN pathways (FIG. 1H), as confirmed by the expression levels of the top 50 genes upregulated in mannan-treated dLN compared to controls (FIG. 1I).

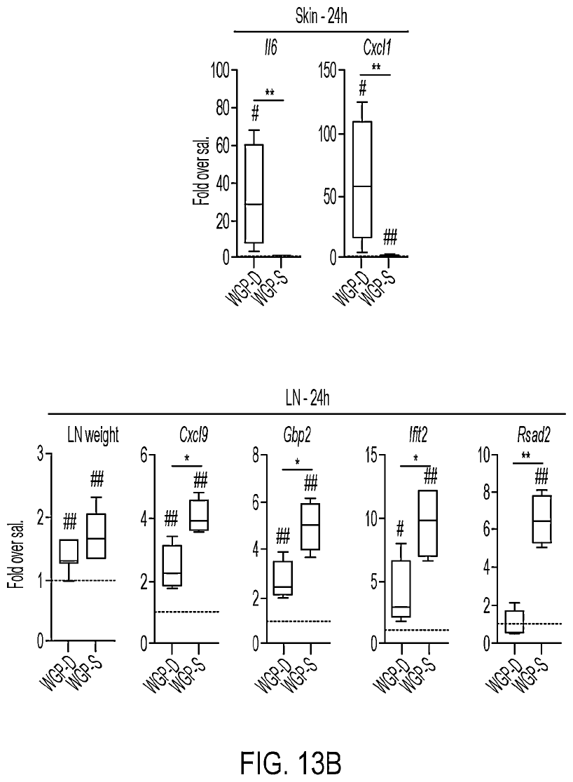

The presence of type II IFN (IFNγ)-producing cells in mannan-treated dLN was confirmed by flow cytometry (FIG. 12I). It was found that that majority of IFNγ-producing cells were CD8⁺ T and NK cells (FIG. 12J), with NK cells expressing IFNγ at higher levels (FIG. 12K). To assess if NK cells were the major source of IFNγ in mannan-treated dLN, NK cells were depleted and impairment of mannan-elicited Ifng expression was observed (FIG. 12L). cDC1 produce cytokines that induce NK cell activation and IFNγ production (Cancel et al., 2019). They may therefore contribute to mannan-induced LIR. To assess this hypothesis, Batf3^{-/-} mice that lack cDC1 were used and no impairment of mannan-elicited Ifng expression was observed (FIG. 12M). Notably, NK cell depletion only partially affected LN expansion and cell accrual (FIG. 12N). In keeping with this, IFNγ blockade only partially reduced the expansion of LN following mannan injection (FIG. 1J). Furthermore, simultaneous blockade of type I and type II IFNs (using Ifnar^{-/-} mice and an anti-IFNγ blocking antibody) prevented mannan-elicited LN expansion and induction of ISGs (FIG. 1J). This mechanism was not restricted to mannans since LN expansion and ISG induction elicited by Lipo-CpG, a well characterized LN-targeted TLR9 ligand (Liu et al., 2014), were also impaired in Ifnar^{-/-} Ifngr^{-/-} mice (FIG. 1K). Therefore, the results demonstrate that soluble mannans isolated from C. albicans elicit a LIR that differs from the one induced by β-glucans, namely in the induction of ISGs. To further establish whether these differences are due to distinct physical forms of fungal polysaccharides, the response to whole glucan particles (WGP) was evaluated in dispersible (D) or soluble (S) forms which have been respectively characterized as Dectin-1 agonists and antagonists (Goodridge et al., 2011). Consistent with the pattern observed with mannans, WGP-S did not induce skin inflammation but elicited LN expansion and ISG expression (FIGS. 13A-13B). Altogether, these results support a model in which the physical form of fungal polysaccharides drive a LIR characterized by dLN expansion and ISG induction.

### Mannan-Elicited LIR Requires Dectin-2-Expressing, CD169+ Sinus Macrophages

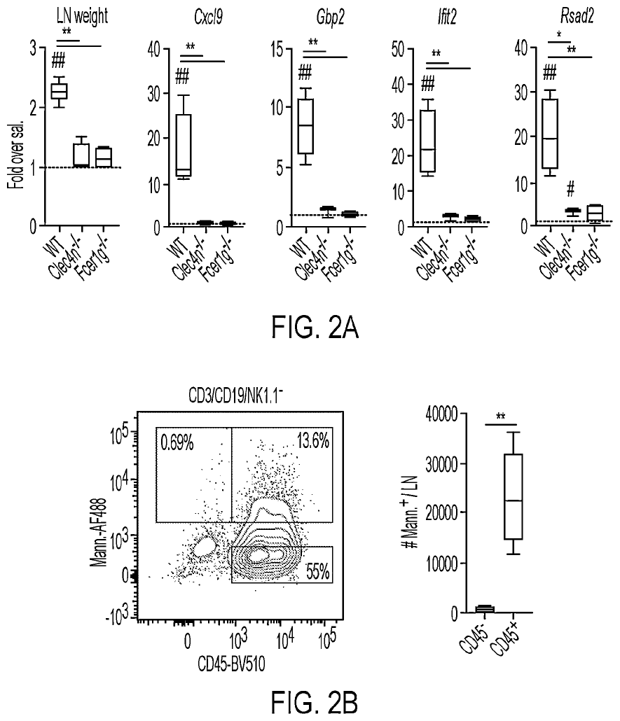

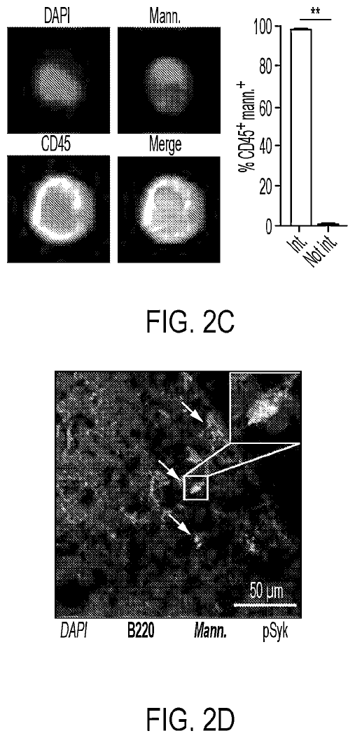

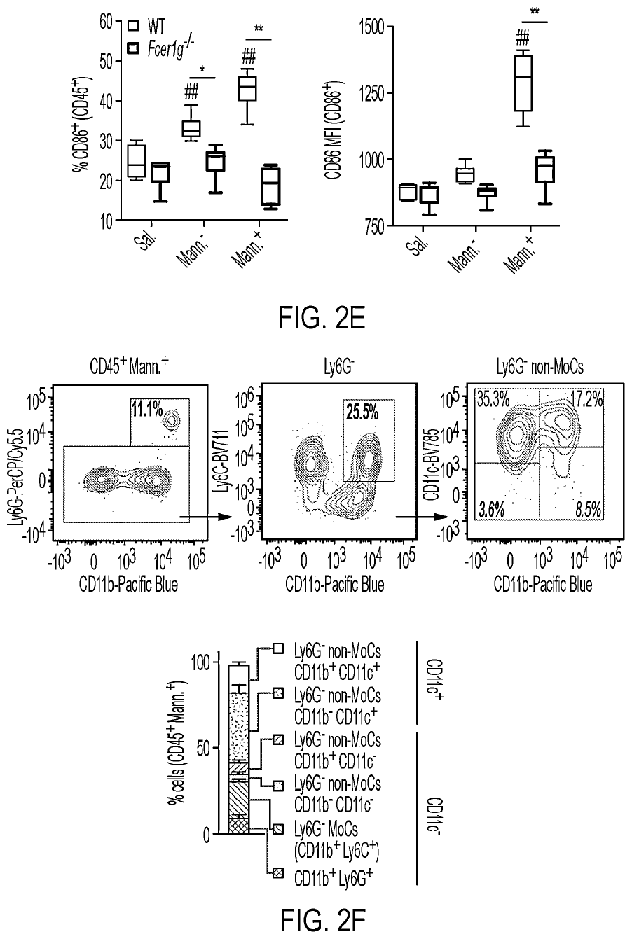

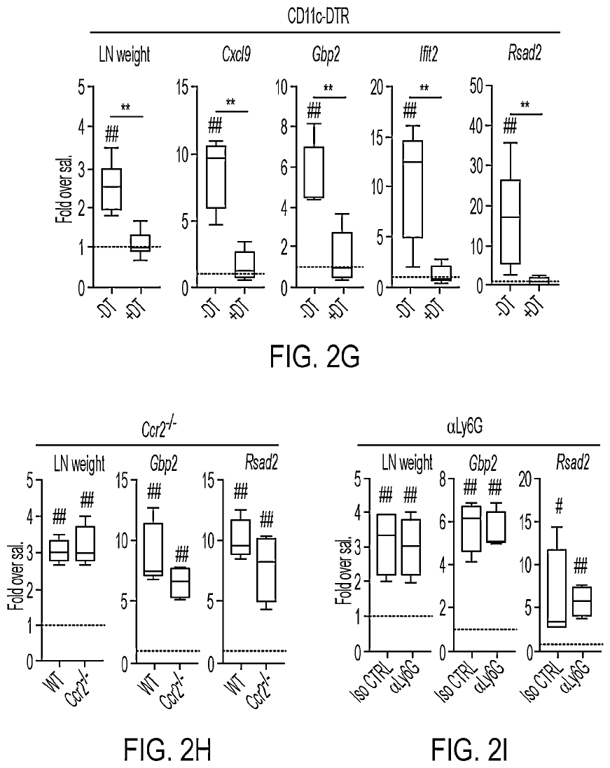

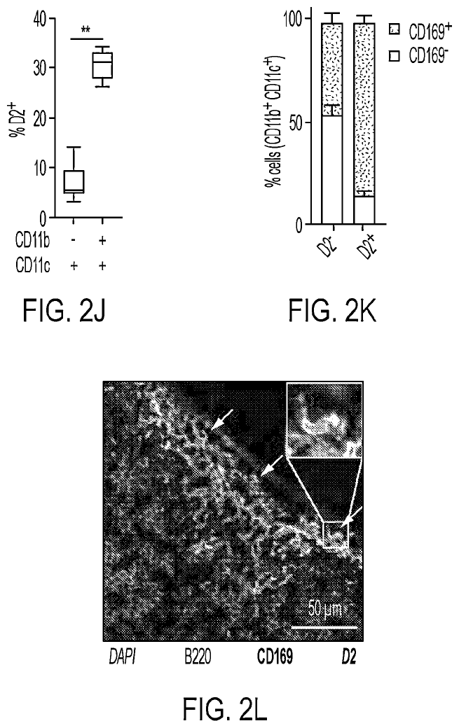

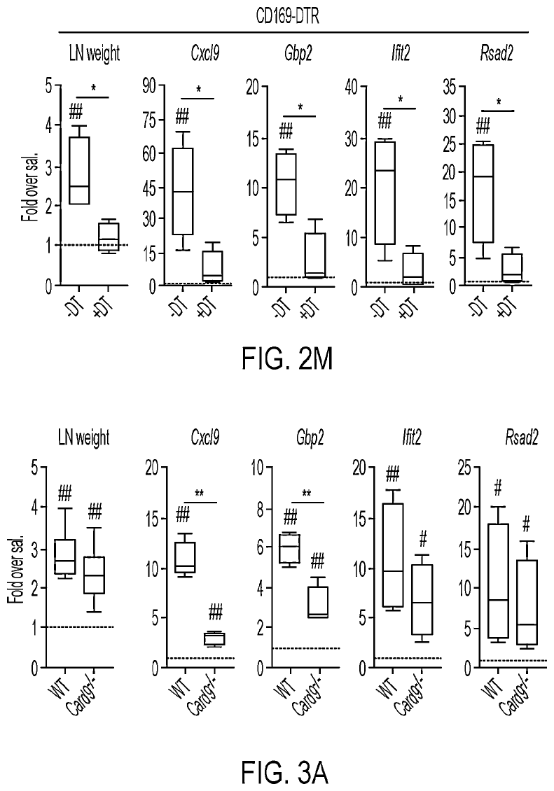

Dectin-2 is the major receptor for mannans (Borriello et al., 2020; Lionakis et al., 2017; Netea et al., 2008) and, in agreement with in vitro data, it was found that Dectin-2 and FcRγ were also required for mannan-elicited LIR (FIG. 2A). Dectin-2 is expressed mainly by myeloid cells (Taylor et al., 2005). Therefore, fluorescently labelled mannans were employed to enable immunophenotypic analysis of the non-lymphoid (CD3/CD19/NK1.1⁻) compartment of dLNs and identify cellular targets of mannans. The vast majority of mannan-laden cells were CD45⁺ cells (FIG. 2B). Imaging cytometry analysis confirmed that these cells internalized mannans (FIG. 2C). In addition, confocal microscopy analysis of dLNs 1 h.p.i. of mannans showed colocalization of phospho-Syk and mannans (FIG. 2D), which is indicative of Dectin-2/FcRγ-mediated activation (Borriello et al., 2020). Accordingly, mannan-laden cells exhibited the highest levels of expression of CD86, marker of innate immune cell activation, in an FcRγ-dependent manner (FIG. 2E). The phenotype of mannan-laden cells was further characterized and it was found that more than 50% were Ly6G⁻ (CD11b⁺Ly6C⁺)⁻ CD11c⁺ cells, while less abundant cell subsets were neutrophils (CD11b⁺ Ly6G⁺) and monocytes/monocyte-derived cells (MoCs, Ly6G⁻ CD11b⁺ Ly6C⁺) (FIG. 2F). Interestingly, using diphtheria toxin (DT)-mediated depletion of CD11c⁺ cells in CD11c-DT receptor (DTR) mice, abolished mannan-elicited LIR was abolished (FIG. 2G). On the other hand, mannan-elicited LIR was not affected in Ccr2^{-/-} mice (in which monocyte egress from the bone marrow is impaired) (FIG. 2H) or by antibody-mediated depletion of neutrophils (FIG. 2I). CD11c⁺ cells can be further distinguished based on the expression of CD11b (FIG. 2F). Since Dectin-2 is critical for mannan-elicited LIR, its expression was assessed on CD11b-CD11c⁺ and CD11b⁺CD11c⁺ cells at steady state. Dectin-2 was mainly expressed by CD11b⁺CD11c⁺ cells (FIG. 2J), and the majority of CD11b⁺CD11c⁺ Dectin-2⁺ cells expressed the subcapsular and medullary sinus macrophage marker CD169 (FIG. 2K). Confocal microscopy analysis confirmed colocalization of CD169 and Dectin-2 on cells lining the LN subcapsular sinus (FIG. 2L). Furthermore, DT-mediated depletion of CD169⁺ cells in CD169-DTR mice phenocopied the results obtained with CD11c-DTR mice and completely abolished mannan-elicited LIR (FIG. 2M). These results demonstrate a role for CD169⁺ sinus macrophages as sentinels of lymph-borne materials (Moran et al., 2019).

### Activation of the Non-Canonical NF-kB Subunit RelB Governs Mannan-Elicited LIR

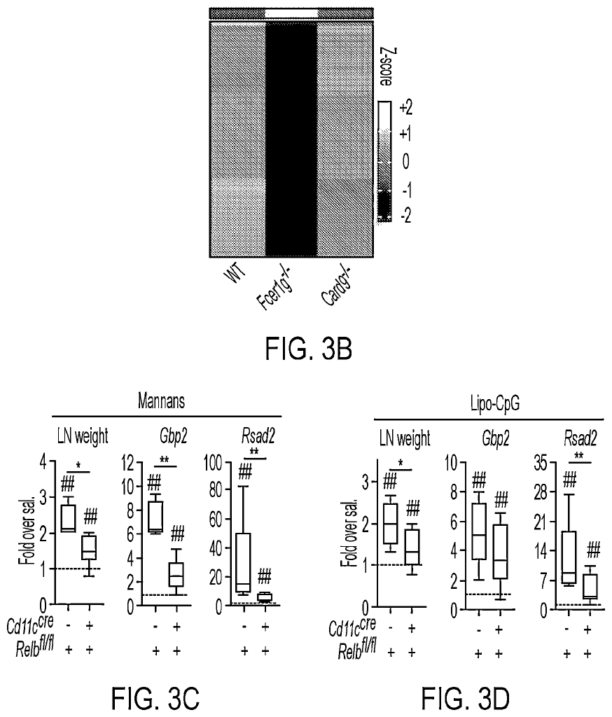

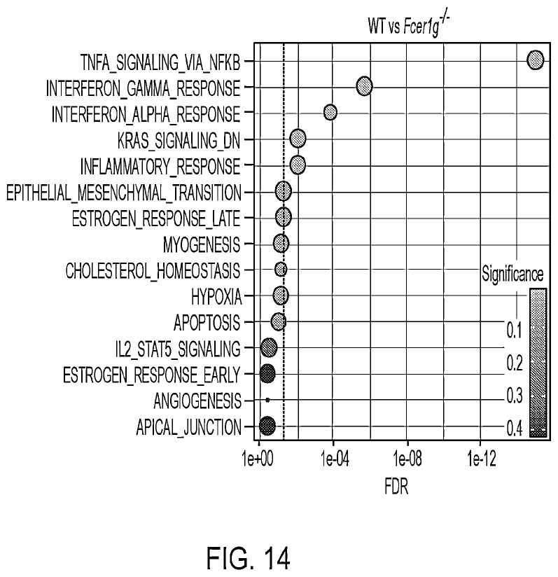

The evidence of Syk phosphorylation in LN-resident, mannan-laden cells as early as 1 h.p.i. points to activation of signaling pathways downstream of Dectin-2. A key step in the Dectin receptor-Syk pathway is the activation of CARD9 which in turn regulates the activity of several signaling molecules and transcription factors, including MAPKs and canonical NF-κB (Borriello et al., 2020). Therefore, Card9^{-/-} mice were employed to characterize the molecular events associated with mannan-elicited LIR. Surprisingly, mannan-elicited LN expansion was comparable between wild type (WT) and Card9^{-/-} mice (FIG. 3A). In keeping with the relevance of the IFN signature in driving the LIR, ISG induction was completely abrogated in Clec4n^{-/-} and Fcer1g^{-/-} mice (FIG. 2A), but it was largely maintained in Card9^{-/-} mice (FIG. 3A). In particular, type I IFN-dependent genes were unchanged in Card9^{-/-} compared to WT mice, while type II IFN-dependent ISGs, although significantly decreased compared to WT mice, were still partially induced (FIG. 3A). To gain further insights into the molecular events associated with CARD9-dependent and -independent activation of mannan-laden cells, targeted transcriptomic analysis of mannan-laden CD11b⁺CD11c⁺ cells isolated from WT, Fcer1g^{-/-} and Card9^{-/-} mice was performed. While several genes were differentially expressed between WT or Card9^{-/-} and Fcer1g^{-/-} mice, cells isolated from WT and Card9^{-/-} mice exhibited strikingly similar transcriptomes (FIG. 3B). Pathway enrichment analysis revealed that DEGs between cells isolated from WT and Fcer1g^{-/-} are represented in TNF/NF-κ B, type I and II IFN pathways (FIG. 14). To explain this phenotype, it was reasoned that in the model the Dectin-2-Syk axis might activate CARD9-independent pathways. Of note, Syk activates the kinase NIK which in turn leads to the CARD9-independent activation of the non-canonical NF-κB transcription factor RelB (Gringhuis et al., 2009; Xu et al., 2018). Therefore, mice were generated in which RelB is conditionally deleted in the CD11c⁺ compartment (Cd11c^{cre} Relb^{fl/fl}). Notably, it was found that mannan-induced LN expansion and expression of both type I and type II IFN-dependent ISGs were significantly reduced compared to control (Relb^{fl/fl}) mice (FIG. 3C). To verify whether RelB requirement was restricted to mannans Lipo-CpG was employed and it was found that LN expansion and type I IFN-dependent ISG expression elicited by Lipo-CpG were impaired in Cd11c^{cre} Relb^{fl/fl} mice (FIG. 3D). These results support a model in which RelB regulates LN expansion and type I IFN-dependent ISG expression elicited by LN-targeted stimuli, and further cooperates with CARD9-dependent pathways to regulate mannan-induced type II IFN-dependent ISG expression.

### Molecular Pathways Required for Mannan-Elicited LIR Regulate the Magnitude of Mannan Adjuvant Activity

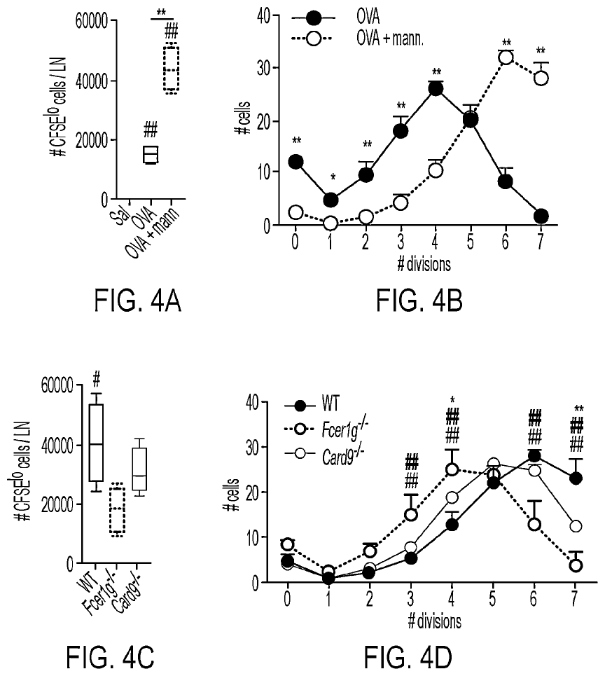

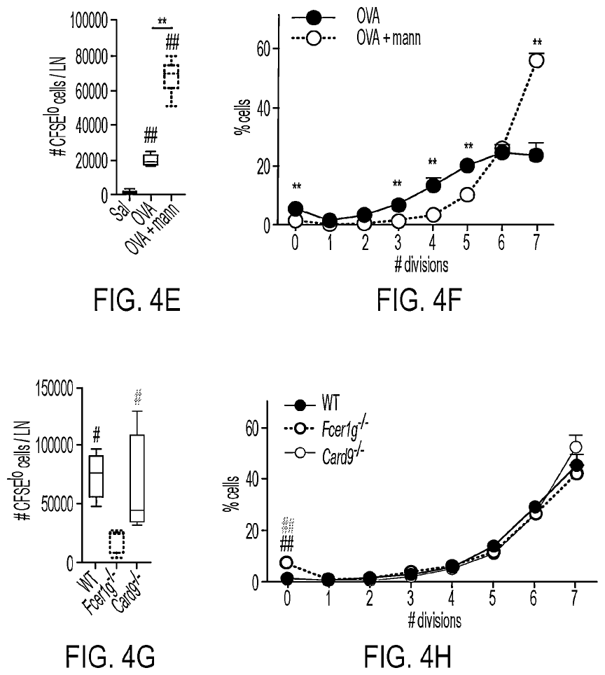

There is evidence suggesting that the LIR is essential to control the development of adaptive immune responses (Coccia et al., 2017; De Giovanni et al., 2020; Lynn et al., 2015; Martin-Fontecha et al., 2004; Soderberg et al., 2005). It was reasoned that lymphocyte accrual and IFN signatures induced by mannan may favor the encounter of T cells with their cognate antigen and its efficient presentation by the innate immune compartment, thereby improving the adaptive immune response. A model of adoptive transfer of CFSE-labelled, OVA-specific OT-II CD4⁺ T cells was employed to assess modulation of T cell proliferation (FIGS. 4A-4H). Combining mannans with OVA resulted in a strong increase in the numbers of OT-II and OT-I cells in the dLN compared to mice injected with saline or OVA alone (FIGS. 4A and 4E). This increase was likely due to higher OT-II CD4+ T cell recruitment to the dLN and more efficient antigen presentation/co-stimulation by LN-resident innate immune cells. Indeed, a lower percentage of non-proliferating T cells was detected, as well as a higher percentage of T cells undergoing 6 or 7 divisions in mice treated with OVA and mannans compared to OVA alone (FIGS. 4B and 4F). Of note, the effect of mannans on T cell proliferation was abrogated in Fcer1g^{-/-} mice while it was only attenuated in Card9^{-/-} mice (FIGS. 4C, 4D, 4G, and 4H). These results show that pathways required for mannan-elicited LIR are also critical for the adjuvant activity of mannans on antigen-specific adaptive immune responses.

Mannans Formulated with Aluminum Hydroxide Acquire Novel Physical Properties that Predict Immunological Functions

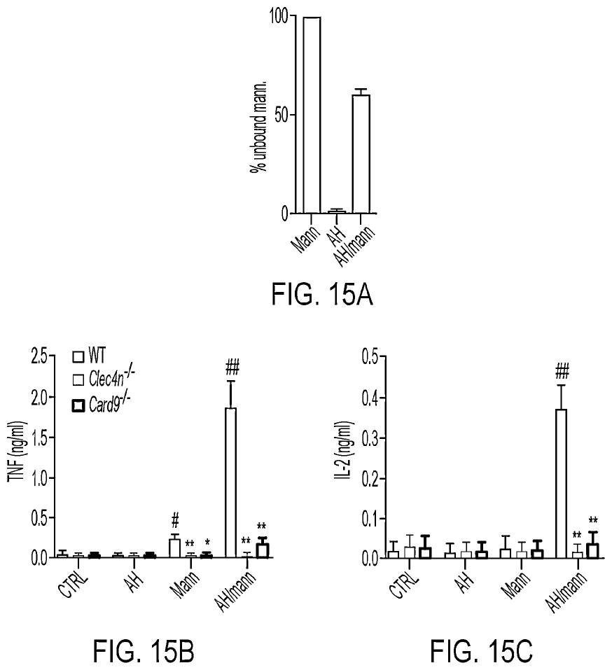

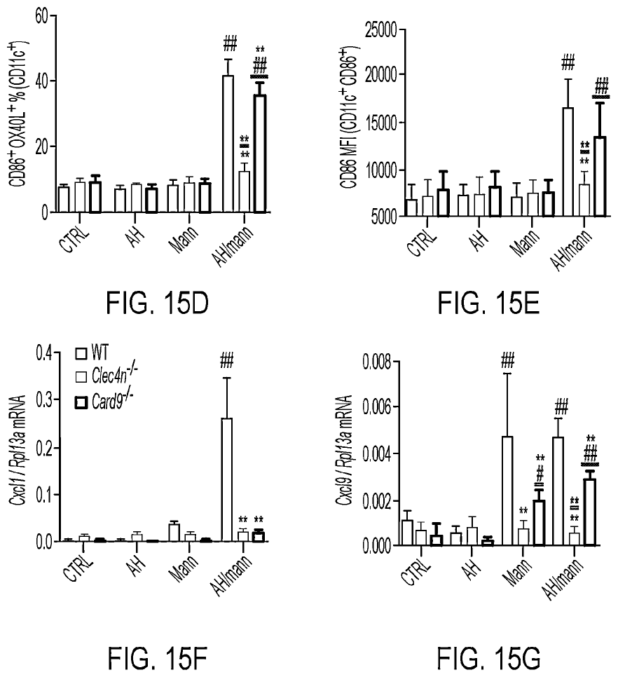

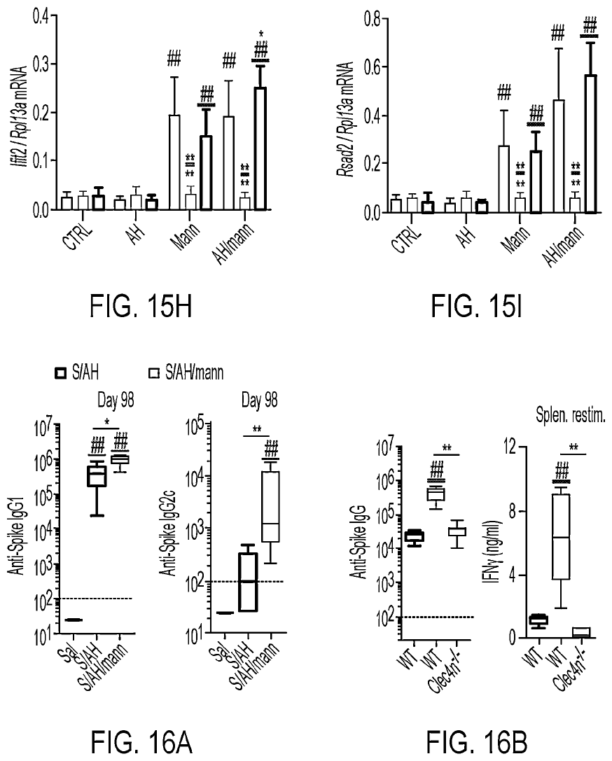

To further modulate the physical properties of mannans, it was reasoned that the presence of phosphate groups in mannan preparations might promote adsorption onto aluminum hydroxide (AH) as has been shown for other molecules (Morefield et al., 2005; Moyer et al., 2020). To characterize the formulation of mannans with AH, ¹H-NMR analysis was performed on supernatant collected after the adsorption of mannans onto AH. It was found that ˜40% of mannans adsorbed onto AH, while the remaining part remained soluble (FIG. 15A). It was also quantified that AH bound mannans at approximately two times its mass in the formulation used in these experiments.

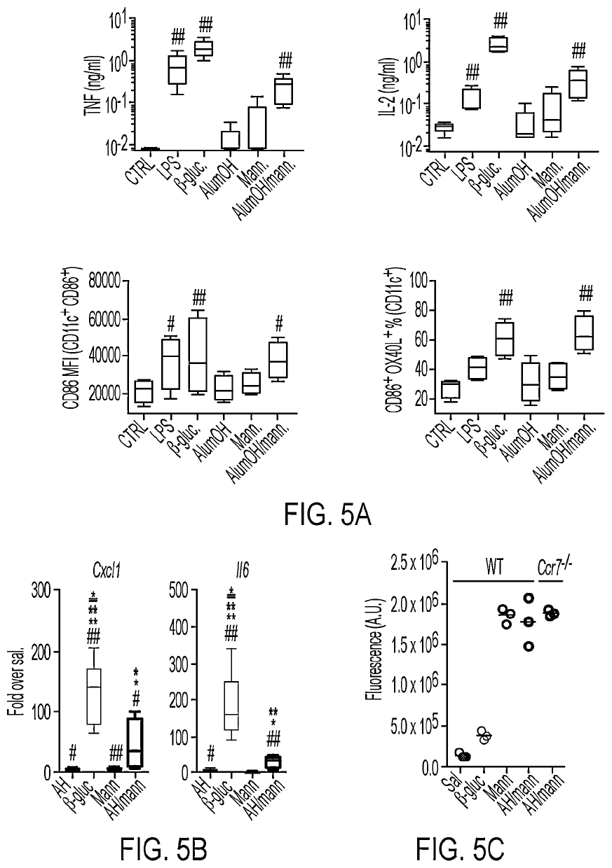

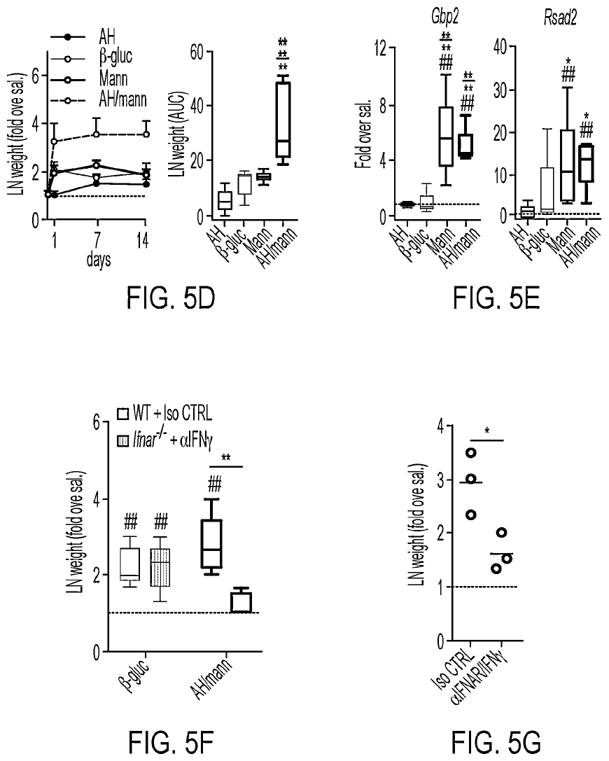

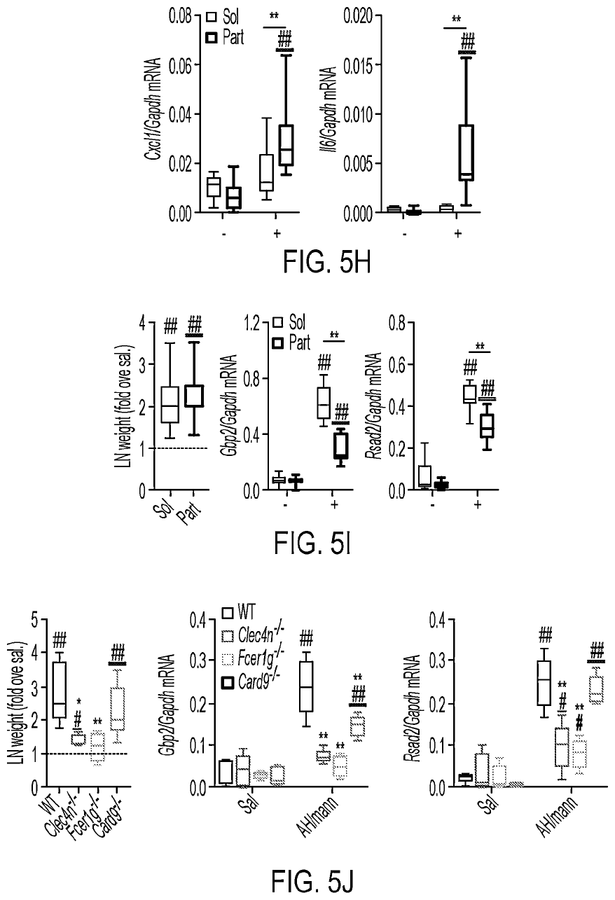

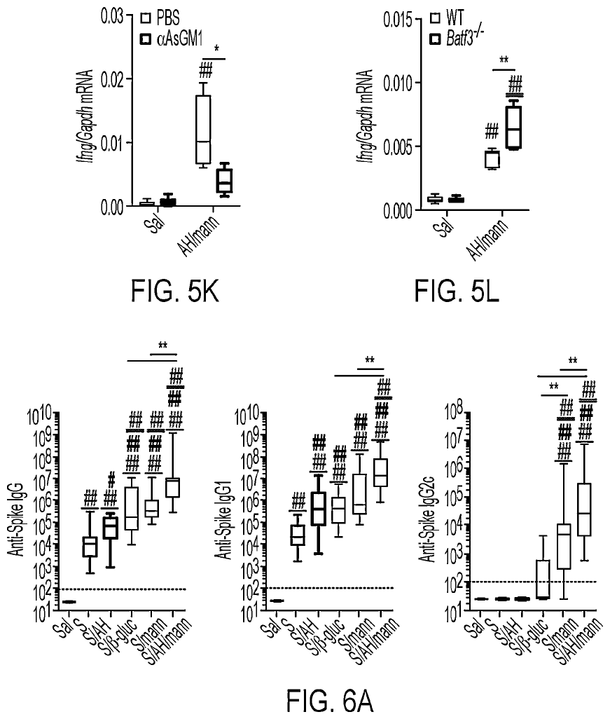

In vitro experiments with GM-CSF-differentiated bone marrow-derived phagocytes revealed that formulation of mannans with AH (AH/mann) completely changed their pro-inflammatory activity compared to soluble mannans. Mannans formulated with AH (alumOH/mann) induced significant co-stimulatory molecule expression and cytokine production (FIG. 5A). AH/mann, but not AH or mannans alone, induced significant TNF and IL-2 cytokine production in a Dectin-2- and CARD9-dependent manner (FIGS. 15B and 15C). AH/mann also elicited costimulatory molecule expression in a Dectin-2-dependent but CARD9-independent manner (FIGS. 15D and 15E). Finally, both mannans and AH/mann, but not AH alone, induced ISG expression in a Dectin-2-dependent but CARD9-independent manner, with the exception of CXCL1 that was significantly upregulated only in response to the AH/mann in WT cells (FIGS. 15F-15I).

When injected into mice, the AH/mann formulation resulted in phenotypes that were similar to the combined actions of glucans and mannans. In particular, AH/mann elicited skin inflammation (a phenotype observed for glucans but not soluble mannans) (FIG. 5B), but also drained to the LN in a CCR7-independent manner (FIG. 5C). Assessment of LN weight over time revealed that AH/mann induced a higher cumulative LN expansion compared to AH, mannans or β-glucans (FIG. 5D). Moreover, mannans and AH/mann elicited comparable ISG expression in the dLN 24 h.p.i. (FIG. 5E). As expected, β-glucans induced LN expansion but were largely excluded from the LN and did not induce ISG expression (FIGS. 5C-5E). In addition, LN expansion induced by AH/mann, but not β-glucans, was impaired in Ifnar^{-/-} mice treated with an anti-IFNγ blocking antibody (FIG. 5F). Similar results were obtained when IFN production was transiently blocked by treating wild type mice with anti-IFNAR and anti-IFNγ blocking antibodies (FIG. 5G). Overall, these results are compatible with a model in which the particulate fraction (mannans adsorbed onto AH) promotes skin inflammation while the soluble fraction (unbound mannans) drains to the LN and induces the ISG expression that was observed with the AH/mann formulation. Side by side injections of mice with either particulate or soluble fractions of AH/mann validated this model. As expected, the particulate and soluble fractions respectively induced skin inflammation (FIG. 5H) and potent LN expression of ISGs (FIG. 5I). Finally, it was assessed whether AH/mann-induced LIR requires the same cellular and molecular mechanisms as mannan-induced LIR or if the immunomodulatory functions of AH previously described (Eisenbarth et al., 2008) completely rewired these requirements. AH/mann-elicited LN expansion and ISG induction were found to be impaired in mice lacking the Dectin-2 receptor complex (i.e. Clec4n^{-/-} and Fcer1g^{-/-}) (FIG. 5J). In addition, AH/mann-induced Ifng expression in the dLN was reduced in mice treated with the NK cell-depleting anti-Asialo-GM1 antibody (FIG. 5K) and preserved in Batf3^{-/-} mice lacking cDC1 (FIG. 5L), as observed with mannan-induced LIR. Overall, formulation with AH endows mannans with empowered immunological functions that can be predicted based on their physical properties (i.e., particulate vs soluble) and reflect triggering of mannan-dependent Dectin-2-activated pathways.

Immunization with SARS-CoV-2 Spike Protein and Aluminum Hydroxide/Mannans Generates Anti-Spike Type 1 Immunity and Cross-Reactive Neutralizing Antibodies with Broad Epitope Specificity

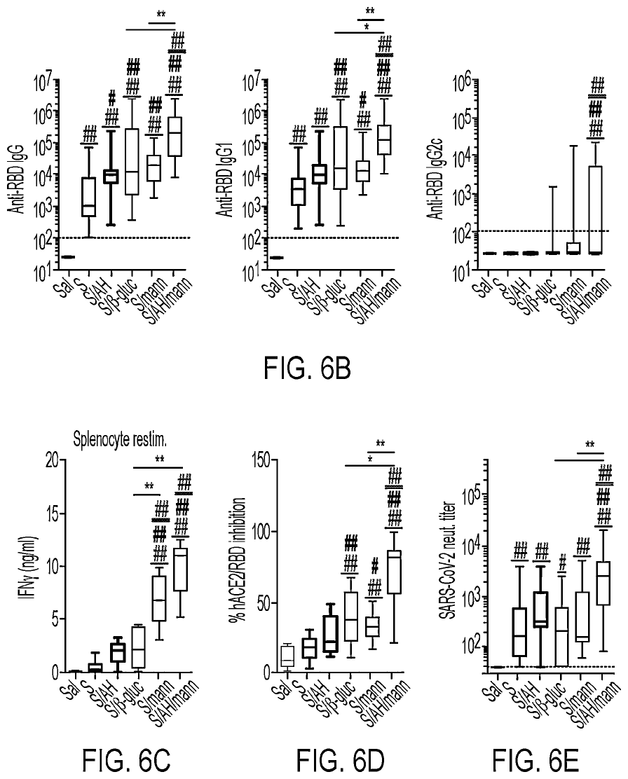

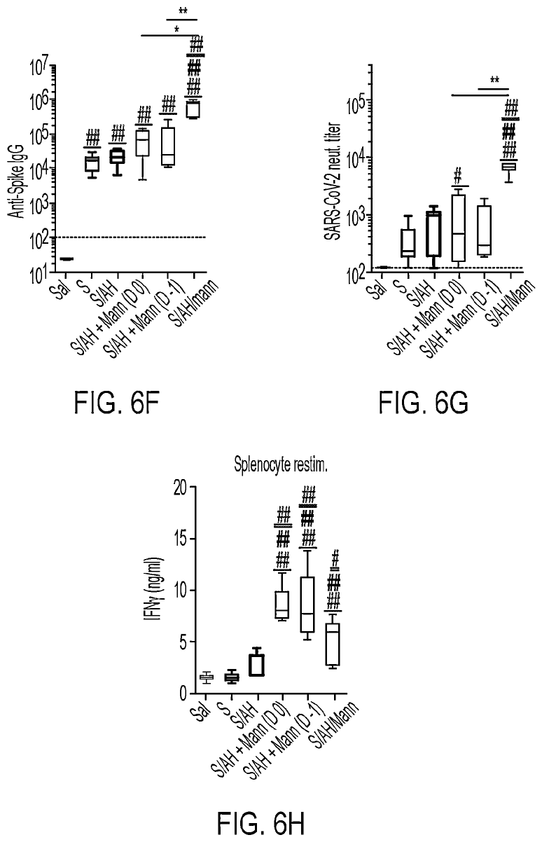

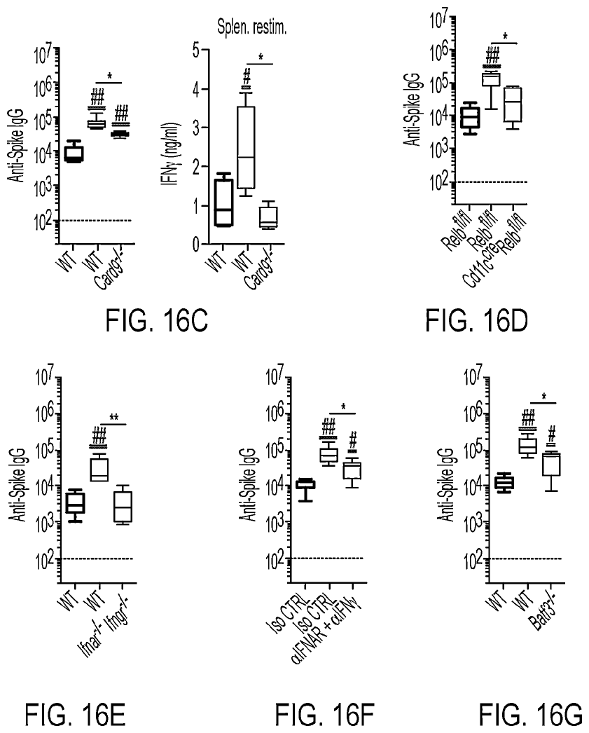

The novel immunological properties observed by formulating mannans with AH prompted us to investigate adjuvant activities in an immunization model of translational relevance. To this end, the pre-fusion stabilized SARS-CoV-2 Spike trimer was used, which has been validated as an antigenic protein that prevents COVID-19 (Corbett et al., 2020; Jackson et al., 2020; Keech et al., 2020; Mercado et al., 2020; Walls et al., 2020; Walsh et al., 2020; Wrapp et al., 2020). Mice were immunized with Spike alone or admixed with AH, β-glucans, mannans or AH/mannans with a prime (day 0)-boost (day 14) schedule. β-glucans were used to control for skin-restricted Dectin activation (FIGS. 1A, 1B and 5B-5D). As soon as 14 days post-prime mannan formulations elicited anti-Spike antibodies (FIG. 6A). On day 28 (14 days post-boost) AH/mannans induced the highest levels of anti-Spike antibodies (FIG. 6A). The same was true for antibodies directed toward the receptor binding domain (RBD) of Spike (FIG. 6B), which is responsible for binding to human ACE2 and SARS-CoV-2 cell entry. Of note, both soluble mannans and AH/mannans promoted anti-Spike type 1 immunity by inducing anti-Spike IgG2c (an antibody subclass induced by IFNγ) and antigen-specific T cells skewed toward IFNγ production (FIGS. 6A-6C), therefore correlating with LN expression of ISGs at early timepoints (FIG. 5D). This evidence is of clinical relevance since antigen-specific type 1 immunity has been associated with reduced risk of vaccine-associated enhanced respiratory disease upon viral infection (Graham, 2020). Notably, elevated levels of anti-Spike IgG1 and IgG2c were maintained up to 98 days post immunization (FIG. 16A). To further assess the protective potential of anti-Spike antibodies elicited in the immunization model, a surrogate virus neutralization test was performed, as well as an actual SARS-CoV-2 neutralization test in which the neutralizing activities of serum samples are respectively measured by the degree of inhibition of recombinant RBD binding to human ACE2 or SARS-CoV-2 cell infection in vitro. In both assays mice immunized with Spike and AH/mannans showed the highest neutralization levels (FIGS. 6D and 6E).

Whether there are spatial and temporal constraints to the adjuvant effect of the AH/mann formulation was also tested by injecting mice with Spike formulated with AH/mann, Spike formulated with AH and mannans injected on the same day or the day before at an adjacent injection site. Interestingly, the enhancement of anti-Spike neutralizing antibodies was only observed when Spike was formulated with AH/mann (FIGS. 6F and 6G). In contrast to B cell responses, IFNγ production by antigen-specific T cells was observed in all conditions in which mice where immunized with AH and mannans, regardless of when and where mannans were injected (FIG. 6H).

Next, the cellular and molecular requirements of the adjuvant effect of AH/mann was investigated. AH/mann-induced anti-Spike IgG response and IFNγ production by antigen-specific T cells were abrogated in Clec4n^{-/-} mice (FIG. 16B) but only partially impaired in Card9^{-/-} mice (FIG. 16C), matching observations of mannan- and AH/mann-induced LIR. In keeping with a key role of the transcription factor RelB in driving mannan-dependent responses, immunization of Cd11c^{cre} Relb^{-/-} mice also showed impairment in the anti-Spike IgG response (FIG. 16D). It was then tested if AH/mann adjuvant effect required type I/II IFN pathways. Either Ifnar^{-/-} Ifngr^{-/-} mice or WT mice in which IFN signaling was transiently abrogated were used during the immunization phase via the administration of anti-IFNAR and anti-IFNγ antibodies. It was observed that both constitutive and acute inhibition of IFN signaling abrogated the effect of the AH/alum formulation (FIGS. 16E and 16F). Finally, AH/mann adjuvant effect required cDC1 as assessed by impaired IgG production in Batf3^{-/-} mice (FIG. 16G).

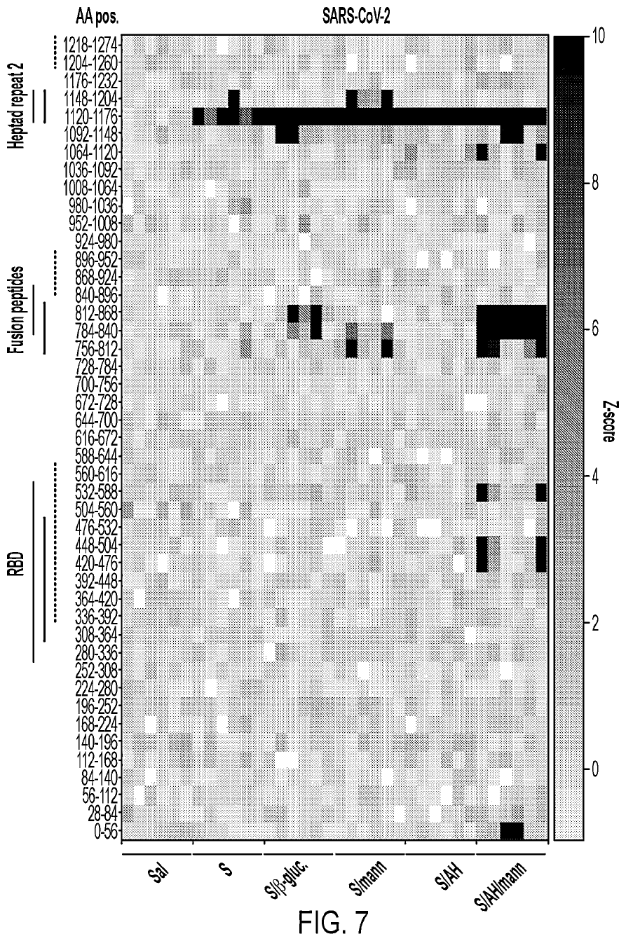

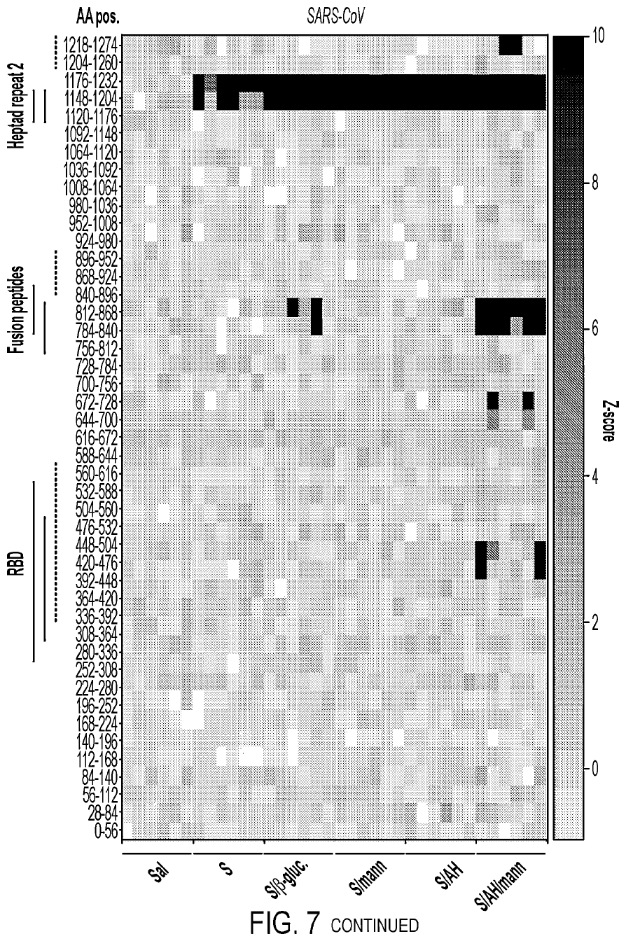

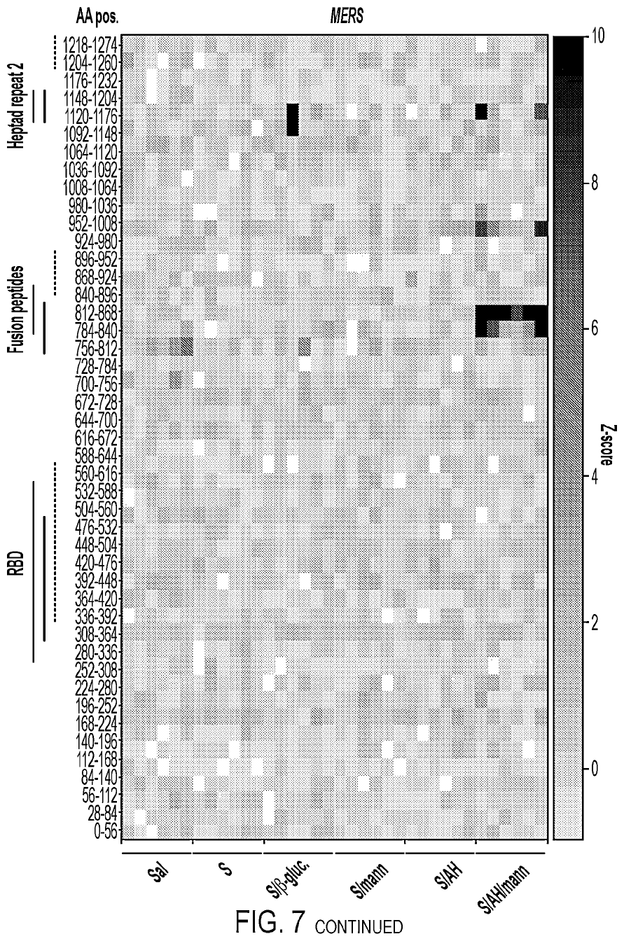

Due to the superior adjuvant activity of the AH/mann formulation, its capacity to induce a broader spectrum of anti-Spike antibodies was tested. To address this point, a VirScan analysis was performed to probe Spike epitopes targeted by antibodies elicited by the different adjuvant formulations (Shrock et al., 2020; Xu et al., 2015a). Interestingly, all formulations induced antibodies against a region that corresponded to the SARS-CoV-2 Heptad repeat 2 while only AH/mannans induced antibodies directed toward the SARS-CoV-2 Fusion peptides and RBD (FIG. 7). This epitope targeting profile was comparable between SARS-CoV-2 and SARS-CoV Spike proteins while fewer epitopes were also targeted on MERS Spike (FIG. 7), which is consistent with Spike protein sequence homology of these three coronaviruses (Hicks et al., 2020). Altogether, the results show that mannan formulations enhance anti-Spike antibody levels and promote anti-Spike type 1 immunity, with AH/mannans being particularly effective at inducing anti-Spike neutralizing antibodies with broad epitope specificity.

### The AH/Mann Formulation Confers Protection Against Lung Viral Infections.

The ability of AH/mann to induce neutralizing antibodies with a broad epitope specificity prompted comparison of the AH/mann formulation with other adjuvants that are currently part of FDA-approved vaccines (O'Hagan et al., 2020). AH/mann was benchmarked against squalene-based oil-in-water nano-emulsions (AS03-like AddaS03 or MF59-like AddaVax) and the AS04-like formulation AH/PHAD prepared by simple admixture of AH and PHAD, a synthetic structural analog of the monophosphoryl lipid A.

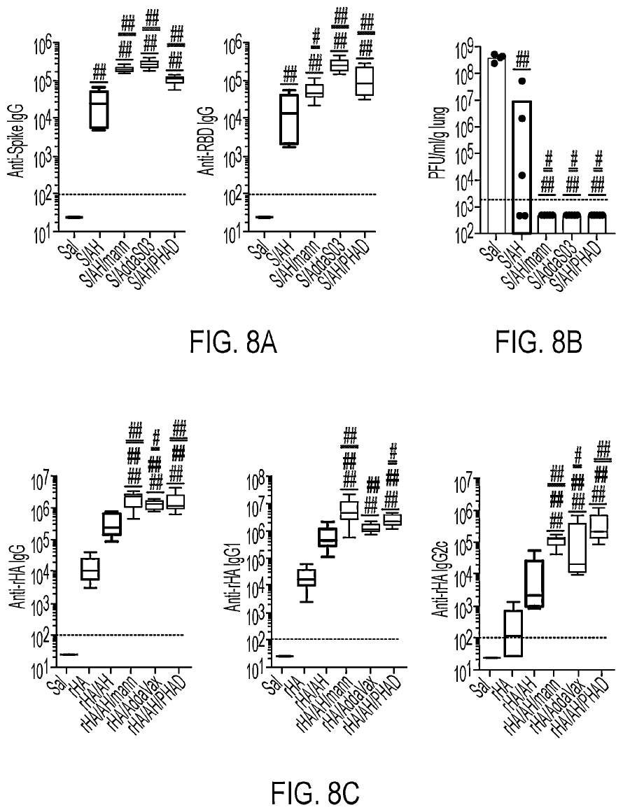

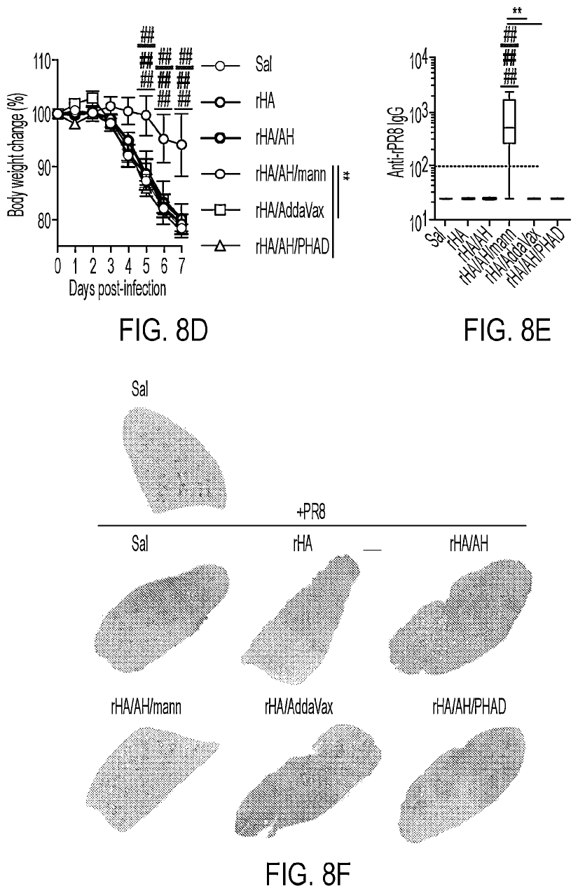

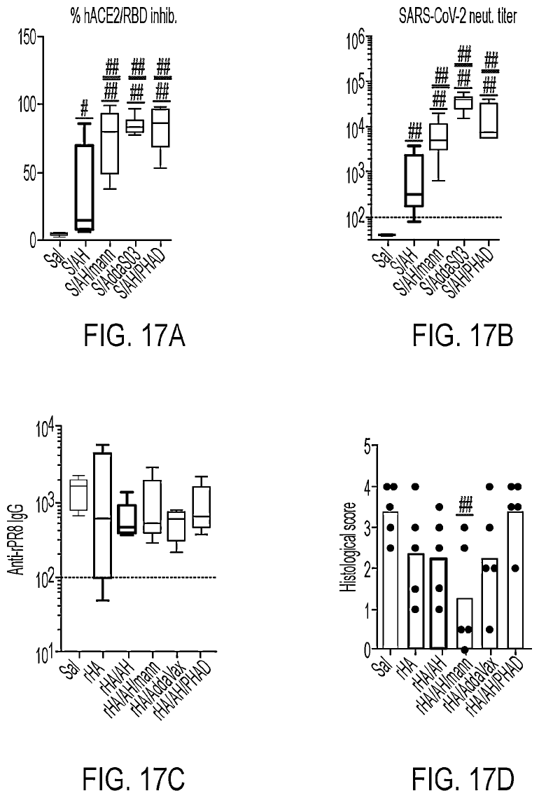

Immunization of mice with Spike formulated with AH/mann, AddaS03 or AH/PHAD elicited comparable levels of anti-Spike and anti-RBD antibodies IgG (FIG. 8A). In keeping with previous results, increased anti-Spike and anti-RBD antibodies correlated with a significant increased neutralization capacity compared to AH alone (FIGS. 17A and 17B). To prove the functional relevance of these antibody responses, immunized mice were infected with the murine adapted SARS-CoV-2 MA10 strain (Leist et al., 2020) and markedly reduced viral lung titers were found in mice immunized with AH/mann, AddaS03 or AH/PHAD compared to saline-treated or AH-immunized mice (FIG. 8B).

These results show that AH/mann enhances both magnitude and breadth of the antigen-specific antibody response. These attributes might be relevant for additional viral glycoproteins characterized by high antigenic variability, such as influenza A virus (IAV) hemagglutinin (HA) and neuraminidase (NA). Based on antigenic diversity and phylogenetic analysis 18 HA and 11 NA proteins have been identified so far, and they combine to form multiple IAV subtypes (e.g., HIN1). Many viral strains have been identified within each subtype, generating high subtypic diversity (Sangesland and Lingwood, 2021). It was therefore reasoned that AH/mann might not only promote a robust antibody response against target antigens but heterosubtypic immunity upon influenza vaccine immunization. To this aim, the clinically relevant recombinant HA (rHA) vaccine Flublok (season 2020-2021, composed of HAs from IAV A/Guangdong-Maonan/SWL1536/2019 [HiN1], IAV A/HongKong/2671/2019 [H3N2], influenza B virus B/Washington/02/2019 and influenza B virus B/Phuket/3073/2013) was employed. Immunized mice with Flublok alone or formulated with AH, AH/mann, Addavax, or AH/PHAD. Anti-rHA antibodies were significantly increased in mice immunized with rHA and AH/mann, AddaVax or AH/PHAD (FIG. 8C). Mice were next challenged intranasally with the IAV strain A/PR/8/1934 which belongs to the HiN1 subtype but whose HA is not part of the Flublok vaccine. Surprisingly, only mice previously immunized with Flublok formulated with AH/mann were significantly protected, as measured by decreased weight loss (FIG. 8D) and cell infiltration in the lungs (FIGS. 8F and 17D). These results correlated with high IgG levels against recombinant HA derived from A/PR/8/1934 (rPR8) in mice immunized with Flublok and AH/mann but not the other adjuvant formulations (FIG. 8E).

Overall, these data demonstrate that AH/mann adjuvant formulation enhances both magnitude and breadth of antibody response against multiple viral glycoproteins in clinically-relevant immunization models.

### DISCUSSION

Activation of innate immune cells by PRR ligands is regarded as a critical step for the initiation of the adaptive immune response (Banchereau and Steinman, 1998; Iwasaki and Medzhitov, 2004; Janeway and Medzhitov, 2002; Matzinger, 1994). The cellular and molecular events triggered by PRR-mediated activation of innate immune cells have been an intense area of investigation, identifying signaling organelles, metabolic pathways and gene expression profiles that shape the innate immune response (Brubaker et al., 2015). However, cell-intrinsic features of PRR activation and signaling are not sufficient to explain the complexity of the in vivo inflammatory response elicited by innate stimuli. Indeed, their localization at cellular and organismal levels plays a key role in determining the activation status of innate immune cells (Evavold and Kagan, 2019). Recent evidence has shown that tuning the physical properties (e.g., solubility and diameter) of PRR ligands, and in particular TLR agonists, has a critical impact on modulating tissues targeting and innate immune activation profiles. Compared to soluble ligands, the conjugation of TLR agonists to polymers that form submicron-sized particles enables more effective traffic to the lymph node and innate immune cell activation. This translates into more potent B and T cell responses in immunization models (Lynn et al., 2015). These results show the importance of defining how modulation of the physical properties of PRR ligands impact location and magnitude of innate immune cell activation. In this regard, the study of CLRs and specifically of Dectin-1 and -2 is of particular interest. These receptors bind the fungal cell wall polysaccharides β-glucans (Dectin-1) and mannans (Dectin-2), and are particularly sensitive to their physical properties since only the particulate form of the fungal polysaccharides induces efficient receptor clustering and activation (Goodridge et al., 2011; Zhu et al., 2013). It was shown that soluble mannans stimulate a potent LIR characterized by LN expansion and expression of type I and II IFN transcriptional responses. Soluble mannans target CD169⁺ sinus macrophages in the dLN and the LIR induced by these fungal ligands was specific to this anatomical location, as these fungal components did not elicit pro-inflammatory responses in vitro and in vivo at the injection site (skin). The physical and immunologic properties of mannans were further modulated by adsorbing them onto aluminum hydroxide (AH) and generating a formulation in which mannans are present in both soluble and particulate (i.e., bound to AH) form at an almost equal ratio. As predicted by its physical properties, this formulation elicits pro-inflammatory responses in vitro and in vivo at both the periphery and the dLN. When tested in an experimental model of immunization with the SARS-CoV-2 Spike protein, both soluble mannans and mannans formulated with AH promote anti-Spike type 1 immunity. However, only the latter elicits anti-Spike neutralizing antibodies with broad epitope specificity that cross-react with SARS-CoV Spike and protect against SARS-CoV-2 MA10 challenge to the same extent as adjuvant formulations that part of FDA-approved vaccines. Notably, immunization of mice with recombinant hemagglutinin (rHA)-based Flublok vaccine formulated with AH and mannans elicited heterosubtypic immunity, as assessed by induction of IgG directed against rHA of an influenza A HIN1 strain (A/PR/8/1934) that was not present in the Flublok vaccine as well as protection against A/PR/8/1934 challenge. Overall, this research sheds light on the molecular pathways activated by mannan formulations that trigger lymph node innate and adaptive responses and result into enhanced magnitude and breadth of antibody responses against viral glycoproteins.

Dectin-1 and -2 have been mainly involved in the development of innate and adaptive immune responses to fungal infection (Borriello et al., 2020; Brown et al., 2018; Lionakis et al., 2017; Netea et al., 2008). There is some evidence that their ligands (e.g., β-glucans and mannans) can be used as vaccine adjuvants. However, a detailed understanding of their mechanism of action is missing (Petrovsky and Cooper, 2011). As previously reported, it was found that β-glucans but not mannans activate innate immune cells in vitro and in vivo at the injection site. Unexpectedly, it was found that both β-glucans and mannans elicit LN expansion, with the latter also inducing unique LN-restricted type I and II IFN signatures. These results can be explained by the physical form of these polysaccharides rather than differences in signaling pathways, since both Dectin-1 and -2 activate the same pathway but mannans are soluble and small in diameter and therefore traffic to the LN while β-glucans are large and insoluble, leading to retention in the skin. To further corroborate this point, the studies demonstrate that a preparation of soluble glucans can also induce LN expansion and ISG expression in the absence of skin inflammation. These results are interesting for several reasons: 1) they establish a relationship between the physical form of fungal polysaccharides and the LN innate immune response; 2) they show feasibility of targeting Dectin receptors to the LN, which is an effective strategy to elicit adaptive immunity (Lian et al., 2020; Liu et al., 2014; Lynn et al., 2015; Scales et al., 2018; Woodruff et al., 2014); 3) early adjuvant-induced type II IFN in the LN is conserved across mammalian species and promotes vaccine immunogenicity (Coccia et al., 2017).

Both type I and II IFNs are required to sustain mannan-induced lymphocyte accrual and LN expansion. It is still unclear whether in this model type I and II IFNs act on the same cell subset or on different ones, a likely occurrence due to the ubiquitous distribution of their receptors. IFNs act on both myeloid cells and lymph node stromal cells modulating a range of functions, including chemokine expression and vascular permeability (Barrat et al., 2019; Ivashkiv, 2018). Hence, the results raise the possibility that mannan-elicited IFN signatures affect LN-resident myeloid and stromal compartments eventually leading to lymphocyte recruitment.

A detailed mechanistic analysis of mannan-induced LIR is provided herein, showing that it requires Dectin-2-expressing, CD169⁺ sinus macrophages, but not cDC1. An unexpected finding is the partial requirement of CARD9, which is a key signaling molecule downstream of Dectin receptors and required for activation of canonical NFkB subunits (Borriello et al., 2020). However, CARD9-independent pathways have also been described including NIK-dependent activation of the non-canonical NFkB subunits p52 and RelB (Gringhuis et al., 2009; Xu et al., 2018). Since mice lacking NIK, p52 or RelB have profound defects in secondary lymphoid organ development (Sun, 2017), mice were employed in which RelB is selectively deleted in CD11c-expressing cells since these cells are required for mannan-induced LIR and to preserve secondary lymphoid organ development. The results show that RelB regulates mannan-induced expression of type I ISGs and cooperate with CARD9 in modulating the expression of type II ISGs. Contrasting evidence on the role of RelB in modulating IFN responses and ISG expression has been generated so far (Le Bon et al., 2006; Saha et al., 2020). It is likely that receptor- and cell-specific differences will play a key role and will need to be taken into account in order to fully understand the role of RelB in mannan-induced ISG expression.

A remarkable feature of the study is that mannans formulated with AH acquire novel immunological properties, namely induction of a pro-inflammatory response in vitro and in vivo at both the injection site and the dLN. Although a synergism between AH and mannans cannot be excluded, these results are more readily explained by concurrent presence in the AH/mannans formulation of unbound and AH-adsorbed mannans. The former is responsible for ISG expression in the dLN while the latter mediates skin inflammation. Evaluation of AH/mann in an immunization model with the pre-fusion stabilized SARS-CoV-2 Spike trimer support this model. Direct synergism between AH and mannan inflammatory activity was excluded when AH and mannans were injected separately but in close proximity, and their capacity to enhance anti-Spike IgG was lost. Intriguingly, this does not apply to T cell-mediated responses that were boosted by the presence of mannans, independently from their coformulation with AH. Analysis of soluble mannans and AH/mannans in an immunization model with the pre-fusion stabilized SARS-CoV-2 Spike trimer revealed that both formulations induce humoral and cellular type 1 immunity, suggesting that early induction of type I and II IFN signatures in the dLN is sufficient to explain polarization of the adaptive immune response. IFNγ can promote both IgG2c antibody switching as well as Th1 polarization (Finkelman et al., 1988; Martin-Fontecha et al., 2004). Notably, transient disruption of type I/II IFN signaling via administration of blocking antibodies significantly decreased anti-Spike IgG, suggesting that early IFN signatures in the dLN translate into long-term potentiation of the immune response.

A unique property of the AH/mannans formulation is also the induction in mice immunized with SARS-CoV-2 Spike of neutralizing anti-Spike antibodies with broad epitope specificity and that cross-react with SARS-CoV Spike and, to a lesser extent, MERS Spike. Production of these antibodies has the same molecular and cellular requirements as the LIR induced by AH/mann, with the only exception of cDC1 that play important roles in driving the IgG response, but not the LIR. Neutralizing antibodies play a key role in protecting against SARS-CoV-2 infection in experimental animal models (Cao et al., 2020; Hassan et al., 2020; Lv et al., 2020; McMahan et al., 2020; Rogers et al., 2020; Schafer et al., 2021; Shi et al., 2020; Tortorici et al., 2020; Zheng et al., 2020; Zost et al., 2020). Accordingly, mice immunized with Spike and AH/mann show undetectable lung viral titers after infection with SARS-CoV-2 MA10 similarly to mice immunized with clinically relevant adjuvant formulations such as AddaS03 and AH/PHAD. In addition, the epitope specificity profile observed in mice immunized with AH/mannans is comparable to the one observed in COVID-19 patients, highlighting the translational relevance of the results (Shrock et al., 2020). To further expand these findings, AH/mann was tested in a model of influenza immunization using the clinically relevant Flublok vaccine composed of recombinant HA (rHA) from two influenza A and two influenza B strains and compared it to MF59-like AddaVax and AS04-like AH/PHAD adjuvant formulations. While all adjuvants enhanced the antibody response to Flublok, only AH/mann induced heterosubtypic immunity, namely protection against challenge with an HIN1 influenza A strain (A/PR/8/1934) whose HA is not contained in the Flublok vaccine. These results were paralleled by detection of antibodies against HA of A/PR/8/1934 only in mice immunized with AH/mann. There are at least two possible non-mutually exclusive explanations for this phenomenon: 1) AH/mannans formulation induces a high degree of innate immune activation by concurrently targeting the periphery and dLN, thereby enhancing adaptive immunity; 2) AH generates a depot that slowly releases antigen to the dLN and/or AH promotes the formation of antigen multimers, that together with the LIR induced by unbound mannans can promote the germinal center reaction (Moyer et al., 2020; Pedersen et al., 2020). It will be important in the future to clarify how AH/mannans enhance antigen-specific adaptive immunity and modulate germinal center dynamics and B cell repertoire selection. To that end, while the in vivo experiments described herein utilized an efficacious formulation of AH/mannans in a 1:5 molar ratio, subsequent experiments suggest that a formulation of 2:5 AH/mannans further enhances in vitro cytokine expression from bone marrow derived phagocytes, thereby highlighting the importance of AH for potentiating the immunogenicity of mannans. From a translational perspective, the broadening of epitope specificity suggests that AH/mann formulation coupled with appropriate antigens might be a promising candidate for the development vaccines targeting multiple coronavirus or influenza A strains.

Overall, the study provides mechanistic understanding of different modalities of Dectin-2 targeting enabled by modulation of the physical form of mannans, their translational impact for the development of more effective vaccines, and further extend knowledge of the relationship between the physical form of innate stimuli and LN innate and adaptive immune responses.

### Methods

Mice: C57BL/6J (Jax 00664) (wild type), CB6F1 (Jax 100007), B6.129P2(C)-Ccr7^{tm1Rfor}/J (Ccr7^{-/-}, Jax 006621), B6.129S2-Ifnar1^{tm1Agt}/Mmjax (Ifnar^{-/-}, Jax 32045-JAX), B6.Cg-Ifngr1^{tm1Agt} Ifnar1^{tm1.2Ees}/J (Ifnar^{-/-} Ifngr^{-/-}, Jax 029098), B6.FVB-1700016L21Rik^{Tg(Itgax-DTR/EGFP)57Lan}/J (CD11c-DTR, Jax 004509), B6.Cg-Tg(Itgax-cre)1-1Reiz/J (Cd11c^{cre}, Jax 008068), B6.Cg-Relb^{tm1Ukl}/J (Relb^{fl/fl}, Jax 028719), B6.129S4-Ccr2^{tm1Ifc}/J (Ccr2^{-/-}, Jax 004999), B6.129-Card9^{tm1Xlin}X/J (Card9^{-/-}, Jax 028652) and B6.129S6-Clec7a^{tm1Gdb}/J (Clec7a^{-/-}, Jax 012337) were purchased from Jackson Labs. B6.129P2-Fcer1g^{tm1Rav} N12 (Fcerlg^{-/-}, Model 583) were purchased from Taconic. Clec4n^{-/-} mice were kindly provided by Drs. Nora A. Barrett and Yoichiro Iwakura. B6; 129-Siglec1<tm1(HBEGF)Mtka>(CD169-DTR) mice were kindly provided by Dr. F. Pucci and are from the Riken Institute (No. RBRC04395), deposited by Drs. Kenji Kohno and Masato Tanaka (Miyake et al., 2007; Saito et al., 2001). B6.Cg-Tg(TcraTcrb)425Cbn/J (OT-II, Jax 004194) were kindly provided by Juan Manuel Leyva-Castillo. Female mice were used for all the experiments. Mice were housed under specific pathogen-free conditions at Boston Children's Hospital, and all the procedures were approved under the Institutional Animal Care and Use Committee (IACUC) and operated under the supervision of the department of Animal Resources at Children's Hospital (ARCH).
Reagents and antibodies: for flow cytometry, imaging cytometry, fluorescence-activated cell sorting (FACS) and confocal microscopy experiments the following reagents and antibodies were used: anti-CD45 BV510 (30-F11), anti-CD45 Alexa Fluor 700 (30-F11), anti-CD45 APC (30-F11), anti-CD3 PE/Dazzle 594 (17A2), anti-CD3 BV510 (17A2), anti-CD19 PE/Dazzle 594 (6D5), anti-CD19 BV650 (6D5), anti-NK1.1 PE/Dazzle 594 (PK136), anti-Ter119 PE/Dazzle 594 (TER-119), anti-I-A/I-E PE/Cy7 (M5/114.15.2), anti-Ly6G PerCP/Cy5.5 (1A8), anti-CD11b Pacific Blue (M1/70), anti-Ly6C BV711 (HK1.4), anti-CD11c BV785 (N418), anti-CD11c APC (N418), anti-CD86 APC/Cy7 (GL-1), anti-CD86 APC (GL-1), anti-OX40L PE (RM134L), anti-CD169 APC (3D6.112), anti-CD169 Alexa Fluor 647 (3D6.112), anti-CD4 APC/Fire 750 (GK1.5), anti-CD45R/B220 Alexa Fluor 594 (RA3-6B2), anti-CD3 Biotin (145-2C11), anti-CD19 Biotin (6D5), anti-NK1.1 Biotin (PK136), anti-Ter119 Biotin (TER-119), TrueStain FcX (93), True-Stain Monocyte Blocker and Zombie Red Fixable Viability Kit were purchased from Biolegend; anti-Dectin-2 PE (REA1001) was purchased from Miltenyi Biotec; rat anti-Dectin-2 (D2.11E4) was purchased from GeneTex; anti-phopsho-Syk (Tyr525/526) (C87C1) was purchased from Cell Signaling Technology; CellTrace CFSE Cell Proliferation Kit, Alexa Fluor 488 NHS Ester (Succinimidyl Ester) and DAPI were purchased from Thermo Fisher Scientific.

For in vitro and in vivo experiments the following reagents were used: Iscove's Modified Dubecco's Medium (IMDM), Phosphate Buffer Saline (PBS), penicillin/streptomycin (pen/strep) and L-Glutamine (L-Gln) were purchased from Lonza; Fetal Bovine Serum (FBS) was purchased from Thermo Fisher Scientific; collagenase from Clostridium histolyticum, deoxyribonuclease (DNase) I from bovine pancreas and dispase II were purchased from MilliporeSigma; TLRGrade Escherichia coli LPS (Serotype O555:B5, 1 μg/ml) was purchased from Enzo Life Sciences; curdlan (10 μg/ml) was purchased from Wako Chemicals; mannans, β-glucans and their Alexa Fluor 488-conjugates (10 μg/ml for in vitro experiments, 500 μg/mouse for in vivo experiments) were provided by Michael D Kruppa, Zuchao Ma and David L Williams (East Tennessee State University); carboxyl latex beads 3 μm were purchased from Thermo Fisher Scientific and used directly (cell:bead ratio 1:10 for in vitro experiments) or after coating with diaminopropane derivatized mannans provided by Michael D Kruppa, Zuchao Ma and David L Williams (East Tennessee State University); WGP-S and WGP-D (500 μg/mouse for in vivo experiments) were purchased from Invivogen; Lipo-CpG was provided by Darrell J. Irvine (Koch Institute for Integrative Cancer Research at MIT); diphtheria toxin (unnicked) from Corynebacterium diphtheriae (200 ng/mouse for CD11-DTR mice, 500 ng/mouse for CD169-DTR mice) was purchased form Cayman Chemical; ovalbumin (OVA) EndoFit (5 μg/mouse) and Alhydrogel adjuvant 2% (aluminum hydroxide, 2 μg/ml for in vitro experiments, 100 μg/mouse for in vivo experiments) were purchased from Invivogen; recombinant pre-fusion stabilized SARS-CoV-2 Spike trimer (1 μg/mouse) and RBD were expressed and purified from plasmids generously provided by Drs. Berney S. Graham (NIH Vaccine Research Center) and Aaron G. Schmidt (Ragon Institute), respectively; SARS-CoV-2 Spike peptide pools (PepTivator SARS-CoV-2 Prot_S) were purchased from Miltenyi Biotec. anti-CD62L (Mel-14, 100 μg/mouse), anti-IFNγ (XMG1.2, 200 μg/mouse), anti-Ly6G (1A8, 50 μg/mouse) and their isotype controls rat IgG2a (2A3) and rat IgG1 (HRPN) were purchased from Bio X Cell.

The AH/mannan (AH/mann) formulation was obtained by admixture of AH (100 μg/10 μl), mannans (500 μg/25 μl) and saline (15 μl). When formulated with an antigen (e.g., SARS-CoV-2 Spike trimer or 2020 season Flublok) the volume of saline was reduced accordingly in order to keep the total volume constant. This formulation is further described in the Vaccine Adjuvant Compendium (https://vac.niaid.nih.gov).

2020 season Flublok recombinant hemaglutinin (rHA) is commercially available from Sanofi Pasteur and contains rHA from the following influenza virus strains in equal molar ratio: influenza A/Guangdong-Maonan/SWL1536/2019 [HiN1], influenza A/HongKong/2671/2019 [H3N2], influenza B virus B/Washington/02/2019, and influenza B virus B/Phuket/3073/2013.

Isolation of mannan from C. albicans. Candida albicans strain SC5314 was maintained on blood agar (Remel) plates grown at 37° C. For mannan isolation, C. albicans was inoculated into 15 l of YPD (1% yeast extract, 2% peptone, 2% dextrose) and grown for 20 hours at 37° C. Cells were harvested by centrifugation at 5000 g for 5 minutes. This resulted in a 100 g pellet from 15 l of media. A standard protocol was used for isolation and NMR characterization of the mannan (Kruppa et al., 2011; Lowman et al., 2011). In brief, the cell pellets were suspended in 200 ml of acetone to delipidate the cells for 20 minutes prior to centrifugation at 5000 g for 5 minutes, removal of acetone and drying of the pellet for an hour. Dried pellets were broken up and transferred to a beadbeater. An equivalent volume of acid-washed glass beads was added and 200 ml of dH₂O was added to the mixture. The cells were subjected to bead beating for three 30 second pulses before the entire mixture was transferred to a 1 l flask. The material was autoclaved for 2 hours, allowed to cool and then centrifuged for 5 minutes at 5000 g. The supernatant was retained and the cell pellet discarded. Pronase (500 mg in 20 ml dH₂O), which had been filter sterilized and heat treated for 20 minutes at 65° C. (to remove any glycosidic activity) was added to the supernatant along with sodium azide to a concentration of 1 mM. The mixture was then incubated overnight (20 hours) at 37° C. to allow for degradation of any proteins in the solution. Mannans were extracted by addition of an equal volume of Fehling's solution to the protease treated mannan solution and allowed to mix for one hour at room temperature. After mixing the solution was allowed to stand for 20 minutes to facilitate mannan precipitation. The supernatant was decanted and the precipitate was dissolved in 10 ml of 3M HCl, to enable release of copper from the reducing ends of the mannans. To the dissolved mannan solution 500 ml of an 8:1 mixture of methanol:acetic acid was added, and the mixture stirred to allow the mannan to precipitate overnight. After the material had settled, the supernatant was decanted, washed again with 500 ml of methanol, allowing six hours for the mannans to settle. The supernatant was decanted and the remaining precipitate was dissolved in 200 ml dH₂O. The mannans were dialyzed against a 200-fold change of dH₂O over 48 hours using a 2000 MW cutoff membrane to remove residual acid, methanol and other compounds from the extraction process. The dialysate was then subjected to lyophilization and stored at −20° C. until needed. A small sample (10 mg) of the material was subjected to NMR to confirm for the purity of the N-linked mannans (Lowman et al., 2011) and for assessment of molecular weight (Kruppa et al., 2011). Prior to in vitro or in vivo use the mannan is depyrogenated to remove any residual endotoxin and filter sterilized.
Preparation of the diaminopropane (DAP) derivatized mannan. Mannan (100 mg) was dissolved in 1 ml of dimethyl sulfoxide (DMSO) in 4 ml vial after one hour of stirring. 1,3-Diaminopropane (100 μL) was added and stirred at ambient temperature for 3 hours. Sodium cyanoborohydride (100 mg) was added and the reaction mixture was stirred for 48 hours, followed by addition of sodium borohydride (50 mg) and stirring for 24 hours. Acetic acid (200.0 μl) was added dropwise at 0° C. to quench the reaction and the reaction mixture was stirred at ambient temperature for 3 hours, then dialyzed with a 1000 MWCO RC membrane against ultrapure water (1000 ml×4). The retentate was harvested and lyophilized to yield the DAP attached mannan. The recovery was 88.5 mg, ˜88%. The mannan-DAP was characterized by 1H-NMR to confirm the identity of the compound.

For conjugation with Alexa Fluor 488 NHS Ester (Succinimidyl Ester), ˜15 mg of mannan-DAP were resuspended in 1 ml of sodium borate conjugation buffer (100 mM, pH 8.5) and allowed to solvate for at least 24 hours. Then, 1 mg of Alexa Fluor 488 NHS Ester resuspended in 35 μl of DMSO was added to the solution and incubated overnight in the dark at room temperature with gentle agitation. The reaction mixture was dialyzed with a 6000-8000 MWCO RC membrane against saline (1000 ml×4) and the retentate was filter sterilized. For conjugation with carboxyl latex beads 3 μm, mannan-DAP was resuspended at a concentration of 10 mg/1 ml of BupH MES conjugation buffer pH 4.5 (Thermo Fisher Scientific) and allowed to solvate for at least 24 hours. 1 ml of mannan-DAP was added to 50×10⁶ beads and then mixed with 4 mg/1 ml of EDC (Thermo Fisher Scientific) resuspended in pure water. The reaction mixture was incubated for 4 hours in the dark at room temperature with gentle agitation. Then, the beads were washed twice (4000 g for 10 minutes) with saline and resuspended in saline at a concentration of 10⁸ beads/ml.

Preparation of Candida albicans β-glucan particles. β-glucan particles were isolated from Candida albicans SC5314 as previously described by the laboratory (Lowman et al., 2014). Briefly, glucan was isolated from C. albicans using a base/acid extraction approach with provides water insoluble glucan particles that are ≥95% pure. The structure and purity of the glucan was determined by 1H-NMR in DMSO-d6 (Lowman et al., 2014). Prior to in vitro or in vivo use the β-glucan particles are depyrogenated and filter sterilized.
Preparation of the diaminopropane (DAP) derivatized β-glucan. β-glucan particles (20 mg) were dissolved in 1 ml of dimethyl sulfoxide (DMSO) in 4 ml vial after one hour of stirring. 1,3-Diaminopropane (100 μL) was added and stirred at ambient temperature for 3 hours. Sodium cyanoborohydride (100 mg) was added and the reaction mixture was stirred for 48 hours, followed by addition of sodium borohydride (50 mg) and stirring for 24 hours. Acetic acid (200 μl) was added dropwise at 0° C. to quench the reaction and the reaction mixture was stirred at ambient temperature for 3 hours. The β-glucan particles were harvested and washed five times in water by centrifugation (862 g). The recovery was >95%. The glucan-DAP was characterized by ¹H-NMR to confirm the identity of that the structure of the mannan was not changed and also to detect the presence of the DAP. The glucan-DAP was lyophilized to dryness and stored at −20° C. in the dark in a desiccator until needed.

For conjugation with Alexa Fluor 488 NHS Ester (Succinimidyl Ester), 20 mg of glucan-DAP were suspended in 1 ml of sodium borate conjugation buffer (100 mM, pH 8.5) and allowed to hydrate for at least 24 hours at 4° C. Then, 1 mg of Alexa Fluor 488 NHS Ester resuspended in 35 μl of DMSO was added to the solution and incubated overnight in the dark at room temperature with gentle agitation. The reaction mixture was centrifuged, washed five times (862 g) and the 488 labeled glucan particles were harvested.

Quantification of mannans in the aluminum hydroxide/mannans formulation. Supernatants were harvested from the AH/mannans mixture. The supernatants were lyophilized. The lyophilized supernatants and a mannan that was not mixed with AH (4.0 mg) were dissolved in 500 μl of deuterium oxide (99.9% D) with 0.01% (W/V) internal standard TMSP-2,2,3,3-D₄ (98.0% D). ¹H NMR data were collected on a Bruker Avance 400 MHz Ultra Shield NMR Spectrometer at 295 K with the same acquisition parameters for all the samples. NMR spectra were processed using TOPSPIN 2.1 running on the Avance 400 MHz NMR. The ring proton resonances (3.25-4.50 ppm) were integrated referencing to the integral of internal standard (−0.02-0.02 ppm) calibrated as 1. Based on the ratio between standard mannan mass (4.0 mg) and its ring proton integral (avg. 39.12), the mannan masses in the supernatants were calculated using the detected ring proton integrals adjusted for the blanks. The amount of mannan absorbed by the AH was determined by the relative mass losses of mannan in the supernatants after formulation.
Analysis of skin and LN responses. To assess skin and LN innate responses, mice were intradermally injected on day 0 with the indicated compounds in a volume of 50 μl on each side of the back (one side for the compound and the contralateral side for saline of vehicle control). 6 or 24 hours post-injection skin samples at the injection sites and draining (brachial) LNs were collected for subsequent analysis.

Skin samples were transferred to a beadbeater and homogenized in 1 ml of TRI Reagent (Zymo Research). Then, samples were centrifuged 12000 g for 10 minutes and 800 μl of cleared supernatant were transferred to a new tube for subsequent RNA isolation. LNs were weighted on an analytical scale before being transferred to a beadbeater and homogenized in TRI Reagent as indicated for skin samples or processed to generate a LN cell suspension by modification of a previously published protocol (Fletcher et al., 2011). Briefly, individual LNs were incubated at 37° C. for 20 minutes in 400 μl of digestion mix (IMDM+pen/strep+FBS 2%+collagenase 100 mg/ml+dispase II 100 mg/ml+DNase 10 mg/ml). Then, LNs were grinded by pipetting with a 1000 μl tip, supernatants were transferred to new tubes and kept at 4° C. while 200 μl of digestion mix were added to the pellets and incubated at 37° C. for 10 minutes. This cycle was repeated one more time, then pooled supernatants of individual LNs were divided into two aliquots: one for flow cytometry analysis, another one was centrifuged at 300 g for 5 minutes and the cell pellet was resuspended in 800 μl of TRI Reagent for subsequent RNA isolation.

For specific experiments mice were treated with: anti-CD62L blocking antibody or isotype control, intravenous injections on day −1; anti-IFNγ blocking antibody or isotype control, intravenous injections on day −1 and 0; anti-Ly6G depleting antibody or isotype control, intraperitoneal injections on day −1 and 0; diphtheria toxin, intravenous injections on day −1 and intradermal injections (co-injected with mannans) on day 0 for CD11c-DTR mice, intraperitoneal injection on day −2 for CD169-DTR mice.

In vitro stimulation of GM-CSF-differentiated, bone marrow-derived phagocytes. Bone marrow-derived phagocytes were differentiated from bone marrow in IMDM+10% B16-GM-CSF derived supernatant+10% FBS+pen/strep+L-Gln and used after 7 days of culture. Then, cells were harvested, plated in flat bottom 96 well plates at a density of 10⁵ cells/200 μl/well in IMDM+10% FBS+pen/strep+L-Gln and stimulated with the indicated compounds for 18-21 hours. At the end of stimulation, supernatants were harvested and TNF and IL-2 concentrations were measured by ELISA (Biolegend) according to the manufacturer's protocol. Cells were detached with PBS+EDTA 2 mM and transferred to a round bottom 96 well plate for subsequent flow cytometry staining and analysis. Alternatively, cells were stimulated for 6 hours, lysed in TRI Reagent and RNA was extracted for gene expression analysis.
In vivo quantification of fluorescently labelled β-glucans and mannans. Mice were intradermally injected with 500 μg/mouse of Alexa Fluor 488-conjugated β-glucans and mannans (in selected experiments mannans were formulated with 100 μg/mouse of AH before injection). At the indicated timepoints dLNs were collected, transferred to a beadbeater and homogenized in 400 μl of deionized water. Then, samples were centrifuged (12000 g for 10 minutes) and cleared supernatants were transferred to a 96 well clear bottom black plate. Fluorescence values were measured with SpectraMax i3×microplate reader (Molecular Devices) and expressed as arbitrary units after background (deionized water) subtraction.
Flow cytometry, fluorescence-activated cell sorting (FACS), imaging cytometry and confocal microscopy. For flow cytometry analysis, cells were first stained with Zombie Red Fixable Viability in PBS for 5 minutes at 4° C., washed once with PBS+BSA 0.2%+NaN₃ 0.05% (300 g for 5 minutes) and then stained with antibodies against surface antigens diluted in PBS+BSA 0.2%+NaN₃ 0.05% for 20 minutes at 4° C. Cells were then washed, fixed with 2% paraformaldehyde for 10 minutes at room temperature, washed again and resuspended in PBS+BSA 0.2%+NaN₃ 0.05%. Samples were acquired on a BD LSRFortessa flow cytometer and data were analyzed using FlowJo v.10 software (BD Biosciences). CountBright Absolute Counting Beads were used to quantify absolute cell numbers. In selected experiments, after fixation with 2% paraformaldehyde cells were permeabilized by incubation with a saponin-based permeabilization buffer (BioLegend) for 10 minutes at 4° C. and stained with antibodies against intracellular cytokines diluted in permeabilization buffer for 20 minutes at 4° C. Then, cells were washed with permeabilization buffer, resuspended in PBS+BSA 0.2%+NaN3 0.05% and acquired as indicated before.

For FACS and imaging cytometry, mice were intradermally injected with AF488-mannans and 6 hours later dLNs were harvested to obtain LN cell suspensions. For FACS, cells were stained with antibodies against surface antigens diluted in PBS+BSA 0.2% for 20 minutes at 4° C. Cells were then washed once, resuspended in 1 ml of PBS+BSA 0.2%, filtered through 70 μm cell strainers (Fisher Scientific) and sorted with a Sony MA900 cell sorter directly into 1 ml of TRI Reagent. The following cell subset was sorted: CD3⁻ CD19⁻ NK1.1⁻ Ter119⁻ CD45⁺ AF488-mannan⁺ Ly6G⁻ (CD11b⁺ Ly6C⁺)⁻ CD11b⁺ CD11c⁺. For imaging cytometry, cells were depleted of lymphoid and erythroid cells by sequential staining with biotinylated antibodies against anti-CD3, anti-CD19, anti-NK1.1, anti-Ter119 and Streptavidin Microbeads (Miltenyi Biotec) according to the manufacturer's protocol. The remaining cells were stained with anti-CD45 APC, fixed with 2% paraformaldehyde, washed once and resuspended in 60 μl of PBS+DAPI (0.2 μg/ml). Samples were then acquired on an Amnis ImageStream X Mark II (Luminex Corporation). Mannan internalization was analyzed with Amnis Ideas Software and calculated with Internalization Feature as AF488 signal within the APC mask.

For confocal microscopy, dLNs were isolated at steady state or 1 hour post-injection of AF488-mannans and fixed with 4% paraformaldehyde overnight. Tissue slides were prepared from frozen LN samples at the Beth Israel Deaconess Medical Center (BIDMC) Histology Core Facility and stained at the BIDMC Confocal Imaging Core Facility. Briefly, frozen sections were air-dried for 30 minutes and rehydrated. The sections were permeabilized using 0.05% Triton X-100 for 10 minutes at room temperature and washed three times with TBS. The sections were then incubated with 5% normal donkey serum (Jackson ImmunoResearch Lab) for 1 hour at room temperature. For Dectin-2 staining of steady state LNs, slides were incubated with rat anti-Dectin-2 overnight at 4° C. The slides were washed three times and incubated with: Alexa Fluor 488-conjugated Donkey anti-rat secondary antibody (Jackson ImmunoResearch Lab) for 90 minutes at room temperature and washed four times. Slides were then incubated with Alexa Fluor 647-conjugated rat anti-CD169 primary antibody and Alexa Fluor 594-conjugated rat anti-CD45R/B220 primary antibody for 90 minutes at room temperature and then washed with TBS. For phospho-Syk staining of AF488-mannan-treated LNs, slides were incubated with rabbit anti-phospho-Syk (Cell Signaling Technology) overnight at 4° C. The slides were washed three times and incubated with Alexa Fluor 647-conjugated Donkey anti-rabbit secondary antibody (Jackson ImmunoResearch Lab) for 90 minutes at room temperature and washed four times. Slides were then incubated with Alexa Fluor 594-conjugated rat anti-CD45R/B220 primary antibody for 90 minutes at room temperature and then washed with TBS. rabbit anti-phospho-Syk (Cell Signaling Technology). Samples were counterstained with Hoechst 33342 (Thermo Fisher Scientific) and washed three times with TBS. Slides were mounted with Prolong Gold anti-fade mounting media (Thermo Fisher Scientific) and imaged on a Zeiss 880 laser scanning confocal microscope at the Boston Children's Hospital Harvard Digestive Disease Center.

RNA isolation, qPCR, transcriptomic and pathway analyses. RNA was isolated from TRI Reagent samples using phenol-chloroform extraction or column-based extraction systems (Direct-zol RNA Microprep and Miniprep, Zymo Research) according to the manufacturer's protocol. RNA concentration and purity (260/280 and 260/230 ratios) were measured by NanoDrop (Thermo Fisher Scientific).

Purified RNA was analyzed for gene expression by qPCR on a CFX384 real time cycler (Bio-rad) using pre-designed KiCqStart SYBR Green Primers (MilliporeSigma) specific for Cxc19 (RM1_Cxcl9 and FM1_Cxcl9), Gbp2 (RM1_Gpb2 and FM1_Gbp2), Ifit2 (RM1_Ifit2 and FM1_Ifit2), Rsad2 (RM1_Rsad2 and FM1_Rsad2), Il6 (RM1_Il6 and FM1_Il6), Cxcl1 (RM1_Cxcl1 and FM1_Cxcl1) or pre-designed PrimeTime qPCR Primers (Integrated DNA Technologies) specific for Gapdh (Mm.PT.39a.1).

For bulk RNAseq analysis, RNA isolated from LN samples was submitted to Genewiz. RNA samples were quantified using Qubit 2.0 Fluorometer (Thermo Fisher Scientific) and RNA integrity was checked with RNA Screen Tape on Agilent 2200 TapeStation (Agilent Technologies). RNA sequencing library preparation was prepared using TruSeq Stranded mRNA library Prep kit following manufacturer's protocol (Illumina, Cat #RS-122-2101). Briefly, mRNAs were first enriched with Oligod(T) beads. Enriched mRNAs were fragmented for 8 minutes at 94° C. First strand and second strand cDNA were subsequently synthesized. The second strand of cDNA was marked by incorporating dUTP during the synthesis. cDNA fragments were adenylated at 3′ends, and indexed adapter was ligated to cDNA fragments. Limited cycle PCR was used for library enrichment. The incorporated dUTP in second strand cDNA quenched the amplification of second strand, which helped to preserve the strand specificity. Sequencing libraries were validated using DNA Analysis Screen Tape on the Agilent 2200 TapeStation (Agilent Technologies), and quantified by using Qubit 2.0 Fluorometer (Thermo Fisher Scientific) as well as by quantitative PCR (Applied Biosystems). The sequencing libraries were multiplexed and clustered on 1 lane of flowcell. After clustering, the flowcell was loaded on the Illumina HiSeq instrument according to manufacturer's instructions. The samples were sequenced using a 2×150 Pair-End (PE) High Output configuration. Image analysis and base calling were conducted by the HiSeq Control Software (HCS) on the HiSeq instrument. Raw sequence data (.bcl files) generated from Illumina HiSeq was converted into fastq files and de-multiplexed using Illumina bcl2fastq program version 2.17. One mismatch was allowed for index sequence identification. Reads were quality-controlled using FastQC. Illumina adapters were removed using cutadapt. Trimmed reads were mapped to the mouse transcriptome (GRCm38) based on Ensembl annotations using Kallisto (Bray et al., 2016). Transcript counts were imported and aggregated to gene counts using tximport (Soneson et al., 2015). Gene counts were analyzed using the R package DESeq2 (Love et al., 2014). When applicable, batch was used as a blocking factor in the statistical model. Differentially expressed genes (DEGs) were identified as those passing a threshold of FDR significance threshold (0.05 for skin; 0.01 for lymph nodes, a more stringent threshold thanks to the greater power due to higher number of replicates) where the alternate hypothesis was that the absolute log 2 FC was greater than 0. Genes induced by mannan or glucan treatment over saline were plotted in heatmaps using the R package ComplexHeatmap, using Z-scored log 2 normalized abundance. Genes were arranged by abundance delta between glucan and mannan (aggregated from multiple time points when appropriate), with a gap delimiting two clusters: genes more highly expressed upon mannan stimulation vs genes more highly expressed upon glucan stimulation. Pathway analysis was performed with the R package hypeR (Federico and Monti, 2020), using hypergeometric enrichment tests of genes belonging to a cluster of interest and the Hallmark gene set collection from the Broad Institute's MSigDB collection.

For targeted transcriptome sequencing, 25 ng of RNA isolated from sorted cells was retrotranscribed to cDNA using SuperScript VILO cDNA Synthesis Kit (Thermo Fisher Scientific). Barcoded libraries were prepared using the Ion AmpliSeq Transcriptome Mouse Gene Expression Kit as per the manufacturer's protocol and sequenced using an Ion S5 system (Thermo Fisher Scientific). Differential gene expression analysis was performed using the ampliSeqRNA plugin (Thermo Fisher Scientific). To quantify the number of DEGs, gene-level fold change<−1.5 or >1.5 and gene-level p value<0.05 (ANOVA) were considered. For heatmap representation, DEGs were defined with an F-test FDR less than 0.05 and a log 2 fold-change (FC) greater than 1 (or lower than −1) between a mutant and WT control. Hierarchical clustering was performed with Pearson correlation and average linkage. Pathway analysis was performed with the R package hypeR, using Kolmogorov Smirnov Test on genes ranked according to their log 2FC.

In vivo CD4⁺ and CD8⁺ T cell proliferation assay. Spleens were isolated from OT-II or OT-I mice and meshed with the plunger end of a syringe. Then, splenocyte cell suspensions were treated with ACK lysis buffer (2 ml for 2 minutes at room temperature), washed with PBS (300 g for 5 minutes) and filtered through 70 mm cell strainers. CD4⁺ and CD8⁺ T cells were respectively purified using CD4 (L3T4) or CD8a (Ly-2) MicroBeads (Miltenyi Biotec) according to the manufacturer's protocol and stained with CellTrace CFSE (5 mM in PBS+FBS 2.5% for 20 minutes in the dark). At the end of incubation, cells were washed twice with PBS, resuspended at a concentration of 5×10⁵ cells/100 ml saline and 100 ml of cell suspension was intravenously injected into each mouse. 24 hours later (day 0) mice were intradermally injected with OVA (5 mg/mouse) alone or combined with mannans (500 mg/mouse). Saline-injected mice were used as control. On day +3 dLNs were harvested and LN cell suspension were stained with anti-CD19, anti-Ter119, anti-CD3 and anti-CD4 or anti-CD8 antibodies. Adoptively transferred, CFSE-labelled OT-II and OT-I cells were respectively detected in the CD19⁻ Ter119⁻ CD3⁺ CD4⁺ and CD19⁻ Ter119⁻ CD3⁺ CD8⁺ gates. Results are expressed as absolute number of CD19⁻ Ter119⁻ CD3⁺ CD4⁺ CFSE^{lo} or CD19⁻ Ter119⁻ CD3⁺ CD8⁺ CFSE^{lo} cells (i.e., cells undergoing at least one division cycle) or percentage of each division peak within the CD19⁻ Ter119⁻ CD3⁺ CD4⁺ or CD19⁻ Ter119⁻ CD3⁺ CD8⁺ gates.
SARS-CoV-2 Spike and RBD expression and purification. Full length SARS-CoV-2 spike glycoprotein and RBD constructs (amino acid residues R319-K529), both with an HRV3C protease cleavage site, a TwinStrepTag and an 8× HisTag at C-terminus, were obtained from Drs. Barney S. Graham (NIH Vaccine Research Center) and Aaron G. Schmidt (Ragon Institute), respectively. These expression vectors were used to transiently transfect Expi293 cells (Thermo Fisher Scientific) using polyethylenimine (Polysciences). Protein was purified from filtered cell supernatants using either StrepTactin resin (IBA) or Cobalt-TALON resin (Takara). Affinity tags were cleaved off from eluted protein samples by HRV3C protease and tag removed proteins were subjected to additional purification by size-exclusion chromatography using either a Superose 6 10/300 column (GE Healthcare) or a Superdex 75 10/300 Increase column (GE Healthcare) in PBS (pH 7.4) buffer.
Immunization and antibody quantification. CB6F1 mice were immunized by intradermal injection of Spike (1 mg/mouse) alone or formulated with AH (100 mg/mouse), β-glucans (500 mg/mouse), mannans (500 mg/mouse), AH/mannans (AH/mann), AddaS03 or AH/PHAD on day 0 and day +14. Alternatively, C57BL/6 mice were immunized by intradermal injection Flublok vaccine (1 μg Flubok 2020; 0.25 μg per rHA) alone or formulated with AH (100 μg AH), AH/mann (100 μg AH, 500 μg mannans), AddaVax or AH/PHAD on day 0 and day +14. Saline-injected mice were used as control. Blood samples were collected by retroorbital bleeding on day +14 (pre-boost) and day +28, and serum samples were isolated after centrifugation of blood samples twice at 1500 g for 10 minutes. In selected experiments blood samples were collected on day +98 or 7 days post-challenge. Spike-, RBD-, Flublok- and rPR8-specific IgG, IgG1, IgG2c antibody levels were quantified in serum samples by ELISA by modification of a previously described protocol (Borriello et al., 2017). Briefly, high binding flat bottom 96-well plates were coated with 0.5 μg/ml Spike, 1 μg/ml RBD, 1 μg/ml Flublok or 1 μg/ml rPR8 in PBS, incubated overnight at 4° C., washed once with PBS+0.05% Tween-20 (PBST) and blocked with PBS+BSA 1% for 1 hour at room temperature. Then, serum samples were added with an initial dilution of 1:100 and 1:4 serial dilutions in PBS+BSA 1% to generate 11-point curves and incubated for 2 hours at room temperature. Plates were then washed three times with PBST and incubated for 1 hour at room temperature with HRP-conjugated anti-mouse IgG, IgG1 or IgG2c (Southern Biotech) antibodies. At the end of the incubation, plates were washed five times with PBST and developed with tetramethylbenzidine (BD OptEIA Substrate Solution for Spike, rHA, and rPR8; Thermo Fisher Scientific 1-Step Ultra TMB-ELISA Substrate Solution for RBD) for 5 min, then stopped with 2 N H₂SO₄. Optical densities (ODs) were read at 450 nm with SpectraMax iD3× microplate reader (Molecular Devices) and endpoint titers were calculated using as cutoff three times the optical density of the background. Values<100 were reported as 25.
Splenocyte restimulation assay. Immunized mice were sacrificed on day 35 and their spleens were collected. To isolate splenocytes, spleens were mashed through a 70 μm cell strainer and the resulting cell suspensions were washed with PBS and incubated with ACK lysis buffer (2 ml for 2 minutes at room temperature) to lyse erythrocytes. Splenocytes were washed again with PBS and plated in flat bottom 96-well plates (2×10⁶ cells per well). Then, SARS-CoV-2 Spike peptides (PepTivator SARS-CoV-2 Prot_S, Miltenyi Biotec) were added at a final concentration of 0.6 nmol/ml (total cell culture volume, 200 μl per well). After 96 hours, supernatants were harvested and IFNγ levels were measured by ELISA (Thermo Fisher Scientific) according to the manufacturer's protocol.
SARS-CoV-2 surrogate virus neutralization tests. The surrogate virus neutralization test was performed by modification of a previously published protocol (Tan et al., 2020). Briefly, high binding flat bottom 96-well plates were coated with 2 μg/ml recombinant human ACE2 (hACE2, MilliporeSigma) in PBS, incubated overnight at 4° C., washed three times with PBST and blocked with PBS+BSA 1% for 1 hour at room temperature. In the meantime, each serum sample (final dilution 1:160) was pre-incubated with 3 ng of RBD-Fc (R&D Systems) in PBS+BSA 1% for 1 hour at room temperature and then transferred to the hACE2-coated plate. As positive control, RBD-Fc was also added to hACE2-coated wells without pre-incubation with serum samples. After 1 hour at room temperature, plates were washed three times with PBST and incubated with an HRP-conjugated anti-human IgG Fc antibody (Southern Biotech) for 1 hour at room temperature. At the end of the incubation, plates were washed five times with PBST and developed with tetramethylbenzidine (BD Biosciences) for 5 min, then stopped with 2 N H₂SO₄. The optical density was read at 450 nm with SpectraMax iD3× microplate reader (Molecular Devices). Percentage inhibition of RBD binding to hACE2 was calculated with the following formula: Inhibition (%)=[1−(Sample OD value−Background OD value)/(Control OD value−Background OD value)]×100.
SARS-CoV-2 Neutralization Titer Determination. All serum samples were heat inactivated at 56° C. for 30 minutes to remove complement and allowed to equilibrate to room temperature prior to processing for neutralization titer. Samples were diluted in duplicate to an initial dilution of 1:5 or 1:10 followed by 1:2 serial dilutions, resulting in a 12-dilution series with each well containing 100 μl. All dilutions were performed in DMEM (Quality Biological), supplemented with 10% (v/v) fetal bovine serum (heat inactivated, MilliporeSigma), 1% (v/v) penicillin/streptomycin (Gemini Bio-products) and 1% (v/v) L-glutamine (2 mM final concentration, Thermo Fisher Scientific). Dilution plates were then transported into the BSL-3 laboratory and 100 μl of diluted SARS-CoV-2 (WA-1, courtesy of Dr. Natalie Thornburg/CDC) inoculum was added to each well to result in a multiplicity of infection (MOI) of 0.01 upon transfer to titering plates. A non-treated, virus-only control and a mock infection control were included on every plate. The sample/virus mixture was then incubated at 37° C. (5.0% CO₂) for 1 hour before transferring to 96-well titer plates with confluent VeroE6 cells. Titer plates were incubated at 37° C. (5.0% CO₂) for 72 hours, followed by cytopathic effect (CPE) determination for each well in the plate. The first sample dilution to show CPE was reported as the minimum sample dilution required to neutralize>99% of the concentration of SARS-CoV-2 tested (neut99).
VirScan. Phage IP and sequencing was performed as described previously (Xu et al., 2015a) with slight modifications. A sublibrary encoding a 56-mer peptide library tiling every 28 amino acids through the proteomes of the six HCoVs and three bat coronaviruses most closely related to SARS-CoV-2 (Shrock et al., 2020) was mixed with the original VirScan library to enable mapping of SARS-CoV-2 epitopes. 0.6 μl mouse sera, or approximately 2 μg of IgG, was included in each VirScan reaction. Immunoprecipitations were performed using magnetic protein A and protein G Dynabeads (Thermo Fisher Scientific) as previously described (Xu et al., 2015a).
Statistics. When necessary, data were Log-transformed before statistical analysis to approximate normal distributions. One-sample t test was used to compare each group against the value 1 (or 0 after Log-transformation, which represent the contralateral control sample expressed as fold). Statistical differences between groups in datasets with one categorical variable were evaluated by two sample t test (2 groups) or one-way ANOVA (more than 2 groups) corrected for multiple comparisons. Statistical differences between groups in datasets with two categorical variables were evaluated by two-way ANOVA corrected for multiple comparisons. #or * and ** or ##respectively indicate p≤0.05 and 0.01.

### REFERENCES

Acton, S. E., Farrugia, A. J., Astarita, J. L., Mourao-Sa, D., Jenkins, R. P., Nye, E., Hooper, S., van Blijswijk, J., Rogers, N. C., Snelgrove, K. J., et al. (2014). Dendritic cells control fibroblastic reticular network tension and lymph node expansion. Nature 514, 498-502.
Acton, S. E., and Reis e Sousa, C. (2016). Dendritic cells in remodeling of lymph nodes during immune responses. Immunol Rev 271, 221-229.
Bachmann, M. F., and Jennings, G. T. (2010). Vaccine delivery: a matter of size, geometry, kinetics and molecular patterns. Nat Rev Immunol 10, 787-796.
Banchereau, J., and Steinman, R. M. (1998). Dendritic cells and the control of immunity. Nature 392, 245-252.
Barrat, F. J., Crow, M. K., and Ivashkiv, L. B. (2019). Interferon target-gene expression and epigenomic signatures in health and disease. Nature immunology 20, 1574-1583.
Borriello, F., Pietrasanta, C., Lai, J. C. Y., Walsh, L. M., Sharma, P., O'Driscoll, D. N., Ramirez, J., Brightman, S., Pugni, L., Mosca, F., et al. (2017). Identification and Characterization of Stimulator of Interferon Genes As a Robust Adjuvant Target for Early Life Immunization. Front Immunol 8, 1772.
Borriello, F., Zanoni, I., and Granucci, F. (2020). Cellular and molecular mechanisms of antifungal innate immunity at epithelial barriers: The role of C-type lectin receptors. Eur J Immunol.
Bray, N. L., Pimentel, H., Melsted, P., and Pachter, L. (2016). Near-optimal probabilistic RNA-seq quantification. Nat Biotechnol 34, 525-527.
Brown, G. D., Willment, J. A., and Whitehead, L. (2018). C-type lectins in immunity and homeostasis. Nat Rev Immunol 18, 374-389.
Brubaker, S. W., Bonham, K. S., Zanoni, I., and Kagan, J. C. (2015). Innate immune pattern recognition: a cell biological perspective. Annu Rev Immunol 33, 257-290.
Cancel, J. C., Crozat, K., Dalod, M., and Mattiuz, R. (2019). Are Conventional Type 1 Dendritic Cells Critical for Protective Antitumor Immunity and How? Front Immunol 10, 9.
Cao, Y., Su, B., Guo, X., Sun, W., Deng, Y., Bao, L., Zhu, Q., Zhang, X., Zheng, Y., Geng, C., et al. (2020). Potent Neutralizing Antibodies against SARS-CoV-2 Identified by High-Throughput Single-Cell Sequencing of Convalescent Patients' B Cells. Cell 182, 73-84 e16.
Coccia, M., Collignon, C., Herve, C., Chalon, A., Welsby, I., Detienne, S., van Helden, M. J., Dutta, S., Genito, C. J., Waters, N. C., et al. (2017). Cellular and molecular synergy in AS01-adjuvanted vaccines results in an early IFNgamma response promoting vaccine immunogenicity. NPJ Vaccines 2, 25.
Corbett, K. S., Flynn, B., Foulds, K. E., Francica, J. R., Boyoglu-Barnum, S., Werner, A. P., Flach, B., O'Connell, S., Bock, K. W., Minai, M., et al. (2020). Evaluation of the mRNA-1273 Vaccine against SARS-CoV-2 in Nonhuman Primates. N Engl J Med 383, 1544-1555.
De Giovanni, M., Cutillo, V., Giladi, A., Sala, E., Maganuco, C. G., Medaglia, C., Di Lucia, P., Bono, E., Cristofani, C., Consolo, E., et al. (2020). Spatiotemporal regulation of type I interferon expression determines the antiviral polarization of CD4(+) T cells. Nature immunology 21, 321-330.
Didierlaurent, A. M., Collignon, C., Bourguignon, P., Wouters, S., Fierens, K., Fochesato, M., Dendouga, N., Langlet, C., Malissen, B., Lambrecht, B. N., et al. (2014). Enhancement of adaptive immunity by the human vaccine adjuvant AS01 depends on activated dendritic cells. J Immunol 193, 1920-1930.
Eisenbarth, S. C., Colegio, O. R., O'Connor, W., Sutterwala, F. S., and Flavell, R. A. (2008). Crucial role for the Nalp3 inflammasome in the immunostimulatory properties of aluminium adjuvants. Nature 453, 1122-1126.
Evavold, C. L., and Kagan, J. C. (2019). Inflammasomes: Threat-Assessment Organelles of the Innate Immune System. Immunity 51, 609-624.
Federico, A., and Monti, S. (2020). hypeR: an R package for geneset enrichment workflows. Bioinformatics 36, 1307-1308.
Finkelman, F. D., Katona, I. M., Mosmann, T. R., and Coffman, R. L. (1988). IFN-gamma regulates the isotypes of Ig secreted during in vivo humoral immune responses. J Immunol 140, 1022-1027.
Fletcher, A. L., Malhotra, D., Acton, S. E., Lukacs-Kornek, V., Bellemare-Pelletier, A., Curry, M., Armant, M., and Turley, S. J. (2011). Reproducible isolation of lymph node stromal cells reveals site-dependent differences in fibroblastic reticular cells. Front Immunol 2, 35.
Garcia-Vello, P., Speciale, I., Chiodo, F., Molinaro, A., and De Castro, C. (2020). Carbohydrate-based adjuvants. Drug Discov Today Technol 35-36, 57-68.
Goodridge, H. S., Reyes, C. N., Becker, C. A., Katsumoto, T. R., Ma, J., Wolf, A. J., Bose, N., Chan, A. S., Magee, A. S., Danielson, M. E., et al. (2011). Activation of the innate immune receptor Dectin-1 upon formation of a ‘phagocytic synapse’. Nature 472, 471-475.
Graham, B. S. (2020). Rapid COVID-19 vaccine development. Science 368, 945-946.
Grant, S. M., Lou, M., Yao, L., Germain, R. N., and Radtke, A. J. (2020). The lymph node at a glance—how spatial organization optimizes the immune response. J Cell Sci 133.
Gringhuis, S. I., den Dunnen, J., Litjens, M., van der Vlist, M., Wevers, B., Bruijns, S. C., and Geijtenbeek, T. B. (2009). Dectin-1 directs T helper cell differentiation by controlling noncanonical NF-kappaB activation through Raf-1 and Syk. Nature immunology 10, 203-213.
Hassan, A. O., Case, J. B., Winkler, E. S., Thackray, L. B., Kafai, N. M., Bailey, A. L., McCune, B. T., Fox, J. M., Chen, R. E., Alsoussi, W. B., et al. (2020). A SARS-CoV-2 Infection Model in Mice Demonstrates Protection by Neutralizing Antibodies. Cell 182, 744-753 e744.
Hicks, J., Klumpp-Thomas, C., Kalish, H., Shunmugavel, A., Mehalko, J., Denson, J. P., Snead, K. R., Drew, M., Corbett, K. S., Graham, B. S., et al. (2021). Serologic Cross-Reactivity of SARS-CoV-2 with Endemic and Seasonal Betacoronaviruses. J Clin Immunol 41, 906-913.
Irvine, D. J., Aung, A., and Silva, M. (2020). Controlling timing and location in vaccines. Adv Drug Deliv Rev 158, 91-115.
Ivashkiv, L. B. (2018). IFNgamma: signalling, epigenetics and roles in immunity, metabolism, disease and cancer immunotherapy. Nat Rev Immunol 18, 545-558.
Iwasaki, A., and Medzhitov, R. (2004). Toll-like receptor control of the adaptive immune responses. Nature immunology 5, 987-995.
Jackson, L. A., Anderson, E. J., Rouphael, N. G., Roberts, P. C., Makhene, M., Coler, R. N., McCullough, M. P., Chappell, J. D., Denison, M. R., Stevens, L. J., et al. (2020). An mRNA Vaccine against SARS-CoV-2—Preliminary Report. N Engl J Med 383, 1920-1931.
Janeway, C. A., Jr., and Medzhitov, R. (2002). Innate immune recognition. Annu Rev Immunol 20, 197-216.
Kastenmuller, W., Torabi-Parizi, P., Subramanian, N., Lammermann, T., and Germain, R. N. (2012). A spatially-organized multicellular innate immune response in lymph nodes limits systemic pathogen spread. Cell 150, 1235-1248.
Keech, C., Albert, G., Cho, I., Robertson, A., Reed, P., Neal, S., Plested, J. S., Zhu, M., Cloney-Clark, S., Zhou, H., et al. (2020). Phase 1-2 Trial of a SARS-CoV-2 Recombinant Spike Protein Nanoparticle Vaccine. N Engl J Med 383, 2320-2332.
Kruppa, M., Greene, R. R., Noss, I., Lowman, D. W., and Williams, D. L. (2011). C. albicans increases cell wall mannoprotein, but not mannan, in response to blood, serum and cultivation at physiological temperature. Glycobiology 21, 1173-1180.
Le Bon, A., Montoya, M., Edwards, M. J., Thompson, C., Burke, S. A., Ashton, M., Lo, D., Tough, D. F., and Borrow, P. (2006). A role for the transcription factor RelB in IFN-alpha production and in IFN-alpha-stimulated cross-priming. Eur J Immunol 36, 2085-2093.
Leal, J. M., Huang, J. Y., Kohli, K., Stoltzfus, C., Lyons-Cohen, M. R., Olin, B. E., Gale, M., Jr., and Gerner, M. Y. (2021). Innate cell microenvironments in lymph nodes shape the generation of T cell responses during type I inflammation. Sci Immunol 6.
Leist, S. R., Dinnon, K. H., 3rd, Schafer, A., Tse, L. V., Okuda, K., Hou, Y. J., West, A., Edwards, C. E., Sanders, W., Fritch, E. J., et al. (2020). A Mouse-Adapted SARS-CoV-2 Induces Acute Lung Injury and Mortality in Standard Laboratory Mice. Cell 183, 1070-1085 e1012.
Lian, J., Ozga, A. J., Sokol, C. L., and Luster, A. D. (2020). Targeting Lymph Node Niches Enhances Type 1 Immune Responses to Immunization. Cell Rep 31, 107679.
Lionakis, M. S., Iliev, I. D., and Hohl, T. M. (2017). Immunity against fungi. JCI Insight 2.
Liu, H., Moynihan, K. D., Zheng, Y., Szeto, G. L., Li, A. V., Huang, B., Van Egeren, D. S., Park, C., and Irvine, D. J. (2014). Structure-based programming of lymph-node targeting in molecular vaccines. Nature 507, 519-522.
Love, M. I., Huber, W., and Anders, S. (2014). Moderated estimation of fold change and dispersion for RNA-seq data with DESeq2. Genome Biol 15, 550.
Lowman, D. W., Ensley, H. E., Greene, R. R., Knagge, K. J., Williams, D. L., and Kruppa, M. D. (2011). Mannan structural complexity is decreased when Candida albicans is cultivated in blood or serum at physiological temperature. Carbohydr Res 346, 2752-2759.
Lowman, D. W., Greene, R. R., Bearden, D. W., Kruppa, M. D., Pottier, M., Monteiro, M. A., Soldatov, D. V., Ensley, H. E., Cheng, S. C., Netea, M. G., et al. (2014). Novel structural features in Candida albicans hyphal glucan provide a basis for differential innate immune recognition of hyphae versus yeast. J Biol Chem 289, 3432-3443.
Lv, Z., Deng, Y. Q., Ye, Q., Cao, L., Sun, C. Y., Fan, C., Huang, W., Sun, S., Sun, Y., Zhu, L., et al. (2020). Structural basis for neutralization of SARS-CoV-2 and SARS-CoV by a potent therapeutic antibody. Science 369, 1505-1509.
Lynn, G. M., Laga, R., Darrah, P. A., Ishizuka, A. S., Balaci, A. J., Dulcey, A. E., Pechar, M., Pola, R., Gemer, M. Y., Yamamoto, A., et al. (2015). In vivo characterization of the physicochemical properties of polymer-linked TLR agonists that enhance vaccine immunogenicity. Nat Biotechnol 33, 1201-1210.
Martin-Fontecha, A., Thomsen, L. L., Brett, S., Gerard, C., Lipp, M., Lanzavecchia, A., and Sallusto, F. (2004). Induced recruitment of NK cells to lymph nodes provides IFN-gamma for T(H)1 priming. Nature immunology 5, 1260-1265.
Matzinger, P. (1994). Tolerance, danger, and the extended family. Annu Rev Immunol 12, 991-1045.
McMahan, K., Yu, J., Mercado, N. B., Loos, C., Tostanoski, L. H., Chandrashekar, A., Liu, J., Peter, L., Atyeo, C., Zhu, A., et al. (2020). Correlates of protection against SARS-CoV-2 in rhesus macaques. Nature.
Mercado, N. B., Zahn, R., Wegmann, F., Loos, C., Chandrashekar, A., Yu, J., Liu, J., Peter, L., McMahan, K., Tostanoski, L. H., et al. (2020). Single-shot Ad26 vaccine protects against SARS-CoV-2 in rhesus macaques. Nature 586, 583-588.
Miyake, Y., Asano, K., Kaise, H., Uemura, M., Nakayama, M., and Tanaka, M. (2007). Critical role of macrophages in the marginal zone in the suppression of immune responses to apoptotic cell-associated antigens. J Clin Invest 117, 2268-2278.
Moran, I., Grootveld, A. K., Nguyen, A., and Phan, T. G. (2019). Subcapsular Sinus Macrophages: The Seat of Innate and Adaptive Memory in Murine Lymph Nodes. Trends Immunol 40, 35-48.
Morefield, G. L., Jiang, D., Romero-Mendez, I. Z., Geahlen, R. L., Hogenesch, H., and Hem, S. L. (2005). Effect of phosphorylation of ovalbumin on adsorption by aluminum-containing adjuvants and elution upon exposure to interstitial fluid. Vaccine 23, 1502-1506.
Moyer, T. J., Kato, Y., Abraham, W., Chang, J. Y. H., Kulp, D. W., Watson, N., Turner, H. L., Menis, S., Abbott, R. K., Bhiman, J. N., et al. (2020). Engineered immunogen binding to alum adjuvant enhances humoral immunity. Nat Med 26, 430-440.
Netea, M. G., Brown, G. D., Kullberg, B. J., and Gow, N. A. (2008). An integrated model of the recognition of Candida albicans by the innate immune system. Nat Rev Microbiol 6, 67-78.
O'Hagan, D. T., Lodaya, R. N., and Lofano, G. (2020). The continued advance of vaccine adjuvants—‘we can work it out’. Semin Immunol 50, 101426.
Ohl, L., Mohaupt, M., Czeloth, N., Hintzen, G., Kiafard, Z., Zwirner, J., Blankenstein, T., Henning, G., and Forster, R. (2004). CCR7 governs skin dendritic cell migration under inflammatory and steady-state conditions. Immunity 21, 279-288.
Pedersen, G. K., Andersen, P., and Christensen, D. (2018). Immunocorrelates of CAF family adjuvants. Semin Immunol 39, 4-13.
Pedersen, G. K., Worzner, K., Andersen, P., and Christensen, D. (2020). Vaccine Adjuvants Differentially Affect Kinetics of Antibody and Germinal Center Responses. Front Immunol 11, 579761.
Petrovsky, N., and Cooper, P. D. (2011). Carbohydrate-based immune adjuvants. Expert Rev Vaccines 10, 523-537.
Rogers, T. F., Zhao, F., Huang, D., Beutler, N., Burns, A., He, W. T., Limbo, O., Smith, C., Song, G., Woehl, J., et al. (2020). Isolation of potent SARS-CoV-2 neutralizing antibodies and protection from disease in a small animal model. Science 369, 956-963.
Saha, I., Jaiswal, H., Mishra, R., Nel, H. J., Schreuder, J., Kaushik, M., Singh Chauhan, K., Singh Rawat, B., Thomas, R., Naik, S., et al. (2020). RelB suppresses type I Interferon signaling in dendritic cells. Cell Immunol 349, 104043.
Saito, M., Iwawaki, T., Taya, C., Yonekawa, H., Noda, M., Inui, Y., Mekada, E., Kimata, Y., Tsuru, A., and Kohno, K. (2001). Diphtheria toxin receptor-mediated conditional and targeted cell ablation in transgenic mice. Nat Biotechnol 19, 746-750.
Sangesland, M., and Lingwood, D. (2021). Antibody Focusing to Conserved Sites of Vulnerability: The Immunological Pathways for ‘Universal’ Influenza Vaccines. Vaccines (Basel) 9.
Santus, W., Barresi, S., Mingozzi, F., Broggi, A., Orlandi, I., Stamerra, G., Vai, M., Martorana, A. M., Polissi, A., Kohler, J. R., et al. (2017). Skin infections are eliminated by cooperation of the fibrinolytic and innate immune systems. Sci Immunol 2.
Scales, H. E., Meehan, G. R., Hayes, A. J., Benson, R. A., Watson, E., Walters, A., Tomura, M., Maraskovsky, E., Garside, P., Baz Morelli, A., et al. (2018). A Novel Cellular Pathway of Antigen Presentation and CD4 T Cell Activation in vivo. Front Immunol 9, 2684.
Schafer, A., Muecksch, F., Lorenzi, J. C. C., Leist, S. R., Cipolla, M., Bournazos, S., Schmidt, F., Maison, R. M., Gazumyan, A., Martinez, D. R., et al. (2021). Antibody potency, effector function, and combinations in protection and therapy for SARS-CoV-2 infection in vivo. J Exp Med 218.
Shi, R., Shan, C., Duan, X., Chen, Z., Liu, P., Song, J., Song, T., Bi, X., Han, C., Wu, L., et al. (2020). A human neutralizing antibody targets the receptor-binding site of SARS-CoV-2. Nature 584, 120-124.
Shrock, E., Fujimura, E., Kula, T., Timms, R. T., Lee, I. H., Leng, Y., Robinson, M. L., Sie, B. M., Li, M. Z., Chen, Y., et al. (2020). Viral epitope profiling of COVID-19 patients reveals cross-reactivity and correlates of severity. Science 370.
Soderberg, K. A., Payne, G. W., Sato, A., Medzhitov, R., Segal, S. S., and Iwasaki, A. (2005). Innate control of adaptive immunity via remodeling of lymph node feed arteriole. Proc Natl Acad Sci USA 102, 16315-16320.
Soneson, C., Love, M. I., and Robinson, M. D. (2015). Differential analyses for RNA-seq: transcript-level estimates improve gene-level inferences. F1000Res 4, 1521.
Sun, S. C. (2017). The non-canonical NF-kappaB pathway in immunity and inflammation. Nat Rev Immunol 17, 545-558.
Tan, C. W., Chia, W. N., Qin, X., Liu, P., Chen, M. I., Tiu, C., Hu, Z., Chen, V. C., Young, B. E., Sia, W. R., et al. (2020). A SARS-CoV-2 surrogate virus neutralization test based on antibody-mediated blockage of ACE2-spike protein-protein interaction. Nat Biotechnol 38, 1073-1078.
Taylor, P. R., Reid, D. M., Heinsbroek, S. E., Brown, G. D., Gordon, S., and Wong, S. Y. (2005). Dectin-2 is predominantly myeloid restricted and exhibits unique activation-dependent expression on maturing inflammatory monocytes elicited in vivo. Eur J Immunol 35, 2163-2174.
Tortorici, M. A., Beltramello, M., Lempp, F. A., Pinto, D., Dang, H. V., Rosen, L. E., McCallum, M., Bowen, J., Minola, A., Jaconi, S., et al. (2020). Ultrapotent human antibodies protect against SARS-CoV-2 challenge via multiple mechanisms. Science 370, 950-957.
Walls, A. C., Park, Y. J., Tortorici, M. A., Wall, A., McGuire, A. T., and Veesler, D. (2020). Structure, Function, and Antigenicity of the SARS-CoV-2 Spike Glycoprotein. Cell 181, 281-292 e286.
Walsh, E. E., Frenck, R. W., Jr., Falsey, A. R., Kitchin, N., Absalon, J., Gurtman, A., Lockhart, S., Neuzil, K., Mulligan, M. J., Bailey, R., et al. (2020). Safety and Immunogenicity of Two RNA-Based Covid-19 Vaccine Candidates. N Engl J Med 383, 2439-2450.
Wong, E., Montoya, B., Stotesbury, C., Ferez, M., Xu, R. H., and Sigal, L. J. (2019). Langerhans Cells Orchestrate the Protective Antiviral Innate Immune Response in the Lymph Node. Cell Rep 29, 3047-3059 e3043.
Wong, E., Xu, R. H., Rubio, D., Lev, A., Stotesbury, C., Fang, M., and Sigal, L. J. (2018). Migratory Dendritic Cells, Group 1 Innate Lymphoid Cells, and Inflammatory Monocytes Collaborate to Recruit NK Cells to the Virus-Infected Lymph Node. Cell Rep 24, 142-154.
Woodruff, M. C., Heesters, B. A., Herndon, C. N., Groom, J. R., Thomas, P. G., Luster, A. D., Turley, S. J., and Carroll, M. C. (2014). Trans-nodal migration of resident dendritic cells into medullary interfollicular regions initiates immunity to influenza vaccine. J Exp Med 211, 1611-1621.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263.
Xu, G. J., Kula, T., Xu, Q., Li, M. Z., Vernon, S. D., Ndung'u, T., Ruxrungtham, K., Sanchez, J., Brander, C., Chung, R. T., et al. (2015a). Viral immunology. Comprehensive serological profiling of human populations using a synthetic human virome. Science 348, aaa0698.
Xu, R. H., Wong, E. B., Rubio, D., Roscoe, F., Ma, X., Nair, S., Remakus, S., Schwendener, R., John, S., Shlomchik, M., et al. (2015b). Sequential Activation of Two Pathogen-Sensing Pathways Required for Type I Interferon Expression and Resistance to an Acute DNA Virus Infection. Immunity 43, 1148-1159.
Xu, X., Xu, J. F., Zheng, G., Lu, H. W., Duan, J. L., Rui, W., Guan, J. H., Cheng, L. Q., Yang, D. D., Wang, M. C., et al. (2018). CARD9(S12N) facilitates the production of IL-5 by alveolar macrophages for the induction of type 2 immune responses. Nature immunology 19, 547-560.
Zhu, L. L., Zhao, X. Q., Jiang, C., You, Y., Chen, X. P., Jiang, Y. Y., Jia, X. M., and Lin, X. (2013). C-type lectin receptors Dectin-3 and Dectin-2 form a heterodimeric pattern-recognition receptor for host defense against fungal infection. Immunity 39, 324-334.
Zost, S. J., Gilchuk, P., Case, J. B., Binshtein, E., Chen, R. E., Nkolola, J. P., Schafer, A., Reidy, J. X., Trivette, A., Nargi, R. S., et al. (2020). Potently neutralizing and protective human antibodies against SARS-CoV-2. Nature 584, 443-449.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of inducing an immune response to a virus in a subject in need thereof, the method comprising administering to the subject a viral antigen and an adjuvantation system comprising a fungal polysaccharide.

2. The method of claim 1, wherein the fungal polysaccharide is a soluble polysaccharide.

3. The method of claim 1 or claim 2, wherein the fungal polysaccharide is a mannan.

4. The method of any of claims 1-3, wherein the fungal polysaccharide is isolated from Candida albicans.

5. The method of any one of claims 1-4, wherein the adjuvantation system further comprises alum.

6. The method of claim 5, wherein fungal polysaccharide is adsorbed into the alum.

7. The method of any one of claims 1-6, wherein the virus is a Beta coronavirus selected from Middle East Respiratory Syndrome coronavirus (MERS-CoV), Severe Acute Respiratory Syndrome (SARS)-associated coronavirus (SARS-CoV)-1, and SARS-CoV-2.

8. The method of any one of claims 1-7, wherein the viral antigen comprises a Beta coronavirus protein or polypeptide.

9. The method of any one of claims 1-7, wherein the viral antigen comprises a nucleic acid encoding a Beta coronavirus protein or a polypeptide.

10. The method of claim 9, wherein the nucleic acid is DNA or RNA.

11. The method of claim 10, wherein the RNA is a messenger RNA (mRNA).

12. The method of any one of claims 8-11, wherein the Beta coronavirus protein or polypeptide comprises a Beta coronavirus spike protein or spike protein receptor binding domain (RBD).

13. The method of claim 12, wherein the Beta coronavirus spike protein is a MERS-CoV spike protein, SARS-CoV-1 spike protein, or SARS-CoV-2 spike protein.

14. The method of claim 12, wherein the Beta coronavirus spike protein RBD is a MERS-CoV spike protein RBD, SARS-CoV-1 spike protein RBD, or SARS-CoV-2 spike protein RBD.

15. The method of any one of claims 1-7, wherein the viral antigen comprises a viral particle of MERS-CoV, SARS-CoV-1, or SARS-CoV-2.

16. The method of any one of claims 1-7, wherein the viral antigen comprises killed or inactivated MERS-CoV, SARS-CoV-1, or SARS-CoV-2.

17. The method of any one of claims 1-7, wherein the viral antigen comprises killed or live attenuated MERS-CoV, SARS-CoV-1, or SARS-CoV-2.

18. The method of any one of claims 1-6, wherein the virus is an influenza A virus or an influenza B virus.

19. The method of any one of claims 1-6 or claim 18, wherein the viral antigen comprises an influenza A virus or influenza B virus protein or polypeptide.

20. The method of any one of claims 1-6 or claim 18, wherein the viral antigen comprises a nucleic acid encoding an influenza A virus or influenza B virus protein or polypeptide.

21. The method of claim 20, wherein the nucleic acid is DNA or RNA.

22. The method of claim 21, wherein the RNA is a mRNA.

23. The method of any one of claims 19-22, wherein the influenza A virus or influenza B virus protein or polypeptide is a hemagglutinin (HA) protein, a neuraminidase (NA) protein, or polypeptide thereof.

24. The method of any one of claims 1-6 or claim 18, wherein the antigen comprises a viral particle of an influenza A virus or an influenza B virus.

25. The method of any one of claims 1-6 or claim 18, wherein the antigen comprises killed or inactivated influenza A virus or influenza B virus.

26. The method of any one of claims 1-6 or claim 18, wherein the antigen comprises killed or live attenuated influenza A virus or influenza B virus.

27. The method of any one of claims 1-26, wherein the subject is human.

28. The method of claim 27, wherein the subject is a human neonate, a human infant, an adult human, or an elderly human.

29. The method of any one of claims 1-26, wherein the subject is a companion animal or a research animal.

30. The method of any one of claims 1-29, wherein the subject is immune-compromised, has chronic lung disease, asthma, cardiovascular disease, cancer, obesity, diabetes, chronic kidney disease, and/or liver disease.

31. The method of any one of claims 1-30, wherein the viral antigen and the adjuvantation system are administered simultaneously.

32. The method of any one of claims 1-30, wherein the viral antigen and the adjuvantation system are administered separately.

33. The method of any one of claims 1-32, wherein the viral antigen and the adjuvantation system are administered intramuscularly, intradermally, orally, intravenously, topically, intranasally, or sublingually.

34. The method of any one of claims 1-33, wherein the administration is prophylactic.

35. The method of any one of claims 1-34, wherein the administration elicits a type 1 immune response in the subject.

36. The method of any one of claims 1-35, wherein the adjuvantation system promotes the activation of dendritic cell-associated C-type lectin 2 (Dectin-2) in the subject

37. The method of any one of claims 1-36, wherein the adjuvantation system leads to an innate immune response of the subject.

38. The method of any one of claims 1-37, wherein the adjuvantation system enhances B cell immunity.

39. The method of any one of claims 1-38, wherein the adjuvantation system enhances the production of antigen-specific antibodies, compared to when the viral antigen is administered alone.

40. The method of claim 39, wherein the adjuvantation system enhances the production of antigen-specific antibodies, compared to when the viral antigen is administered alone, optionally wherein the anti-specific antibody is of IgG2c type.

41. The method of any one of claims 1-40, wherein the adjuvantation system enhances the production of antigen-specific antibodies targeting a broader range of epitopes, compared to when the viral antigen is administered alone.

42. The method of any one of claims 1-41, wherein the adjuvantation system enhances the production of a cytokine, compared to when the viral antigen is administered alone.

43. The method of claim 42, wherein the cytokine comprises IFNγ.

44. The method of any one of claims 1-43, wherein the adjuvantation system polarizes the innate immune response toward T follicular helper (Tfh) cell immunity.

45. The method of any one of claims 1-44, wherein the adjuvantation system polarizes the innate immune response toward T helper 1 (Th1) cell immunity.

46. The method of any one of claims 1-45, wherein the adjuvantation system prolongs a protective effect in the subject against the viral antigen, compared to when the viral antigen is administered alone.

47. The method of any one of claims 1-46, wherein the adjuvantation system increases rate of an immune response, compared to when the viral antigen is administered alone.

48. The method of any one of claims 1-47, wherein the viral antigen produces a same level of immune response against the antigen at a lower dose in the presence of the adjuvantation system, compared to when the viral antigen is administered alone.

49. The method of any one of claims 1-48, wherein the likelihood of antibody disease enhancement (ADE) is reduced in the subject, compared to when the viral antigen is administered alone.

50. An adjuvantation system comprising a fungal polysaccharide for use in inducing an immune response against a virus in a subject in need thereof.

51. An adjuvantation system comprising a fungal polysaccharide and alum for use in inducing an immune response against a virus in a subject in need thereof.

52. An immunogenic composition comprising a viral antigen and an adjuvantation system comprising a fungal polysaccharide.

53. The immunogenic composition of claim 52, wherein the fungal polysaccharide is a soluble polysaccharide.

54. The immunogenic composition of claim 53, wherein the fungal polysaccharide is a mannan.

55. The immunogenic composition of claim 54, wherein the fungal polysaccharide is isolated from Candida albicans.

56. The immunogenic composition of any of claims 52-55 wherein the adjuvantation system further comprises alum.

57. The immunogenic composition of claim 56, wherein the fungal polysaccharide is adsorbed into the alum.

58. The immunogenic composition of any one of claims 52-57, wherein the virus is selected from Middle East Respiratory Syndrome coronavirus (MERS-CoV), Severe Acute Respiratory Syndrome (SARS)-associated coronavirus (SARS-CoV)-1, and SARS-CoV-2.

59. The immunogenic composition of any one of claims 52-58, wherein the viral antigen comprises a Beta coronavirus protein or polypeptide.

60. The immunogenic composition of any one of claims 52-58, wherein the viral antigen comprises a nucleic acid encoding a Beta coronavirus protein or a polypeptide.

61. The immunogenic composition of claim 60, wherein the nucleic acid is DNA or RNA.

62. The immunogenic composition of claim 61, wherein the RNA is a messenger RNA (mRNA).

63. The immunogenic composition of any one of claims 59-62, wherein the Beta coronavirus protein or polypeptide comprises a Beta coronavirus spike protein or spike protein receptor binding domain (RBD).

64. The immunogenic composition of claim 63, wherein the Beta coronavirus spike protein is a MERS-CoV spike protein, SARS-CoV-1 spike protein, or SARS-CoV-2 spike protein.

65. The immunogenic composition of claim 64, wherein the Beta coronavirus spike protein RBD is a MERS-CoV spike protein RBD, SARS-CoV-1 spike protein RBD, or SARS-CoV-2 spike protein RBD.

66. The immunogenic composition of any one of claims 52-58, wherein the viral antigen comprises a viral particle of MERS-CoV, SARS-CoV-1, or SARS-CoV-2.

67. The immunogenic composition of any one of claims 52-58, wherein the viral antigen comprises killed or inactivated MERS-CoV, SARS-CoV-1, or SARS-CoV-2.

68. The immunogenic composition of any one of claims 52-58, wherein the viral antigen comprises killed or live attenuated MERS-CoV, SARS-CoV-1, or SARS-CoV-2.

69. The immunogenic composition of any one of claims 52-57, wherein the virus is an influenza A virus or an influenza B virus.

70. The immunogenic composition of any one of claims 52-57 and claim 69, wherein the viral antigen comprises an influenza A virus or influenza B virus protein or polypeptide.

71. The immunogenic composition of any one of claims 52-57 and claim 69, wherein the viral antigen comprises a nucleic acid encoding an influenza A virus or influenza B virus protein or polypeptide.

72. The immunogenic composition of claim 71, wherein the nucleic acid is DNA or RNA.

73. The immunogenic composition of claim 72, wherein the RNA is a mRNA.

74. The immunogenic composition of any one of claims 70-73, wherein the influenza A virus or influenza B virus protein or polypeptide is a hemagglutinin (HA) protein, a neuraminidase (NA) protein, or polypeptide thereof.

75. The immunogenic composition of any one of claims 52-57 and claim 69, wherein the viral antigen comprises a viral particle of influenza A or influenza B.

76. The immunogenic composition of any one of claims 52-57 and claim 69, wherein the viral antigen comprises killed or inactivated influenza A or influenza B.

77. The immunogenic composition of any one of claims 52-57 and claim 69, wherein the viral antigen comprises killed or live attenuated influenza A or influenza B.


SEQUENCE LISTING (19 sequences)

SEQ ID NO: 1 - protein, 1255 aa, Unknown
  SARS-CoV-1
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 222 aa, Unknown
  SARS-CoV-1
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 3 - protein, 76 aa, Unknown
  SARS-CoV-1
MYSFVSEETGTLIVNSVLLFLAFVVFLLVTLAILTALRLCAYCCNIVNVSLVKPTVYVYSRVKNLNSSEGVPDLLV

SEQ ID NO: 4 - protein, 221 aa, Unknown
  SARS-CoV-1
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 5 - protein, 422 aa, Unknown
  SARS-CoV-1
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 6 - protein, 1273 aa, Unknown
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 223 aa, Unknown
  SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 8 - protein, 75 aa, Unknown
  SARS-CoV-2
MYSFVSEETGTLIVNSVLLFLAFVVFLLVTLAILTALRLCAYCCNIVNVSLVKPSFYVYSRVKNLNSSRVPDLLV

SEQ ID NO: 9 - protein, 222 aa, Unknown
  SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 10 - protein, 419 aa, Unknown
  SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 1353 aa, Unknown
  MERS-CoV
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSTVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 12 - protein, 122 aa, Unknown
  MERS-CoV
ECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKC

SEQ ID NO: 13 - protein, 82 aa, Unknown
  MERS-CoV
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 14 - protein, 219 aa, Unknown
  MERS-CoV
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 15 - protein, 413 aa, Unknown
  MERS-CoV
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 16 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQTADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 17 - protein, 469 aa, Influenza A virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 18 - protein, 584 aa, Influenza B virus
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 19 - protein, 466 aa, Influenza B virus
MLPSTVQTLTLLLTSGGVLLSLYVSASLSYLLYSDVLLKFSSTKTTAPTMSLECTNASNAQTVNHSATKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL